(19) United States
(12) Patent Application Publication
(10) Pub. No.: US 2006/0177436 A1
(43) Pub. Date: Aug. 10, 2006

(54) Methods for Treating Autoimmune Disorders

(75) Inventors: Nico Ghilardi, Milbrae, CA (US); Frederic DeSauvage, Foster City, CA (US)

Correspondence Address: MERCHANT & GOULD PC, P.O. BOX 2903, MINNEAPOLIS, MN, 55402-0903, US

(73) Assignee: GENENTECH, INC., South San Francisco, CA (US)

(21) Appl. No.: 11/275,181

(22) Filed: Dec. 16, 2005

Related U.S. Application Data
(60) Provisional application No. 60/636,846, filed on Dec. 16, 2004.
(60) Provisional application No. 60/748,367, filed on Dec. 6, 2005.

(51) Int. Cl. A61K 39/395

(52) U.S. Cl. 424/133.1; 424/143.1

(57) ABSTRACT

The present invention relates to methods for treating autoimmune disorders. In an embodiment, the invention is directed to a method for treating an autoimmune disorder comprising administering a TCCR agonist. In an embodiment, the autoimmune disorder is at least partially mediated by a Th1 response. In an embodiment, the autoimmune disorder is at least partially mediated by CD8⁺ T-cell proliferation.

### BACKGROUND OF THE INVENTION

Autoimmune disorders are the manifestation or consequence of complex, interconnected biological pathways. In normal physiology, these biological pathways are critical for responding to insult or injury, initiating repair from insult or injury, and mounting innate and acquired defenses against foreign organisms. Disease or pathology can occur when these normal physiological pathways cause additional insult or injury, either as related to the intensity of the response, as a consequence of abnormal regulation or excessive stimulation, as a reaction to self, or a combination of these.

Though the genesis of these disorders often involves multi-step pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

The immune system of mammals consists of a number of unique cells that act in concert to defend the host from invading bacteria, viruses, toxins, and other non-host substances. Lymphocytes, both T and B cells, are largely responsible for the specificity of the immune system. T cells take their designation from being developed in the thymus, while B cells develop in the bone marrow.

T lymphocytes (T cells) are an important component of a mammalian immune response. T cells recognize antigens that are associated with a self-molecule encoded by genes within the major histocompatibility complex (MHC). The antigen may be displayed together with MHC molecules on the surface of antigen presenting cells, virus infected cells, cancer cells, grafts, and the like. The T cell system eliminates these altered cells that pose a health threat to the host mammal. T cells include helper T cells (CD4⁺) and cytotoxic T-lymphocytes (CD8⁺). Helper T cells (TH) proliferate extensively following recognition of an antigen-MHC complex on an antigen presenting cell. Helper T cells also secrete a variety of cytokines, such as lymphokines, that play a central role in the activation of B cells, cytotoxic T-lymphocytes, and a variety of other cells that participate in the immune response. Cytotoxic T-lymphocytes are able to cause the destruction of other cells.

A central event in both humoral and cell mediated immune responses is the activation and clonal expansion of helper T cells. Helper T cell activation is initiated by the interaction of the T cell receptor (TCR)-CD3 complex with an antigen-MHC on the surface of an antigen presenting cell. This interaction mediates a cascade of biochemical events that induce the resting helper T cell to enter a cell cycle (the G0 to G1 transition) and results in the expression of a high affinity receptor for IL-2. The activated T cell progresses through the cycle proliferating and differentiating into memory cells or effector cells.

The T-helper cell subsets (Th1 and Th2) define 2 pathways of immunity: cell-mediated immunity and humoral immunity. Release profiles of cytokines for Th1 and Th2 subtypes influence selection of effector mechanisms and cytotoxic cells (Mosmann et al., 1989, Adv. Immunol., 46:111-147; Mosmann et al., 1996, Immunol. Today, 17:138-146). Th1 cells, a functional subset of CD4⁺ cells, are characterized by their ability to boost cell-mediated immunity and produce cytokines including Il-2, interferon-gamma, and lymphotoxin beta (Mosmann et al., 1989, 1996, supra). Il-2 and interferon-gamma secreted by Th1 cells activate macrophages and cytotoxic cells. Th2 cells are also CD4+ cells, but are distinct from Th1 cells. Th2 cells are characterized by their ability to boost humoral immunity, such as antibody production. Th2 cells produce cytokines, including Il-4, Il-5, and Il-10 (Mosmann et al., 1989, 1996, supra). Il-4, Il-5, and Il-10 secreted by Th2 cells increase production of eosinophils and mast cells, as well as enhance production of antibodies, including IgE, and decrease the function of cytotoxic cells (Powrie et al., 1993, Immunol Today, 14:270).

Th1 and Th2 cytokine release modulate the mutually inhibitory Th1 and Th2 responses. For example, IL-4 inhibits the expression of interferon-gamma from Th1 cells whereas interferon-gamma inhibits the expression of IL-4 from Th2 cells (Mosmann et al., 1989, supra).

Members of the four helical bundle cytokine family (Bazan, 1990, PNAS, 87:6934) modulate expansion and terminal differentiation of T helper cells from a common precursor into distinct populations of Th1 and Th2 effector cells (O'Garra, A., 1998, Immunity, 8:275-83). For example, IL-4 influences development of Th2 cells, while IL-12 is involved in differentiation of Th1 cells (Hsieh et al., 1993, Science, 260:547-9; Seder et al., 1993, PNAS, 90:10188-92).

TCCR (T-Cell Cytokine Receptor) is of the WS(G)XWS class of cytokine receptors with homology to the IL-12 β-2 receptor, G-CSFR, and IL-6 receptor. These receptors transduce a signal that can control growth and differentiation of cells, especially cells involved in blood cell growth and differentiation. TCCR has been suggested to be involved in the T-helper cell response. Specifically, it has been posited that TCCR and its ligand IL-27 promote Th1 responses (Chen et al., 2000, Nature, 407:916-920; Yoshida et al., 2001, Immunity, 15:569-578; Pflanz et al., 2002, Immunity, 16:779-790).

Overproduction of cytokines produced by either or both of Th1 and Th2 cells impacts a host of medical disorders. For example, overproduction of Th1 cytokines contributes to pathogenesis of various autoimmune disorders, such as multiple sclerosis and rheumatoid arthritis. Overproduction of Th2 cytokines contributes to pathogenesis of allergic disorders.

CD8⁺ cytotoxic T-lymphocytes (CTLs) are involved in pathogenic destruction of tissue in some autoimmune diseases. For example, CTLs are implicated in destruction of pancreatic β cells during the course of autoimmune type I diabetes (Kagi et al., 1997, J. Exp. Med., 186:989-997). CTLs are also implicated in experimental autoimmune encephalomyelitis (Huseby et al., 2001, J. Exp. Med., 194(5):669-676). CTLs mediate tissue damage associated with graft-versus host disease (GVHD) (Graubert et al., 1997, J. Clin. Invest., 100:904-911).

Multiple Sclerosis (MS) is a disorder of the central nervous system that affects the brain and spinal cord. Common signs and symptoms of MS include paresthesias in one or more extremities, in the trunk, or on one side of the face; weakness or clumsiness of a leg or hand; or visual disturbances (such as partial blindness and pain in one eye), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances (Berkow et al. (ed.), 1999, Merck Manual of Diagnosis and Therapy: 17th Ed). Current treatments for MS include corticosteroids, beta interferons (Betaferon, Avonex, Rebif), glatiramer acetate (Copaxone), methotrexate, azathioprine, cyclophosphamide, cladribine, baclofen, tizanidine, amitriptyline, carbamazepine (Berkow et al. (ed.), 1999, supra).

Rheumatoid arthritis (RA) is a chronic autoimmune disorder characterized by synovitis of joints that typically affects small and large joints, leading to their progressive destruction (Berkow et al. (ed.), 1999, supra). Symptoms of RA can include stiffness, tenderness, synovial thickening, flexion contractures, visceral nodules, vasculitis causing leg ulcers or mononeuritis multiplex, pleural or pericardial effusions, and fever (Berkow et al. (ed.), 1999, supra).

Current treatments for RA include non-steroidal anti-inflammatory drugs (including salicylates), gold compounds, methotrexate, hydroxychloroquine, sulfasalazine, penicillamine, corticosteroids, and cytotoxic or immunosuppressive drugs. (Berkow et al. (ed.), 1999, Merck Manual of Diagnosis and Therapy: 17th Ed.).

None of the existing therapies for autoimmune disorders have proven to be satisfactory because of limited efficacy and/or significant toxicity. Thus, new methods for treating autoimmune disorders such as MS and RA are needed.

### SUMMARY OF THE INVENTION

Naieve, undifferentiated T cells (Th-0) respond to different signals that induce differntiation of Th-0 cells into mature T-helper cells. It has now been discovered that activation of cellular receptor TCCR, for example by administering an agonist of TCCR such as IL-27, is effective to reduce T-lymphocyte proliferation. Reduction in T-lymphocyte proliferation was correlated with increased expression of IL-10 and SOCS-3. Animals expressing TCCR have been found to be less susceptible to autoimmune disease.

Further studies in the EAE disease model indicated that IL-27 receptor (TCCR)-deficient mice are hypersensitive to autoimmune disease. Study of the role of IL-27 in Th-cell differentiation and in immune disorders led to the surprising discovery that IL-27 is immunosupressive, acting at multiple levels in Th development. IL-27 supresses production of Th-_{IL17} cells, inhbits production of IL-6, and inhibits productioin of Th_{IL17} cytokines, including IL-6. IL-27 induces production of IL-10, and of IL-4, a further inhibitor of Th-_{IL17} cells, and stimulates production of IL12 receptor and differentiation of Th-1 cells. The data disclosed herein indicate that IL-27 has an important immunosupressive function, including important inhibitory activity across Th-1, Th-2 and Th-17 cells.

The invention provides methods for treating autoimmune disorders including multiple sclerosis (MS) and rheumatoid arthritis (RA), by administering an agonist of the IL27R (TCCR) such as IL-27. Useful agonists of TCCR include variants and fragments of IL27R, IL27R ligands such as IL-27 and variants and fragments thereof, as well as agonist antibodies that bind IL27R or a IL27R ligand and stimulate, induce, or enhance a IL27-mediated response. The invention also provides methods of inhibiting proliferation of T-lymphocytes and/or cytotoxic T-lymphocytes, including Th-_{IL17} cells, the method comprising administering a agonist that stimulates, enduces, or enhances an IL27/IL27R response.

### BRIEF DESCRIPTION OF THE DRAWINGS

FIG. 1 is a diagrammatic representation of the architecture of the TCCR/IL-27 receptor complex.

FIG. 2 is a graph showing the proliferation of Ba/F3 cells expressing human TCCR in response to monoclonal antibodies: 2685-IgG2a, 2686-IgG1, 2688-IgG1, control isotype IgG2a, and control isotype IgG1.

FIG. 3 is a graph showing proliferation of Ba/F3 cells expressing human TCCR in response to murine IL-3 (positive control) and antibody 2686.

FIG. 4 is a graph showing proliferation of Ba/F3 cells expressing human TCCR, murine TCCR, and control cells expressing neither, in response to antibody 2686.

FIG. 5 is a graph showing proliferation of splenocytes expressing TCCR in response to anti-CD3 stimulation in comparison to proliferation of splenocytes not expressing TCCR.

FIG. 6 is a graph showing proliferation of CD4⁺ T cells expressing TCCR in response to anti-CD3 stimulation in comparison to proliferation of CD4+ T cells not expressing TCCR.

FIG. 7 is a graph showing proliferation of CD8⁺ T cells expressing TCCR in response to anti-CD3 stimulation in comparison to proliferation of CD8⁺ T cells not expressing TCCR.

FIG. 8 is a graph showing the clinical progression of MOG induced EAE in knockout (TCCR −/−) and wild-type (TCCR +/+) mice.

FIG. 9 is a graph showing the clinical progression of MBP induced EAE in knockout (TCCR −/−) and wild-type (TCCR +/+) mice.

FIG. 10 is a graph showing average histological inflammation scores for brain and spinal cord sections for knockout (TCCR −/−) and wild-type (TCCR +/+) mice in an EAE model.

FIG. 11 is a graph showing is a graph showing average histological demyelination scores for brain and spinal cord sections for knockout (TCCR −/−) and wild-type (TCCR +/+) mice in an EAE model.

FIG. 12 is a graph showing maximum histological inflammation scores for brain and spinal cord sections for knockout (TCCR −/−) and wild-type (TCCR +/+) mice in an EAE model.

FIG. 13 is a graph showing maximum histological demyelination scores for brain and spinal cord sections for knockout (TCCR −/−) and wild-type (TCCR +/+) mice in an EAE model.

FIG. 14 is a graph showing proliferation of CD4⁺ T cells expressing TCCR in response to anti-CD3 stimulation in comparison to proliferation of CD4⁺ T cells not expressing TCCR in a CFSE labeling assay.

FIG. 15 is a graph showing proliferation of CD8⁺ T cells expressing TCCR in response to anti-CD3 stimulation in comparison to proliferation of CD8⁺ T cells not expressing TCCR in a CFSE labeling assay.

FIGS. 16A-C are graphs showing the induction of IL-2 in response to treatment with IL-27 at various time points under neutral (16A), Th1 biasing (16B), and Th2 biasing (16C) conditions. Data are represented as fold IL-27 dependent induction.

FIGS. 17A-C are graphs showing the induction of IL-10 in response to treatment with IL-27 at various time points under neutral (17A), Th1 biasing (17B), and Th2 biasing (17C) conditions. Data are represented as fold IL-27 dependent induction.

FIGS. 18A-C are graphs showing the induction of SOCS-3 in response to treatment with IL-27 at various time points under neutral (18A), Th1 biasing (18B), and Th2 biasing (18C) conditions. Data are represented as fold IL-27 dependent induction.

FIG. 19 is a graph showing proliferation of CD4⁺ cells in response to IL-27 treatment under neutral, Th1 biasing, and Th2 biasing conditions.

FIGS. 20A-B are graphs showing proliferation of splenocytes in response to IL-27 and/or IL-6 treatment in the absence (20A) or presence (20B) of anti-IL-2 antibodies.

FIG. 21 is a diagram of IL-27 and its receptor IL-27R (TCCR).

FIG. 22 is a diagram showing the relationship of IL-27 to IL-6 cluster of cytokines, within the IL-12 cytokine group.

FIG. 23 is a diagram showing differentiation of helper T-cells.

FIG. 24 is a graph showing hypersensitivity to EAE in IL-27R deficient mice.

FIG. 25 shows histological analysis of EAE phenotype in wild type and IL-27R knockout mice.

FIG. 26 is a schematic digram of a protocol testing the relationship of IL-27 in T-cells.

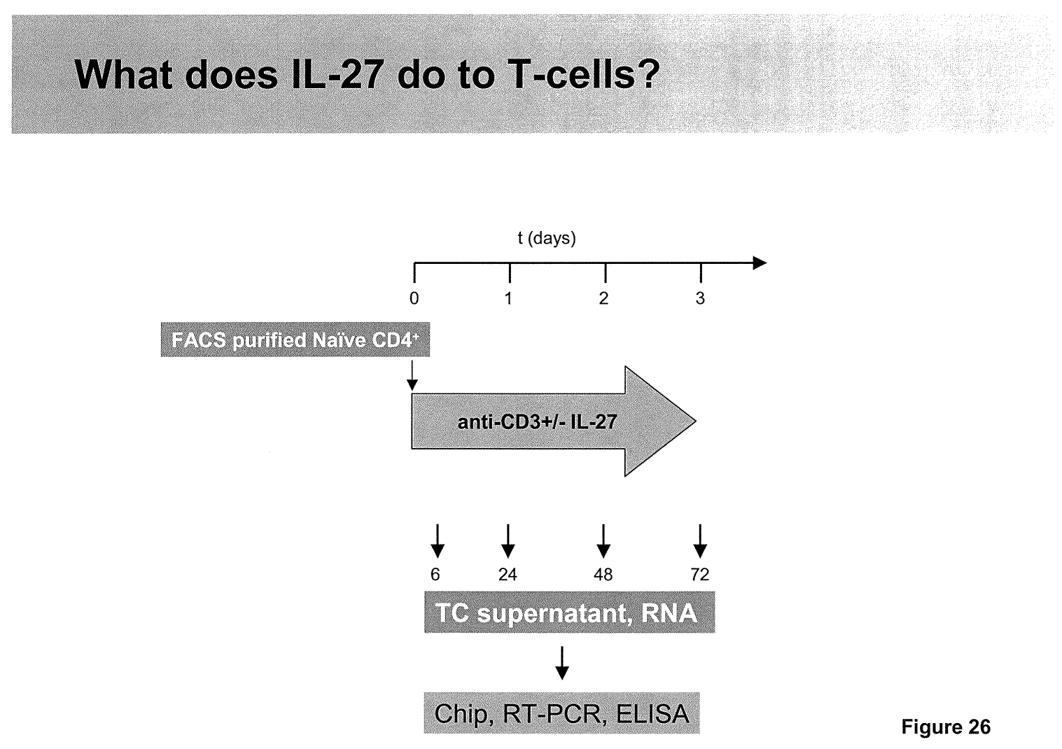

FIG. 27 shows the results of testing of the effects of IL-27 on T-cell development. Cytokine reduction in response to IL-27 is compared with wild type conrol for the following cytokines: IFN-gamma, IL-2, TFN, IL-4, IL-5, IL-6, IL-10, GM-CSF, and IL-17.

FIG. 28 graphically shows the results of IL-2 and GM-CSF production in response to IL-10, IL-27, and a combination of IL-10 and IL-27.

FIG. 29 graphically shows strong induction of IL-10 in response to IL-27, the severity of EAE in IL-10 knockout mice.

FIG. 30 is a diagram delineating the role of TH-17 in EAE.

FIG. 31 graphically shows the requirements of IL-23 and TH-IL-17 cells for EAE disease.

FIG. 32 graphically demonsrtates suppression of TH-IL-17 cytokines by IL-27.

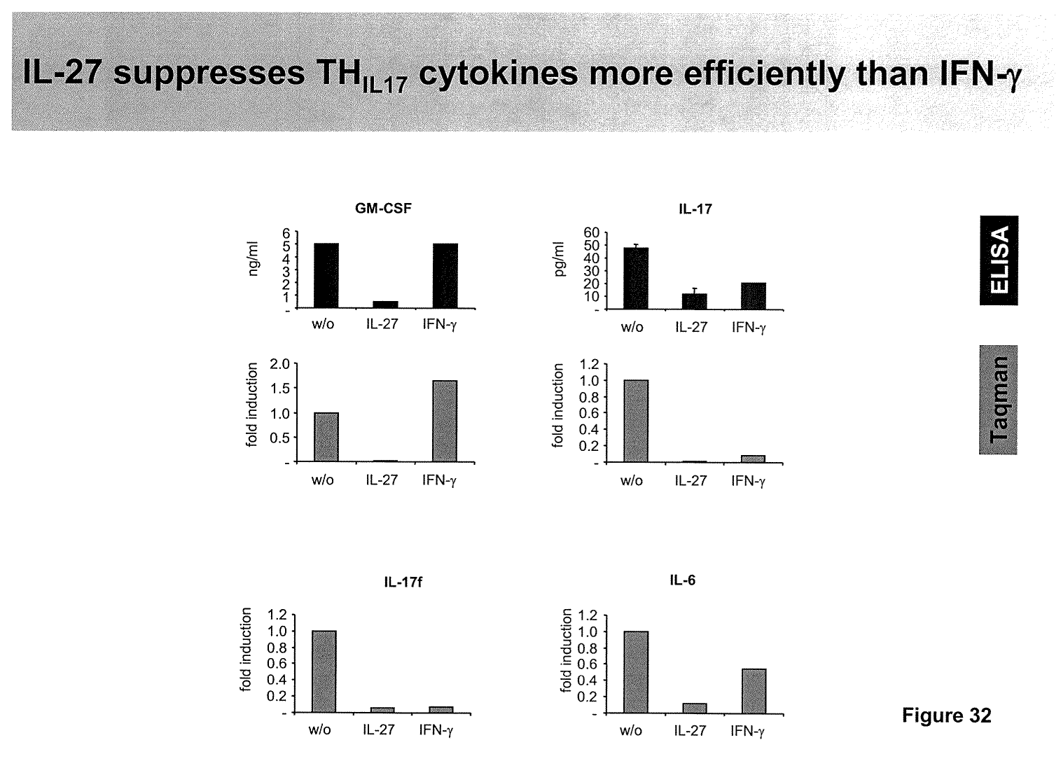

FIG. 33 graphically shows the suppression of IL-17 by IL-27.

FIG. 34 graphically shows suppression of IL-17 mediated by IL-27 is IFNg independent.

FIG. 35 graphically shows suppression of IL-17 by IL-27 is mediated by STAT-1.

FIG. 36 graphically shows secretion of TH-IL-17 cytokines from IL-17-R knockout mice from restimulated lyphocytes of IL-17-R knockout mice.

FIG. 37 shows IL-17 production in response to disease inducing MOG or KLH in IL-27-R deficient mice.

FIG. 38 graphically shows IL-17 expression by CD4T cells infiltrating the CNS.

FIG. 39 is a diagram demonstrating the relationship of various cytokines to T helper differentiation.

FIG. 40 is a graph demonstating EAE resistance in IL-6 knockout mice.

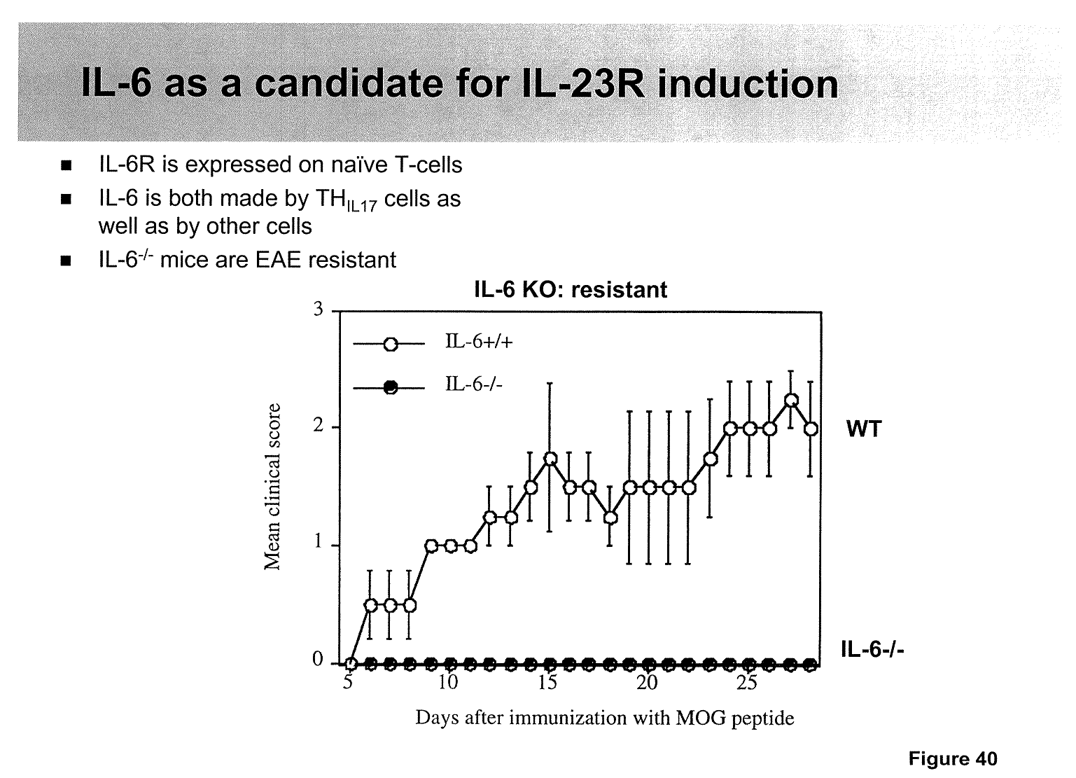

FIG. 41 graphically demonstates induction of TH-IL-17 cytokines and response by IL-6.

FIG. 42 is a graph demonstrating the antagonism of IL-6 proliferation effects by IL-27.

FIG. 43 is a diagram demonstrating the role of IL-27 and IL-6 and TH-IL-17 development.

FIG. 44 is a diagram demonstrating the multiple levels of action of IL-27.

FIG. 45 graphically illustrates the role of IL-27 in inducing changes in cytokine expression.

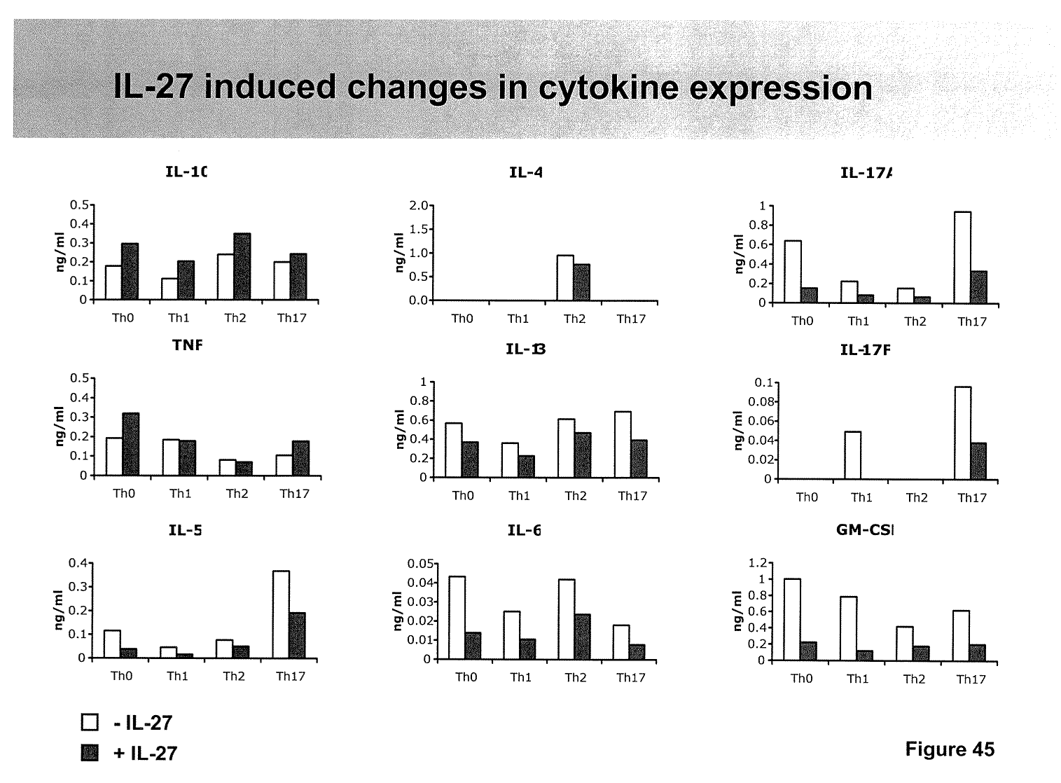

### BRIEF DESCRIPTION OF THE SEQUENCES

TABLE 24

| SEQ ID Number: | Sequence Of: | Page Number: |
|---|---|---|
| 1 | Human TCCR (AA) | Table 1, Pages 22-23 |
| 2 | Murine TCCR (AA) | Table 1, Pages 22-23 |
| 3 | Human p28 (AA) | Table 2, Pages 24-25 |
| 4 | Murine p28 (AA) | Table 2, Pages 24-25 |
| 5 | Human EBI3 (AA) | Table 3, Page 25 |
| 6 | Human gp130 (AA) | Table 4, Pages 26-27 |
| 7 | MOG 35-55 (AA) | Example 2, Page 42 |
| 8 | Ac 1-11 (AA) | Example 2, Page 43 |
| 9 | mSOCS1 forward (NT) | Table 17, Page 56 |
| 10 | mSOCS1 reverse (NT) | Table 17, Page 56 |
| 11 | mSOCS1 probe (NT) | Table 17, Page 56 |
| 12 | mSOCS3 forward (NT) | Table 17, Page 56 |
| 13 | mSOCS3 reverse (NT) | Table 17, Page 56 |
| 14 | mSOCS3 probe (NT) | Table 17, Page 56 |
| 15 | mPIAS1 forward (NT) | Table 17, Page 56 |
| 16 | mPIAS1 reverse (NT) | Table 17, Page 56 |
| 17 | mPIAS1 probe (NT) | Table 17, Page 56 |
| 18 | mPIAS3 forward (NT) | Table 17, Page 56 |
| 19 | mPIAS3 reverse (NT) | Table 17, Page 56 |
| 20 | mPIAS3 probe (NT) | Table 17, Page 56 |

### DETAILED DESCRIPTION

Over-proliferation of T-lymphocytes or over-production of cytokines produced by Th1 or Th2 cells leads to a host of medical disorders. For example, over-production of cytokines associated with a Th1 response or over-proliferation of CD8⁺ cytotoxic T-lymphocytes can lead to autoimmune disorders including allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), insulin-dependent diabetes mellitus, multiple sclerosis, pernicious anemia, psoriasis, rheumatoid arthritis, sarcoidosis, scleroderma, and systemic lupus erythematosus.

Studies detailed in the Examples below demonstrated greater proliferation of T cells lacking TCCR than T cells expressing TCCR in response to non-specific T cell stimulation (see Example 1). Previously, it was suggested that TCCR and its ligand IL-27 promote Th1 responses (Chen et al., 2000, Nature, 407:916-920; Yoshida et al., 2001, Immunity, 15:569-578; Pflanz et al., 2002, Immunity, 16:779-790). However, it was surprisingly discovered that mice expressing TCCR were less susceptible to autoimmune disease characterized in part by a Th1 response, such as experimental allergic encephalomyelitis (EAE), an animal model for multiple sclerosis, than were mice lacking TCCR (see Example 2).

As shown in the Examples below, proliferation of T lymphocytes is inhibited by administration of a TCCR agonist to the cells. Also shown, reduced clinical progression and less severe symptoms of autoimmune inflammatory disease are present in animals expressing TCCR (TCCR+/+) than in TCCR−/− animals.

These data show that agonists of TCCR can be used to reduce T-cell proliferation. In particular, the data show that agonists of TCCR are useful to treat autoimmune mediated disorders such as multiple sclerosis (MS) and rheumatoid arthritis (RA).

### DEFINITIONS

The term “autoimmune” refers to the process by which immune system components such as antibodies or lymphocytes attack or harm molecules, cells, or tissues of the organism producing them.

The term “autoimmune disorders” refers to diseases where damage, such as tissue damage, or pathogenesis is, at least partially, a result of an autoimmune process. By way of example, the term “autoimmune disease” includes those diseases that are mediated at least partially by a Th1 response or CD8⁺ cytotoxic T-lymphocytes. Autoimmune diseases include allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), insulin-dependent diabetes mellitus, multiple sclerosis, pernicious anemia, psoriasis, rheumatoid arthritis, sarcoidosis, scleroderma, and systemic lupus erythematosus.

The term “Th1 response” refers to differentiation of T helper cells from precursors into distinct populations of Th1 effector cells, and includes secretion of cytokines from Th1 cells, such as IFN-gamma, IL-2, and TNF-beta. The term “Th 1 biasing conditions” refers to conditions that favor the differentiation of T helper cells from precursors into distinct populations of Th1 effector cells.

The term “Th1 cytokines” refers to those cytokines expressed in a Th1 response, including IFN-gamma, IL-2, and TNF-beta. (Powrie et al., 1993, Immunol. Today, 14:270.) The term “Th1 mediated disorder” refers to a disorder mediated predominantly or partially by overproduction of Th1 cytokines. The term “Th1 mediated disorder” includes those disorders that may result from an overproduction or bias in the differentiation of T-cells into the Th1 subtype. Such disorders include autoimmune disorders, for example, RA and MS.

The term “Th2 response” refers to differentiation of T helper cells from precursors into distinct populations of Th2 effector cells, and includes secretion of cytokines from Th2 cells, such as IL-4, IL-5, IL-10, and IL-13. (Powrie et al., 1993, Immunol. Today, 14:270.) The term “Th 2 biasing conditions” refers to conditions that favor the differentiation of T helper cells from precursors into distinct populations of Th2 effector cells.

The terms “TCCR peptide”, “TCCR protein” and “TCCR” when used herein, encompass native sequence TCCR and TCCR peptide variants. TCCR peptide may be isolated from a variety of sources, such as human tissue or another source, or prepared by recombinant and/or synthetic methods. A “native sequence TCCR” is a peptide having the same amino acid sequence as a TCCR peptide derived from nature. Such native sequence TCCR can be isolated from nature or can be produced by recombinant and/or synthetic means. The term “native sequence TCCR” specifically encompasses naturally-occurring truncated and secreted forms (such as an extracellular domain sequence), naturally-occurring truncated forms (such as alternatively spliced forms), and naturally-occurring allelic variants of TCCR. In one embodiment, native sequence human TCCR is a mature or full-length native sequence TCCR comprising amino acids 1 to 636 of SEQ ID NO: 1. Similarly, native sequence murine TCCR is a mature or full-length native sequence TCCR comprising amino acids 1 to 623 of SEQ ID NO:2. While SEQ ID NO: 1 and SEQ ID NO:2 are shown to begin with the methionine residue designated herein as amino acid position 1, it is conceivable and possible that another methionine residue located either upstream or downstream from amino acid position 1 of SEQ ID NO:1 or SEQ ID NO:2 may be employed as the starting amino acid residue for the TCCR peptide.

“TCCR peptide extracellular domain” or “TCCR ECD” refers to a form of the TCCR peptide that is essentially free of transmembrane and cytoplasmic domains. Ordinarily, a TCCR peptide ECD will have less than about 1% of such transmembrane and/or cytoplamic domains and preferably, will have less than about 0.5% of such domains. It will be understood that any transmembrane domain(s) identified for the TCCR peptides of the present invention are identified pursuant to criteria routinely employed for identifying that type of hydrophobic domain. The exact boundaries of a transmembrane domain may vary but most likely be no more than about 5 amino acids at either end of the domain as initially identified. As such, in one embodiment, the extracellular domain of a human TCCR peptide comprises amino acids 1 or about 33 to X₁, where X₁ is any amino acid residue from residue 512 to residue 522 of SEQ ID NO:1. Similarly, the extracellular domain of the murine TCCR peptide comprises amino acids 1 or about 25 to X₂, where X₂ is any amino acid residues from residue 509 to residue 519 of SEQ ID NO:2.

The term “TCCR variant peptide” means a peptide having at least one biological activity of TCCR peptide and having at least about 80% amino acid sequence identity with the amino acid sequence of:
(a1) residue 1 or about 33 to 636 of the human TCCR peptide of SEQ ID NO:1;
(a2) residue 1 or about 25 to 623 of the murine TCCR peptide of SEQ ID NO:2;
(b1) X3 to 636 of the human TCCR peptide of SEQ ID NO:1, where X3 is any amino acid residue 27 to 37 of SEQ ID NO:1;
(b2) X4 to 623 of the murine TCCR peptide of SEQ ID NO:2, where X4 is any amino acid residue from 20 to 30 of SEQ ID NO:2;
(c1) 1 or about 33 to X1, where X1 is any amino acid residue from residue 512 to residue 522 of SEQ ID NO:1;
(c2) 1 or about 25 to X2, where X2 is any amino acid residue from residue 509 to 519 of SEQ ID NO:2;
(d1) X5 to 636, where X5 is any amino acid from residue 533 to 543 of SEQ ID NO:1;
(d2) X6 to 623, where X6 is any amino acid from residue 527 to 537 of SEQ ID NO:2; or
(e) another specifically derived fragment of the amino acid sequences of SEQ ID NO:1 and SEQ ID NO:2.

Such TCCR variant peptides include, for instance, TCCR peptides where one or more amino acid residues are added, or deleted, at the N— and/or C-terminus, as well as within one or more internal domains, of the sequence of SEQ ID NO:1 and SEQ ID NO:2. Ordinarily, a TCCR variant peptide will have at least about 80% amino acid sequence identity and can be at least about 81%, 82%, 83%, 84%, 85%, 86%, 87%, 88%, 89%, 90%, 91%, 92%, 93%, 94%, 95%, 96%, 97%, 98%, or 99% amino acid sequence identity with:
(a1) residue 1 or about 33 to 636 of the human TCCR peptide of SEQ ID NO:1;
(a2) residue 1 or about 25 to 623 of the murine TCCR peptide of SEQ ID NO:2;
(b1) X3 to 636 of the human TCCR peptide of SEQ ID NO:1, where X3 is any amino acid residue 27 to 37 of SEQ ID NO:1;
(b2) X4 to 623 of the murine TCCR peptide of SEQ ID NO:2, where X4 is any amino acid residue from 20 to 30 of SEQ ID NO:2;
(c1) 1 or about 33 to X1 wherein X1 is any amino acid residue from residue 512 to residue 522 and of SEQ ID NO:1;
(c2) 1 or about 25 to X2, where X2 is any amino acid residue from residue 509 to 519 of SEQ ID NO:2;
(d1) X5 to 636, where X5 is any amino acid from residue 533 to 543 of SEQ ID NO:1;
(d2) X6 to 623, where X6 is any amino acid from residue 527 to 537 of SEQ ID NO:2; or
(e) another specifically derived fragment of the amino acid sequences of SEQ ID NO:1 and SEQ ID NO:2.

The term “IL-27”, when used herein, encompasses native sequence IL-27 heterodimer, native sequence IL-27 components EBI3 and p28, IL-27 heterodimer variants (further defined herein), and variants of EBI3 and p28. The IL-27 heterodimer and components thereof may be isolated from a variety of sources, such as from human tissue types or from another source, or prepared by recombinant and/or synthetic methods. A “native sequence IL-27” comprises a heterodimer having the same amino acid sequence as a IL-27 heterodimer derived from nature. Such native sequence IL-27 heterodimers can be isolated from nature or can be produced by recombinant and/or synthetic means. The term “native sequence IL-27” specifically encompasses naturally-occurring truncated and secreted forms (such as an extracellular domain sequence), naturally-occurring truncated forms (such as alternatively spliced forms), and naturally-occurring allelic variants of the IL-27 heterodimer.

The term “IL-27 variants” refers to those peptides having homology to native sequence IL-27, including native sequence IL-27 components EBI3 and p28, that can activate TCCR. IL-27 variants may include those that are formed from EB13 variants and p28 variants. IL-27 variants may also include those that can engage both TCCR and gp130. IL-27 variants may include those that can form a TCCR homodimer. IL-27 variants include PEGylated IL-27.

The term “p28”, when used herein, encompasses native sequence p28 and p28 peptide variants. p28 may be isolated from a variety of sources, such as from human tissue types or from another source, or prepared by recombinant and/or synthetic methods. A “native sequence p28” comprises a peptide having the same amino acid sequence as a p28 peptide derived from nature. Such native sequence p28 can be isolated from nature or can be produced by recombinant and/or synthetic means. The term “native sequence p28” specifically encompasses naturally-occurring truncated or secreted forms (such as an extracellular domain sequence), naturally-occurring truncated forms (such as alternatively spliced forms) and naturally-occurring allelic variants of the p28.

The term “p28 peptide variants” encompasses peptides having at least 73%, 75%, 80%, 90%, 95%, or 99% sequence identity with native sequence human p28 (SEQ ID NO: 3) or murine p28 (SEQ ID NO: 4). p28 peptide variants include portions of p28 capable of binding TCCR and gp130. p28 peptide variants include portions of p28 capable of activating TCCR. p28 peptide variants include peptides containing residues from the first and third alpha helices of p28, believed to bind TCCR in the region of the cytokine receptor homology domain found on TCCR, and residues at the end of the first helix and the beginning of the fourth helix, believed to bind the IG domain found on gp130.

The term “EBI3” when used herein encompasses native sequence EBI3 and EBI3 peptide variants. The EBI3 peptide may be isolated from a variety of sources, such as from human tissue types or from another source, or prepared by recombinant and/or synthetic methods. A “native sequence EBI3” comprises a peptide having the same amino acid sequence as a EBI3 peptide derived from nature. Such native sequence EBI3 can be isolated from nature or can be produced by recombinant and/or synthetic means. The term “native sequence EBI3” specifically encompasses naturally-occurring truncated and secreted forms (such as an extracellular domain sequence), naturally-occurring truncated forms (such as alternatively spliced forms), and naturally-occurring allelic variants of the EBT3.

The term “fusion protein” refers to, by way of example, an expression product resulting from the fusion of two genes that code for two different proteins. The term also includes an expression product resulting from the fusion of portions of two genes coding for portions of two different proteins. The term includes those proteins resulting from a fusion that takes place post-translationally. As used herein, the term would include IL-27, its components (EBT3 and p28), or portions thereof, fused to a heterologous peptide. The term would also include TCCR or portions thereof, fused to a heterologous peptide. The term would also include EB13 fused to p28 to form a functional one chain cytokine. (Pflanz et al., 2002, Immunity, 16:779-790.) The term includes IL-27 conjugated to a human Fc tag.

As used herein, “heterologous peptide” with respect to a given peptide refers to peptides with different sequences, regardless of origin. For example, with respect to native sequence TCCR, a heterologous peptide refers to a peptide having a sequence other than that of native sequence TCCR. With respect to native sequence IL-27, a heterologous peptide refers to a peptide having a sequence other than that of native sequence IL-27.

The term “agonist” includes any molecule that enhances or stimulates a biological activity of a native sequence peptide Suitable agonist molecules specifically include agonist peptides, agonist antibodies or antibody fragments, fragments or amino acid sequence variants of native peptides of the invention, and the like. Methods for identifying agonists of TCCR include, for example, contacting a TCCR peptide or a TCCR peptide-expressing cell with a candidate agonist molecule and measuring a detectable change in one or more TCCR biological activities.

“TCCR biological activity” as used herein refers to a TCCR mediated response, such as dampening or suppressing T-cell proliferation. TCCR biological activity includes dampening or suppressing a Th1 response or a Th1 mediated disorder. TCCR biological activity includes increasing expression of IL-10 and SOCS-3. TCCR biological activity also includes signaling associated with activation of TCCR, for example phosphorylation of signal transduction and transcription factors such as Stat1, Stat3, Stat4, and Stat5 (Lucas et al., 2003, PNAS, 100(25):15047-52).

The terms “antibody” and “immunoglobulin” are used in the broadest sense and specifically include polyclonal antibodies, monoclonal antibodies (including agonist and antagonist antibodies), multivalent antibodies (such as bivalent antibodies), multispecific antibodies (such as bispecific antibodies that exhibit a desired biological activity), antibody compositions with polyepitopic specificity, affinity matured antibodies, humanized antibodies, human antibodies, chimeric antibodies, as well as antigen binding fragments (such as Fab, F(ab′)₂, scFv, and Fv), that exhibit a desired biological activity. A naturally occurring antibody comprises four peptide chains, two identical heavy (H) chains and two identical light (L) chains inter-connected by disulfide bonds. Each heavy chain comprises a heavy chain variable region domain (V_{H}) and a heavy chain constant region. The heavy chain constant region comprises three domains, CH1, CH2 and CH3. Each light chain comprises a light chain variable region domain (V_{L}) and a light chain constant region domain. The light chain constant region comprises one domain, C_{L}. The V_{H} and V_{L} domains can be further subdivided into complementarity determining regions (CDRs) as defined by sequence (see Kabat et al., 1991, Sequences of Proteins of Immunological Interest, 5th Ed. Public Health Service, National Institutes of Health, Bethesda, Md.) or hypervariable loops (HVLs) as defined by three-dimensional structure (Chothia et al., 1987, J. Mol. Biol., 196:901-917), interspersed with regions that are more conserved, termed framework regions (FR). Each V_{H} and V_{L} is typically composed of three CDRs (or HVLs) and four FRs, arranged from amino-terminus to carboxy-terminus in the following order: FR1, CDR1 (HVL1), FR2, CDR2 (HVL2), FR3, CDR3 (HVL3), FR4.

Antibodies (immunoglobulins) are assigned to different classes, depending on the amino acid sequences of the constant domains of their heavy chains. There are five major classes of immunoglobulins: IgA, IgD, IgE, IgG, and IgM, and several of these are further divided into subclasses (isotypes), such as IgG1, IgG2, IgA1, IgA2, and the like. The heavy chain constant domains that correspond to the different classes of immunoglobulins are called alpha, delta, epsilon, gamma, and mu, respectively. The subunit structures and three-dimensional configurations of different classes of immunoglobulins are well known and described generally in, for example, Abbas et al., 2000, Cellular and Mol. Immunology, 4th ed. An antibody may be part of a larger fusion molecule, formed by covalent or non-covalent association of the antibody with one or more other proteins or peptides.

The term “full-length antibody” refers to an antibody in its substantially intact form, including at least 2 heavy and 2 light chains, and not antibody fragments as defined below. The term particularly refers to an antibody with heavy chains that contain Fc regions. A full-length antibody can be a native sequence antibody or a recombinant antibody. A full-length antibody can be human, humanized, and/or affinity matured.

The term “monoclonal antibody” as used herein refers to an antibody obtained from a population of substantially homogeneous antibodies, i.e., the individual antibodies comprising the population are essentially identical except for variants that may arise during production of the antibody.

Monoclonal antibodies described herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., 1984, PNAS, 81:6851-6855.

“Humanized” forms of non-human (e.g., murine) antibodies are chimeric immunoglobulins, immunoglobulin chains, or fragments thereof (such as Fv, Fab, Fab′, F(ab′)₂, or other antigen-binding subsequences of antibodies) that contain minimal sequence derived from non-human immunoglobulins. For the most part, humanized antibodies are human immunoglobulins (recipient antibody) in which residues from one or more complementarity determining regions (CDR) or hypervariable loops (HVL) of the recipient are replaced by residues from one or more CDRs or HVLs of a non-human species (donor antibody) such as mouse, rat, or rabbit having the desired antigen specificity, affinity, and capacity. In some instances, specific Fv framework region (FR) residues of the human immunoglobulin are replaced by corresponding non-human residues. Furthermore, humanized antibodies may comprise residues that are not found in the recipient antibody nor in the imported CDR (or HVL) or in the framework sequences. These modifications are made to further refine and maximize antibody performance. In general, the humanized antibody will comprise substantially all of at least one, and typically two, variable domains, in which all or substantially all of the CDRs or HVLs correspond to those of a non-human immunoglobulin and all or substantially all of the FRs are those of a human immunoglobulin sequence.

A choice of human variable domains, both light and heavy, can be used in making humanized antibodies. According to the “bestfit” method, the sequence of the variable domain of a rodent antibody, for example, is screened against the entire library of known human variable-domain sequences. The human sequence that is closest to that of the rodent is used as the human framework region (FR) for the humanized antibody (Sims et al., 1993, J. Immunol., 151 :2296. Alternatively, the recipient framework region can be derived from a human antibody consensus sequence for a particular subgroup of light or heavy chains. The same framework may be used or modified and used to produce several different humanized antibodies (Carter et al., 1992, Proc. Natl. Acad. Sci. USA, 89:4285; Prestaetal., 1993, J. Immunol., 151:2623). The humanized antibody optionally comprises at least a portion of an immunoglobulin constant region (Fc), typically that of a human immunoglobulin. For further details, see for example Jones et al., 1986, Nature, 321:522-525; Reichmann et al., 1988, Nature, 332:323-329; and Presta, 1992, Curr. Op. Struct. Biol., 2:593-596. The humanized antibody can also be a PRIMATIZED® antibody wherein the antigen-binding region of the antibody is derived from an antibody produced by immunizing macaque monkeys with the antigen of interest.

Transgenic animals (e.g., mice) that can, upon immunization, produce a full repertoire of human antibodies in the absence of endogenous immunoglobulin production can be produced. For example, homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice results in the production of human antibodies upon antigen challenge. See, for example, Jakobovits et al., 1993, Proc. Natl. Acad. Sci. USA, 90:2551; Jakobovits et al., 1993, Nature, 362:255-258; Bruggermann et al., 1993, Year in Immuno., 7:33. Human antibodies can also be derived from phage-display libraries, for example, as described in Hoogenboom et al., 1991, J. Mol. Biol., 227:381; or Marks et al., 1991, J. Mol. Biol., 222:581-597.

A “human antibody” is one that possesses an amino acid sequence corresponding to that of an antibody produced by a human and/or has been made using any of the techniques for making human antibodies as disclosed herein.

An “affinity matured” antibody is one having one or more alterations in one or more hypervariable regions that result in an improvement in the affinity of the antibody for antigen, compared to a parent antibody that does not possess those alteration(s). Preferred affinity matured antibodies will have nanomolar or even picomolar affinities for the target antigen. Affinity matured antibodies are produced by known procedures. See, for example, Marks et al., 1992, Bio/Technology 10:779-783, describing affinity maturation by VH and VL domain shuffling. Random mutagenesis of CDR and/or framework residues is described in Barbas et al., 1994, Proc. Nat. Acad. Sci. USA 91:3809-3813; Scier et al., 1995, Gene 169:147-155; Yelton et al., 1995, J. Immunol. 155:1994-2004; Jackson et al., 1995, J. Immunol. 154(7):3310-9; and Hawkins et al., 1992, J. Mol. Biol. 226:889-896.

“Antibody fragments” comprise only a portion of an intact antibody, generally including an antigen binding site of the intact antibody and thus retaining the ability to bind antigen. Examples of antibody fragments encompassed by the present definition include:
(i) the Fab fragment, having VL, CL, VH and CH1 domains having one interchain disulfide bond between the heavy and light chain;
(ii) the Fab′ fragment, which is a Fab fragment having one or more cysteine residues at the C-terminus of the CH1 domain;
(iii) the Fd fragment having VH and CH1 domains;
(iv) the FD′ fragment having VH and CH1 domains and one or more cysteine residues at the C-terminus of the CH1 domain;
(v) the Fv fragment having the VL and VH domains of a single arm of an antibody;
(vi) the dAb fragment that consists of a VH domain;
(vii) hingeless antibodies including at least VL, VH, CL, CH1 domains and lacking hinge region;
(viii) F(ab′)₂ fragments, a bivalent fragment including two Fab′ fragments linked by a disulfide bridge at the hinge region;
(ix) single chain antibody molecules (e.g. single chain Fv; scFv);
(x) “diabodies” with two antigen binding sites, comprising a heavy chain variable domain (VH) connected to a light chain variable domain (VL) in the same peptide chain;
(xi) single arm antigen binding molecules comprising a light chain, a heavy chain and a N-terminally truncated heavy chain constant region sufficient to form a Fc region capable of increasing the half life of the single arm antigen binding domain;
(xii) “linear antibodies” comprising a pair of tandem Fd segments (VH-CH1-VH-CH1) which, together with complementary light chain peptides, form a pair of antigen binding regions.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disorder, preventing metastasis, decreasing the rate of disease progression, amelioration or palliation of the disease state, and remission or improved prognosis.

TCCR

TCCR (WSX-1) is of the WS(G)XWS class of cytokine receptors with homology to the IL-12 β-2 receptor, G-CSFR, and IL-6 receptor. Highest homology is to the IL-12 β-2 receptor (26% identity). These receptors transduce a signal that controls growth and differentiation of cells, especially cells involved in blood cell growth and differentiation. Data presented in the examples below suggest that TCCR activation directly or indirectly induces suppression of autoimmune processes, including proliferation of CD8^{|} T-lymphocytes, or a Th1 response.

Suppression of autoimmune processes can occur through the induction of suppressor of cytokine signaling (SOCS) protein family members (Alexander et al., 2004, Ann. Rev. Immunol., 22:503), that may render T-cells non-responsive to other mitogenic stimuli. In particular, SOCS-3 is a protein that binds to the activation loop of Janus kinases, inhibiting kinase activity and thereby suppressing cytokine signaling (Masuhara et al., 1997, Biochem. Biophys. Res. Commun., 239: 439-446). It has been reported that the anti-inflammatory effect of some agents, such as peroxisome proliferator-activated receptor (PPAR)-gamma agonists (e.g., Rosiglitazone), function by inducing transcription of SOCS1 and SOCS3 (Park et al., 2003, J. Biol. Chem., 278: 14747-14752). Data presented in the examples below show that TCCR activation directly or indirectly induces expression of SOCS3.

Suppression of autoimmune processes also occurs through the induction of IL-10. IL-10 is a cytokine produced by activated T cells, B cells, monocytes, and keratinocytes. IL-10 inhibits the production of a number of cytokines, including IL-2, IL-3, IFN-γ, GM-CSF, and TNF. IL-10 plays a major role in limiting and terminating inflammatory responses (Moore et al., 2001, Ann. Rev. Immunol, 19: 683). Data presented in the examples below show that TCCR activation directly or indirectly induces expression of IL-10.

The amino acid sequence of human TCCR has been published (W097/44455 filed 23 May 1996) and is available from GenBank under accession number 4759327. This sequence is also described in Sprecher et al., 1998, Biochem. Biophys, Res. Commun. 246(1):82-90. The sequence of human TCCR (hTCCR) is 636 amino acids in length and is shown below in Table 1 (SEQ ID NO: 1). A signal peptide has been identified from amino acid residues 1 to 32, and a transmembrane domain from amino acid residues 517 to 538 of SEQ ID NO:1. N-glycosylation sites have been identified at residues 51-54, 76-79, 302-305, 311-314, 374-377, 382-385, 467-470, 563-566 and N-myristoylation sites at residues 107-112, 240-245, 244-249, 281-286, 292-297, 373-378, 400-405, 459-464, 470-475, 531-536 and 533-538. A prokaryotic membrane lipoprotein lipid attachment site is present at residues 522-532, and a growth factor and cytokine receptor family signature 1 at residues 41-54. There is also a region of significant homology with the second subunit of the receptor for human granulocyte-macrophage colony-stimulating factor (GM-CSF) at residues 183-191. TCCR binds with IL-27 subunit p28 at a cytokine receptor homology domain on TCCR at residues 41-230 of SEQ ID NO: 1. All hTCCR residues described are numbered according to the sequence of SEQ ID NO: 1.

In adults, hTCCR is most highly expressed in the thymus, but expression is also seen in peripheral blood leukocytes (PBL's), spleen, and weak expression in the lung. Fetal tissues exhibit weak TCCR expression in lung and kidney.

The amino acid sequence of murine TCCR (mTCCR) has been published (WO97/44455 filed 23 May 1996) and is available from GenBank under accession number 7710109. This sequence is also described in Sprecher et al., 1998, Biochem. Biophys, Res. Commun. 246(1):82-90. The sequence for mTCCR is 623 amino acids in length and is shown below in Table 1 (SEQ ID NO: 2). A signal peptide has been identified at amino acid residues 1 to 24, and a transmembrane domain from amino acid residues 514 to 532 of SEQ ID NO:2. N-glycosylation sites have been identified at residues, 46-49, 296-299, 305-308, 360-361, 368-371 and 461-464. Casein kinase II phosphorylation sites have been identified at residues 10-13, 93-96, 130-133, 172-175, 184-187, 235-238, 271-274, 272-275, 323-326, 606-609 and 615-618. A tyrosine kinase phosphorylation site has been identified at about residues 202-209. N-myristoylation sites have been identified at about residues 43-48, 102-107, 295-300, 321-326, 330-335, 367-342, 393-398, 525-530 and 527-532, and an amidation site at about residues 240-243. A prokaryotic membrane lipoprotein lipid attachment is present at about residues 516-526 and a growth factor and cytokine receptor family signature 1 is present at about residues 36-49. Regions of significant homology exist with human erythropoietin at about residues 14-51 and murine interleukin-5 receptor at residues 211-219. All mTCCR residues described are numbered according to the sequence of SEQ ID NO: 2.

TABLE 1
Human TCCR and Murine TCCR

| | | | | | |
|---|---|---|---|---|---|
| | 10 | 20 | 30 | 40 | |
| hTCCR | MRGGRGGPFW | LWPLPKLALL | PLLWVLFQRT | RPQGSAGPLQ | [SEQ ID NO:1] |
| mTCCR | -----MNRLR | VARLTPLELL | LSLMSLLLGT | RPHGSPGPLQ | [SEQ ID NO:2] |
| | 50 | 60 | 70 | 80 | |
| hTCCR | CYGVGPLGDL | NCSWEPLGDL | GAPSELHLQS | QKYRSNKTQT | |
| mTCCR | CYSVGPLGIL | NCSWEPLGDL | ETPPVLYHQS | QKYHPNRVWE | |
| | 90 | 100 | 110 | 120 | |
| hTCCR | VAVAAGRSWV | AIPREQLTMS | DKLLVWGTKA | GQPLWPPVFV | |
| mTCCR | VKVPSKQSWV | TIPREQFTMA | DKLLIWGTQK | GRPLWSSVSV | |
| | 130 | 140 | 150 | 160 | |
| hTCCR | NLETQMKPNA | PRLGPDVDFS | EDDPLEATVH | WAPPTWPSHK | |
| mTCCR | NLETQMKPDT | PQIFSQVDIS | EEATLEATVQ | WAPPVWPPQK | |
| | 170 | 180 | 190 | 200 | |
| hTCCR | VLICQFHYRR | CQEAAWTLLE | PELKTIPLTP | VEIQDLELAT | |
| mTCCR | ALTCQFRYKE | CQAEAWTRLE | PQLKTDGLTP | VEMQNLEPGT | |
| | 210 | 220 | 230 | 240 | |
| hTCCR | GYKVYGRCRM | EKEEDLWGEW | SPILSFQTPP | SAPKDVWVSG | |
| mTCCR | CYQVSGRCQV | ENGYP-WGEW | SSPLSFQTPF | LDPEDVWVSG | |
| | 250 | 260 | 270 | 280 | |
| hTCCR | NLCGTPGGEE | PLLLWKAPGP | CVQVSYKVWF | WVGGRELSPE | |
| mTCCR | TVCETSGKRA | ALLVWKDPRP | CVQVTYTVWF | GAGDITTTQE | |
| | 290 | 300 | 310 | 320 | |
| hTCCR | GITCCCSLIP | SGAEWARVSA | VNATSWEPLT | NLSLVCLDSA | |
| mTCCR | EVPCCKSPVP | AWMEWAVVSP | GNSTSWVPPT | NLSLVCLAPE | |
| | 330 | 340 | 350 | 360 | |
| hTCCR | SAPRSVAVSS | IAGSTELLVT | WQPGPGEPLE | HVVDWARDGD | |
| mTCCR | SAPCDVGVSS | ADGSPGIKVT | WKQGTRKPLE | YVVDWAQDGD | |
| | 370 | 380 | 390 | 400 | |
| hTCCR | PLEKLNWVRL | PPGNLSALLP | GNFTVGVPYR | ITVTAVSASG | |
| mTCCR | SLDKLNWTRL | PPGNLSTLLP | GEFKGGVPYR | ITVTAVYSGG | |
| | 410 | 420 | 430 | 440 | |
| hTCCR | LASASSVWGF | REELAPLVGP | TLWRLQDAPP | GTPAIAWGEV | |
| mTCCR | LAAAPSVWGF | REELVPLAGP | AVWRLPDDPP | GTPVVAWGEV | |
| | 450 | 460 | 470 | 480 | |
| hTCCR | PRHQLRGHLT | HYTLCAQSGT | SPSVCMNVSG | NTQSVTLPDL | |
| mTCCR | PRHQLRGQAT | HYTFCIQSRG | LSTVCRNVSS | QTQTATLPNL | |
| | 490 | 500 | 510 | 520 | |
| hTCCR | PWGPCELWVT | ASTIAGQGPP | GPILRLHLPD | NTLRWKVLPG | |
| mTCCR | HSGSFKLWVT | VSTVAGQGPP | GPDLSLHLPD | NRIRWKALPW | |
| | 530 | 540 | 550 | 560 | |
| hTCCR | ILFLWGLFLL | GCGLSLATS----G | RCYHLRHKVL | PRWVWEKVPD | |
| mTCCR | FLSLWGLLLM | GCGLSLASTRCLQA | RCLHWRHKLL | PQWIWERVPD | |
| | 570 | 580 | 590 | 600 | |
| hTCCR | PANSSSGQPH | MEQVPEAQPL | GDLPILEVEE | MEPPPVMESS | |
| mTCCR | PANSNSGQPY | IKEVSLPQPP | KDGPILEVEE | VELQPVVES- | |
| | 610 | 620 | 630 | 636 | |
| hTCCR | QPAQATAPLD | SGYEKHFLPT | PEELGLLGPP | RPQVLA | |
| mTCCR | ˜˜PKASAPIY | SGYEKHFLPT | PEELGLLV | (623) | |

IL-27

IL-27 is a ligand for TCCR (Pflanz et al., 2002 Immunity 16(6):779-790). IL-27 is a heterodimeric cytokine composed of EB13 (Epstein-Barr virus induced gene 3) and p28 protein subunits. p28 is a 4 helix bundle cytokine with three contact surfaces. A first contact surface binds EBI3, and comprises residues of the second and fourth alpha helix. A second contact surface binds TCCR in the region of the cytokine receptor homology domain and comprises residues of the first and third alpha helix. A third contact surface binds an IG domain, such as the IG domain found on gp130, and comprises residues at the end of the first helix and the beginning of the fourth.

The peptide sequence of human p28 (SEQ ID NO: 3) is 243 amino acids in length, whereas the peptide sequence of murine p28 (SEQ ID NO: 4) is 234 amino acids in length (Pflanz, NCBI Accession Number AAM34499). These sequences, shown below in Table 2, share 73% sequence identity.

TABLE 2
Human and Murine p28

| | | | | | |
|---|---|---|---|---|---|
| | 10 | 20 | 30 | 40 | |
| hp28 | MGQTAGDLGW | RLSLLLLPLLL | VQAGVWGFP | RPPGRPQLSL | (SEQ ID NO:3) |
| mp28 | MGQVTGDLGW | RLSLLLLPLLL | VQAGSWGFP | TDP----LSL | (SEQ ID NO:4) |
| | 50 | 60 | 70 | 80 | |
| hp28 | QELRREFTVS | LHLARKLLSE | VRGQAHRFAE | SHLPGVNLYL | |
| mp28 | QELRREFTVS | LYLARKLLSE | VQGYVHSFAE | SRLPGVNLDL | |
| | 90 | 100 | 110 | 120 | |
| hp28 | LPLGEQLPDV | SLTFQAWRRL | SDPERLCFIS | TTLQPFHALL | |
| mp28 | LPLGYHLPNV | SLTFQAWHHL | SDSERLCFLA | TTLRPFPAML | |
| | 130 | 140 | 150 | 160 | |
| hp28 | GGLGTQGRWT | NMERMQLWAM | RLDLRDLQRH | LRFQVLAAGF | |
| mp28 | GGLGTQGTWT | SSEREQLWAM | RLDLRDLHRH | LRFQVLAAGF | |
| | 170 | 180 | 190 | 200 | |
| hp28 | NLPEEEEEEE | EEEEEERKGL | -LP-GALGSALQ | GPAQVSWPQL | |
| mp28 | KCSKEEEDKE | EEEEEEEEEK | KLPLGALGGPNQ | VSSQVSWPQL | |
| | 210 | 220 | 230 | 243 | |
| hp28 | LSTYRLLHSL | ELVLSRAVRE | LLLLSKAGHS | VWPLGFPTLSPQP | |
| mp28 | LYTYQLLHSL | ELVLSRAVRD | LLLLSLPRRP | GSAWDS | (234) |
| hp28 | | | | | |
| mp28 | | | | | |

EBI3 has the structure of a soluble cytokine receptor and binds to a specific binding site on p28. Human EBI3 is 229 amino acids in length (Devergne et al., 1996, J. of Virology 70(2):1143-1153) and has a peptide sequence (SEQ ID NO: 5) shown below in Table 3.

TABLE 3
Human EBI3

| | | | |
|---|---|---|---|
| 10 | 20 | 30 | |
| MTPQLLLALV | LWASCPPCSG | RKGPPAALTL | (SEQ ID NO: 5) |
| 40 | 50 | 60 | |
| PRVQCRASRY | PIAVDCSWTL | PPAPNSTSPV | |
| 70 | 80 | 90 | |
| SFIATYRLGM | AARGHSWPCL | QQTPTSTSCT | |
| 100 | 110 | 120 | |
| ITDVQLFSMA | PYVLNVTAVH | PWGSSSSFVP | |
| 130 | 140 | 150 | |
| FITEHIIKPD | PPEGVRLSPL | AERQLQVQWE | |
| 160 | 170 | 180 | |
| PPGSWPFPEI | FSLKYWIRYK | RQGAARFHRV | |
| 190 | 200 | 210 | |
| GPIEATSFIL | RAVRPRARYY | VQVAAQDLTD | |
| 220 | 229 | | |
| YGELSDWSLP | ATATMSLGK | | |

TCCR/IL-27 Receptor Complex

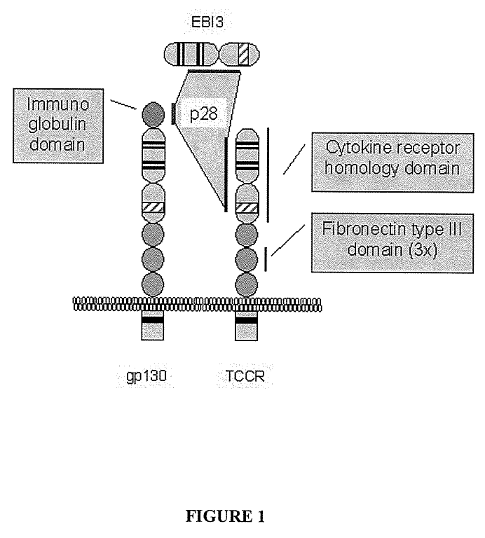

FIG. 1 shows the architecture of a TCCR/IL-27 receptor complex. The complete receptor for IL-27 contains gp130 and TCCR subunits. A cytokine receptor homology domain is present in gp130 at about residues 126-323 of SEQ ID NO: 6. Other homology domains present on gp130 include three fibronectin type III domains positioned at about residues 324-423, 424-518, and 519-614 of SEQ ID NO: 6, and an immunoglobulin domain at about residues 22-122. gp130 is also known to be a component of receptors for IL-6, IL-11, CNTF, LIF, CT1, and CLC (Hibi et al., 1990, Cell, 63(6):1149-1157). The amino acid sequence of gp130 (SEQ ID NO: 6) amino is shown below in Table 4.

TABLE 4
gp 130

| | | | |
|---|---|---|---|
| 10 | 20 | 30 | |
| MLTLQTWVVQ | ALFIFLTTES | TGELLDPCGY | (SEQ ID NO: 6) |
| 40 | 50 | 60 | |
| ISPESPVVQL | HSNFTAVCVL | KEKCMDYFHV | |
| 70 | 80 | 90 | |
| NANYIVWKTN | HFTIPKEQYT | IINRTASSVT | |
| 100 | 110 | 120 | |
| FTDIASLNIQ | LTCNILTFGQ | LEQNVYGITI | |
| 130 | 140 | 150 | |
| ISGLPPEKPK | NLSCIVNEGK | KMRCEWDGGR | |
| 160 | 170 | 180 | |
| ETHLETNFTL | KSEWATHKFA | DCKAKRDTPT | |
| 190 | 200 | 210 | |
| SCTVDYSTVY | FVNIEVWVEA | ENALGKVTSD | |
| 220 | 230 | 240 | |
| HINFDPVYKV | KPNPPHNLSV | INSEELSSIL | |
| 250 | 260 | 270 | |
| KLTWTNPSIK | SVIILKYNIQ | YRTKDASTWS | |
| 280 | 290 | 300 | |
| QIPPEDTAST | RSSFTVQDLK | PFTEYVFRIR | |
| 310 | 320 | 330 | |
| CMKEDGKGYW | SDWSEEASGI | TYEDRPSKAP | |
| 340 | 350 | 360 | |
| SFWYKIDPSH | TQGYRTVQLV | WKTLPPFEAN | |
| 370 | 380 | 390 | |
| GKILDYEVTL | TRWKSHLQNY | TVNATKLTVN | |
| 400 | 410 | 420 | |
| LTNDRYLATL | TVRNLVGKSD | AAVLTIPACD | |
| 430 | 440 | 450 | |
| FQATHPVMDL | KAFPKDNMLW | VEWTTPRESV | |
| 460 | 470 | 480 | |
| KKYILEWCVL | SDKAPCITDW | QQEDGTVHRT | |
| 490 | 500 | 510 | |
| YLRGNLAESK | CYLITVTPVY | ADGPGSPESI | |
| 520 | 530 | 540 | |
| KAYLKQAPPS | KGPTVRTKKV | GKNEAVLEWD | |
| 550 | 560 | 570 | |
| QLPVDVQNGF | IRNYTIFYRT | IIGNETAVNV | |
| 580 | 590 | 600 | |
| DSSHTEYTLS | SLTSDTLYMV | RMAAYTDEGG | |
| 610 | 620 | 630 | |
| KDGPEFTFTT | PKFAQGEIEA | IVVPVCLAFL | |
| 640 | 650 | 660 | |
| LTTLLGVLFC | FNKRDLIKKH | IWPNVPDPSK | |
| 670 | 680 | 690 | |
| SHIAQWSPHT | PPRHNFNSKD | QMYSDGNFTD | |
| 700 | 710 | 720 | |
| VSVVEIEAND | KKPFPEDLKS | LDLFKKEKIN | |
| 730 | 740 | 750 | |
| TEGHSSGIGG | SSCMSSSRPS | ISSSDENESS | |
| 760 | 770 | 780 | |
| QNTSSTVQYS | TVVHSGYRHQ | VPSVQVFSRS | |
| 790 | 800 | 810 | |
| ESTQPLLDSE | ERPEDLQLVD | HVDGGDGILP | |
| 820 | 830 | 840 | |
| RQQYFKQNCS | QHESSPDISH | FERSKQVSSV | |
| 850 | 860 | 870 | |
| NEEDFVRLKQ | QISDHISQSC | GSGQMKMFQE | |
| 880 | 890 | 900 | |
| VSAADAFGPG | TEGQVERFET | VGMEAATDEG | |
| 910 | 918 | | |
| MPKSYLPQTV | RQGGYMPQ | | |

IL-27 activation of TCCR induces expression of the major Th1-specific transcription factor, T-bet (Lucas et al., 2003, PNAS, 100:15047-52). The effects of TCCR activation are mediated by Stats (signal transducers and activators of transcription). Specifically, TCCR activation leads to phosphorylation of Stat1, Stat3, Stat4, and Stat5 (Lucas et al., 2003, supra). Data presented in the examples below suggest that TCCR activation directly or indirectly induces suppression of autoimmune processes, including proliferation of CD8⁺ T-lymphocytes, or a Th1 response.

TCCR and T-Lymphocyte Subtypes

As described above, members of the four helical bundle cytokine family (Bazan, J. F., 1990, Proc Natl Acad Sci USA, 87:6934-8) play a role in the expansion and terminal differentiation of T helper cells from a common precursor into distinct populations of Th1 and Th2 effector cells. (O'Garra., 1998, Immunity, 8:275-83.) IL-4 predominantly influences the development of Th2 cells, while IL-12 is a major factor in differentiation of Th1 cells. (Hsieh et al., 1993, Science, 260:547-9; Seder et al., 1993, Proc Natl Acad Sci USA, 90:10188-92; Le Gros et al., 1990, J Exp Med, 172:921-9; Swain et al., 1991, Immunol Rev, 123:115-44.) Accordingly, mice deficient in IL-4 (Kuhn et al., 1991, Science, 254:707-10), IL-4 receptor a chain (Noben-Trauth et al., 1997, Proc Natl Acad Sci USA, 94:10838-43), or the IL-4 specific transcription factor STAT6 (Shimoda et al., 1996, Nature, 380:630-3) are defective in Th2 responses, while mice deficient in IL-12 (Magram et al., 1996, Immunity, 4:471-81), IL-12 receptor (IL-12R) β1 chain (Wu et al., 1997, J Immunol, 159:1658-65), or the IL-12 specific transcription factor STAT4 (Kaplan et al., 1996, Nature, 382:174-7) have impaired Th1 responses.

Th1 and Th2 cell subtypes are derived from a common precursor, TH-0 cells. Cytokine release profiles from Th1 and Th2 cells affect selection of effector mechanisms and cytotoxic cells. 11-2 and interferon-gamma secreted by Th1 cells activate macrophages and cytotoxic cells, while Il-4, Il-5, Il-6, and Il-10 secreted by Th2 cells tends to increase production of eosinophils and mast cells, as well as enhance production of antibodies including IgE and decrease the function of cytotoxic cells. (Powrie et al., 1993, Immunol. Today, 14:270). Once established, a Th1 or Th2 response pattern is maintained by production of cytokines that generally inhibit cytokine production by cells of the other subset. For example, IL-4 inhibits production of interferon-gamma from Th1 clones, whereas interferon-gamma inhibits production of IL-4 from Th2 clones. (Mosmann et al., 1989, Adv. Immunol., 46:111-147; Mosmann et al., 1989, Annu. Rev. Immunol., 7:145-173). This negative feedback loop accentuates the production of polarized cytokine profiles during many immune responses.

Cytotoxic T-lymphocytes (CD8⁺) are able to rapidly destroy other cells. Cytotoxic T-lymphocytes use two major cytolytic pathways: the perforin-dependent exocytosis pathway and the Fas ligand/Fas pathway. Cytotoxic T-lymphocytes are also producers of pro-inflammatory cytokines such as interferon-gamma.

Overproduction of cytokines associated with a Th1 response or over-proliferation of CD8⁺ cytotoxic T-lymphocytes can lead to autoimmune disorders including allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), insulin-dependent diabetes mellitus, multiple sclerosis, pernicious anemia, psoriasis, rheumatoid arthritis, sarcoidosis, scleroderma, and systemic lupus erythematosus.

Studies detailed in the Examples below demonstrate greater proliferation of T-lymphocytes lacking TCCR compared with T-lymphocytes expressing TCCR, in response to non-specific T-lymphocyte stimulation (see Example 1). Further, it was surprisingly discovered that mice expressing TCCR were less susceptible to autoimmune disorders, such as experimental allergic encephalomyelitis (EAE), an animal model for multiple sclerosis, than were mice lacking TCCR (see Example 2).

These data suggest TCCR-mediated, direct or indirect, suppression of T-lymphocyte proliferation and Th1 mediated biological activities. Accordingly, agonists of TCCR can be used to inhibit T-cell proliferation and/or treat autoimmune disorders including multiple sclerosis and rheumatoid arthritis.

Autoimmune Disorders

As discussed above, over-proliferation of T-lymphocytes or over-production of cytokines produced by Th1 or Th2 cells leads to a host of medical disorders. For example, over-production of cytokines associated with a Th1 response or over-proliferation of CD8⁺ cytotoxic T-lymphocytes can lead to autoimmune disorders including allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), insulin-dependent diabetes mellitus, multiple sclerosis, pernicious anemia, psoriasis, rheumatoid arthritis, sarcoidosis, scleroderma, and systemic lupus erythematosus.

Multiple Sclerosis is an autoimmune demyelinating disorder that is believed to be T lymphocyte dependent. MS generally exhibits a relapsing-remitting course or a chronic progressive course. The etiology of MS is unknown, however, viral infections, genetic predisposition, environment, and autoimmunity all appear to contribute to the disorder. Lesions in MS patients contain infiltrates of predominantly T lymphocyte mediated microglial cells and infiltrating macrophages. CD4⁺ T lymphocytes are the predominant cell type present at these lesions. The hallmark of the MS lesion is plaque, an area of demyelination sharply demarcated from the usual white matter seen in MRI scans. Histological appearance of MS plaques varies with different stages of the disease. In active lesions, the blood-brain barrier is damaged, thereby permitting extravasation of serum proteins into extracellular spaces. Inflammatory cells can be seen in perivascular cuffs and throughout white matter. CD4⁻ T-cells, especially Th1, accumulate around postcapillary venules at the edge of the plaque and are also scattered in the white matter. In active lesions, up-regulation of adhesion molecules and markers of lymphocyte and monocyte activation, such as IL2-R and CD26 have also been observed. Demyelination in active lesions is not accompanied by destruction of oligodendrocytes. In contrast, during chronic phases of the disease, lesions are characterized by a loss of oligodendrocytes and hence, the presence of myelin oligodendrocyte glycoprotein (MOG) antibodies in the blood.

Various well-accepted animal models exist for autoimmune disorders. By way of example, EAE (experimental allergic encephalomyelitis) is a T cell mediated autoimmune disorder characterized by T cell and mononuclear cell inflammation and subsequent demyelination of axons in the central nervous system. EAE is generally considered to be a relevant animal model for MS in humans. (See, for example, Bolton, C., 1995, Multiple Sclerosis, 143.) Agents, such as candidate TCCR agonists, can be analyzed for T cell stimulatory or inhibitory activity against immune mediated demyelinating disorders, for example, using the protocol described in Current Protocols in Immunology, units 15.1 and 15.2; edited by Coligan et al., National Institutes of Health, Published by John Wiley & Sons, Inc. See also models for myelin disease in which oligodendrocytes or Schwann cells are grafted into the central nervous system, for example, as described in Duncan et al., 1997, Molec. Med. Today, 554-561.

An animal model for arthritis is collagen-induced arthritis. See, for example, McIndoe et al., 1999, Proc. Natl. Acad. Sci. USA, 96:2210-2214. This model shares clinical, histological, and immunological characteristics of human autoimmune rheumatoid arthritis and is an acceptable model for human autoimmune arthritis. Mouse and rat models are characterized by synovitis, erosion of cartilage, and subchondral bone. Collagen-induced arthritis shares many features with rheumatoid arthritis in humans including lymphocytic infiltration and synovial membrane hypertrophy. See, for example, McIndoe et al., 1999, Proc. Natl. Acad. Sci. USA, 96:2210-2214. Potential agonists of TCCR can be analyzed for activity against autoimmune arthritis using these models, for example, using the protocols described in Current Protocols in Immunology, units 15.5; edited by Coligan et al., National Institutes of Health, Published by John Wiley & Sons, Inc. See also the model using a monoclonal antibody to CD 18 and VLA-4 integrins described in Issekutz, A. C. et al., Immunology (1996) 88:569.

An animal model for skin allograft rejection is a means of testing the ability of T cells to mediate in vivo tissue destruction that is indicative of, and a measure of, their role in anti-viral and tumor immunity. The most common and accepted models use murine tail-skin grafts. Repeated experiments have shown that skin allograft rejection is mediated by T cells, helper T cells and killer-effector T cells, and not antibodies. See, for example, Auchincloss and Sachs, 1998, In: Fundamental Immunology, 2nd ed., W. E. Paul ed., Raven Press, NY, at pages 889-992. A suitable procedure is described in detail in Current Protocols in Immunology, unit 4.4; edited by Coligan et al., 1995, National Institutes of Health, Published by John Wiley & Sons, Inc. Other transplant rejection models that can be used to screen candidate TCCR agonists include the allogeneic heart transplant models described, for example, by Tanabe et al., 1994, Transplantation, 58:23 and Tinubu et al., 1994, J. Immunol., 4330-4338.

Agonists of TCCR

Agonists of TCCR are molecules that enhance or stimulate a biological activity of a native sequence TCCR peptide disclosed herein. Suitable agonist molecules specifically include agonist antibodies, including humanized antibodies, or fragments of agonist antibodies, including Fab, Fab′, Fd, Fd′, Fv, dAb, hingeless antibodies, F(ab′)2 fragments, single chain antibody molecules, diabodies, single arm antigen binding molecules, and linear antibodies, amino acid sequence variants or fragments of native polypeptides, peptides, small molecules, and the like.

Suitable agonists of TCCR also include peptide fragments of TCCR, the TCCR extracellular domain, and TCCR variants having at least about 80% amino acid sequence identity with the amino acid sequence of:

(a1) residue 1 or about 33 to 636 of the human TCCR peptide of SEQ ID NO:1;

(a2) residue 1 or about 25 to 623 of the murine TCCR peptide of SEQ ID NO:2;

(b1) X3 to 636 of the human TCCRpeptide of SEQ ID NO:1, where X3 is any amino acid residue 27 to 37 of SEQ ID NO:1;

(b2) X4 to 623 of the murine TCCR peptide of SEQ ID NO:2, where X4 is any amino acid residue from 20 to 30 of SEQ ID NO:2;

(c1) 1 or about 33 to X1, where X1 is any amino acid residue from residue 512 to residue 522 of SEQ ID NO:1;

(c2) 1 or about 25 to X2, where X2 is any amino acid residue from residue 509 to 519 of SEQ ID NO:2;

(d1) X5 to 636, where X5 is any amino acid from residue 533 to 543 of SEQ ID NO:1;

(d2) X6 to 623, where X6 is any amino acid from residue 527 to 537 of SEQ ID NO:2; or

(e) another specifically derived fragment of the amino acid sequences of SEQ ID NO:1 and SEQ ID NO:2.

Agonists of TCCR include, for example, TCCR peptides where one or more amino acid residue is added, or deleted, at the N— and/or C-terminus, or within one or more internal domains, of the sequence of SEQ ID NO:1 and SEQ ID NO:2.

Agonists of TCCR include native sequence IL-27, EBI3, p28, variants and fragments thereof having biological activities normally associated with the IL-27 heterodimer. For example, agonists of TCCR include IL-27 variants having at least 80%, 90%, 95%, or 99% sequence identity with the native sequence components of IL-27. Agonists of TCCR also include p28 variants having at least 73%, 75%, 80%, 90%, 95%, or 99% sequence identity with native sequence human p28 (SEQ ID NO: 3) or murine p28 (SEQ ID NO: 4). Agonists of TCCR include portions of p28 capable of binding TCCR and gp130. By way of example, agonists of TCCR include p28 variants having at least 73%, 75%, 80%, 90%, 95%, or 99% sequence identity with native sequence human p28 (SEQ ID NO: 3) or murine p28 (SEQ ID NO: 4) and capable of binding TCCR and gp130. Agonists of TCCR include p28 peptide variants containing residues from the first and third alpha helices of p28, believed to bind TCCR in the region of the cytokine receptor homology domain found on TCCR, and residues at the end of the first helix and the beginning of the fourth helix, believed to bind the IG domain found on gp130.

Agonists of TCCR include, for example, molecules that are able to bind and activate TCCR. Agonists of TCCR also include molecules that are able to cause TCCR to form a homodimer and/or those molecules that are able to cause TCCR and gp130 to form a heterodimer. For example, antibodies to TCCR may be able to cause TCCR to form a homodimer. As a further example, bivalent antibodies specific to both TCCR and gp130 may be able to cause TCCR and gp130 to form a heterodimer.

Methods for identifying agonists or antagonists of a TCCR peptide may include contacting a TCCR peptide with a candidate agonist or antagonist molecule and measuring a detectable change in one or more biological activities associated with the TCCR peptide. For example, agonists are identified by contacting a cell expressing TCCR peptide with a candidate, then analyzing the contacted cells for a biological activity of TCCR, such as phosphorylation of Stat1, Stat3, Stat4, or Stat5, using Western-blot or another suitable assay. Agonists of TCCR can also be identified by contacting cells, such as Ba/F3 cells, engineered to express TCCR peptide with a candidate agonist, then analyzing the contacted cells for proliferation, for example by measuring [³H] labeled thymidine incorporation or another suitable assay.

Monoclonal Antibodies

Many techniques for producing monoclonal antibodies are known. In one method, for example, mice such as Balb/c, are immunized with TCCR or a portion thereof as an immunogen, emulsified in complete Freund's adjuvant, and injected subcutaneously or intraperitoneally in an amount from 1 -100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-TCCR antibodies.

After a suitable antibody titer has been detected, animals “positive” for anti-TCCR antibodies can be injected with a final intravenous injection of the immunogen. Three to four days later, the mice are sacrificed and spleen cells are harvested. The spleen cells are then fused (using 35% polyethylene glycol) to a selected murine myeloma cell line such as P3X63AgU.1, available from ATCC, No. CRL 1597. The fusions generate hybridoma cells that can then be plated in 96 well tissue culture plates containing HAT (hypoxanthine, aminopterin, and thymidine) medium to inhibit proliferation of non-fused cells, myeloma hybrids, and spleen cell hybrids.

Selected hybridoma cells can be screened in an ELISA or other suitable assay, for reactivity against TCCR. Positive hybridoma cells can be injected intraperitoneally into, for example, syngeneic Balb/c mice to produce ascites containing the anti-TCCR monoclonal antibodies. Alternatively, the hybridoma cells can be grown, for example, in tissue culture flasks or roller bottles. Purification of monoclonal antibodies produced in the ascites can be accomplished using ammonium sulfate precipitation, followed by gel exclusion chromatography, or other suitable method. Alternatively, affinity chromatography based upon binding of antibody to protein A or protein G can be employed.

TCCR −/− Mice

“Knock out” mice have been constructed that do not express TCCR (TCCR −/−). Such mice may be prepared, for example through homologous recombination between the endogenous gene encoding TCCR and altered genomic DNA encoding the same peptide introduced into an embryonic cell of the animal.

For example, cDNA encoding a particular peptide can be used to clone genomic DNA encoding that peptide in accordance with established techniques. A portion of the genomic DNA encoding a particular peptide can be deleted or replaced with another gene, such as a gene encoding a selectable marker that can be used to monitor integration. Typically, several kilobases of unaltered flanking DNA (both at the 5′ and 3′ ends) are included in the vector (see Thomas et al., 1987, Cell, 51:503 for a description of homologous recombination vectors). The vector is introduced into an embryonic stem cell line (such as by electroporation) and cells in which the introduced DNA has homologously recombined with the endogenous DNA are selected. See, for example, Li et al., 1992, Cell, 69:915. The selected cells are then injected into a blastocyst of an animal (such as a mouse or rat) to form aggregation chimeras, as described, for example, in Bradley et al., 1987, In: Teratocarcinomas and Embryonic Stem Cells: A Practical Approach, E. J. Robertson, ed. (IRL, Oxford), pp. 113-152. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA.

A description of the creation of TCCR −/− mice used in the examples below is found in WO0129070 (de Sauvage et al.) and Chen et al., 2000, Nature, 407:916, the contents of which are hereby incorporated by reference.

Compositions and Treatment

Agonists of TCCR useful in the treatment of autoimmune disorders include, without limitation, proteins, antibodies, fragments and variants, small organic molecules, peptides, phosphopeptides, and the like, that modulate immune function, for example, T cell proliferation/activation, lymphokine release, or immune cell infiltration. In particular, agonists of TCCR described herein are useful to suppress, diminish, or reduce T-lymphocyte proliferation, T-lymphocyte cytokine release, and autoimmune disorders.

TCCR agonists can be identified by any of the screening assays discussed above and/or by any other known screening techniques.

TCCR agonists of the present invention can be formulated according to known methods to prepare useful compositions, whereby the TCCR agonist is combined with an acceptable carrier. Formulations are prepared for storage by mixing the TCCR agonists having the desired degree of purity with optional acceptable carriers, excipients, or stabilizers, in the form of lyophilized formulations or aqueous solutions. See, for example, Remington: The Science and Practice of Pharmacy 20th ed. Gennaro Ed. (2000). Acceptable carriers, excipients, or stabilizers are nontoxic to recipients at the dosages and concentrations employed, and include buffers such as phosphate, citrate, and other organic acids; antioxidants including ascorbic acid; low molecular weight (less than about 10 residues) peptides; proteins, such as serum albumin, gelatin, or immunoglobulins; hydrophilic polymers such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, PLURONICS®, or PEG.

Formulations to be used for in vivo administration must be sterile. This is readily accomplished by filtration through sterile filtration membranes, prior to or following lyophilization and reconstitution.

Compositions herein generally are placed into a container having a sterile access port, for example, an intravenous solution bag or vial having as stopper pierceable by a hypodermic injection needle.

The route of administration is in accord with known methods, such as injection or infusion by intravenous, intraperitoneal, intracerebral, intramuscular, intraocular, intraarterial, or intralesional routes, topical administration, or by sustained release systems. The route of administration may also include in vivo expression as a result of transfection with a suitable vector, such as an adenoviral vector.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” in Toxicokinetics and New Drug Development, Yacobi et al, Eds., Pergamon Press, New York 1989, pp. 42-96.

When in vivo administration of a TCCR agonist is employed, normal dosage amounts may vary from about 10 ng/kg to up to 100 mg/kg of mammal body weight or more per day, for example about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatments and different disorders, and that administration intended to treat a specific organ or tissue, may necessitate delivery in a manner different from that to another organ or tissue.

Where sustained-release administration of TCCR agonists is desired in a formulation with release characteristics suitable for the treatment of any disease or disorder requiring administration of the TCCR agonists, microencapsulation of the TCCR agonists is contemplated. Microencapsulation of recombinant proteins for sustained release has been successfully performed with human growth hormone (rhGH), interferon-alpha, -beta, -gamma, interleukin-2, and MN rgp120. See, for example, Johnson et al., 1996, Nat. Med. 2: 795-799; Yasuda, 1993, Biomed. Ther., 1221-1223; Hora et al., 1990, Bio/Technology, 755-758; Cleland, 1995, “Design and Production of Single Immunization Vaccines Using Polylactide Polyglycolide Microsphere Systems” in Vaccine Design: The Subunit and Adjuvant Approach, Powell and Newman, eds., (Plenum Press: New York), pp. 439-462; WO 97/03692, WO 96/40072, WO 96/07399, and U.S. Pat. No. 5,654,010.

Sustained-release formulations of TCCR agonists may be developed using poly-lactic-coglycolic acid (PLGA), a polymer exhibiting a strong degree of biocompatibility and a wide range of biodegradable properties. The degradation products of PLGA, lactic and glycolic acids, are cleared quickly from the human body. Moreover, the degradability of this polymer can be adjusted from months to years depending on its molecular weight and composition. For further information see Lewis, “Controlled Release of Bioactive Agents from Lactide/Glycolide polymer,” in Biogradable Polymers as Drug Delivery Systems M. Chasin and R. Langeer, editors (Marcel Dekker: New York, 1990), pp. 1-41.

### EXAMPLES

The present invention may be better understood with reference to the following examples. These examples are intended to be representative of specific embodiments of the invention, and are not intended as limiting the scope of the invention.

### Example 1

### TCCR-Mediated Suppression of T-Cell Response

The effect of TCCR activity on T-cell response was tested by analysis of induced T-cell proliferation of wild-type (TCCR +/+) and knock-out (TCCR −/−) splenocytes. The T-cell receptor associates with CD3 to form a T-cell receptor complex. Anti-CD3 antibodies at a sufficient dose non-specifically stimulate proliferation of T-cells normally associated with the interaction of T-cell receptor complex and MHC class TI molecules (CD4) of an antigen-presenting cell (APC).

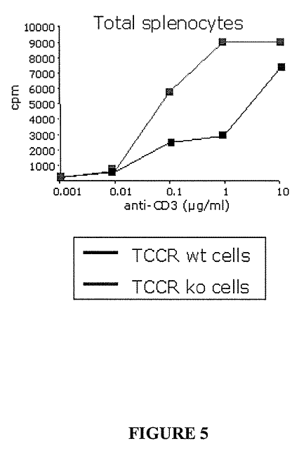

Proliferation of wild-type (TCCR +/+) and knock-out (TCCR −/−) mixed lymphocytes, isolated CD4⁺ T cells, and isolated CD8⁺ T cells were stimulated by anti-CD3 antibody (BD Pharmingen, San Diego, Calif., clone 145-2c11). Cells were grown for three days in a humidified CO₂ incubator and proliferation was measured by [³H]-thymidine incorporation as measured during the last 8-16 hours of the assay. Surprisingly, anti-CD3 antibody induced proliferation of mixed lymphocytes obtained from knock-out mice (TCCR −/−) was significantly greater than that of lymphocytes obtained from wild-type (TCCR +/+) lymphocytes at submaximal doses of anti-CD3, as shown in Table 5 and in FIG. 5. This data suggests a protective effect of TCCR activity, for example, suppressing proliferation of stimulated T-cells, and that stimulation of TCCR with an agonist might be useful to directly or indirectly suppress T-cell response, such as T-cell proliferation.

TABLE 5

| | | |
|---|---|---|
| anti-CD3 | TCCR wt | TCCR ko |

| (μg/ml) | Average | Stand. Dev. | Average | Stand. Dev. |
|---|---|---|---|---|
| 10.0 | 7284 | 771 | 9012 | 1396 |
| 1.0 | 2853 | 1016 | 9029 | 1410 |
| 0.1 | 2444 | 809 | 5756 | 721 |
| 0.01 | 528 | 266 | 651 | 77 |
| 0.001 | 180 | 63 | 222 | 37 |
| 0.0001 | 133 | 20 | 255 | 129 |
| 0 | 127 | 68 | 323 | 91 |

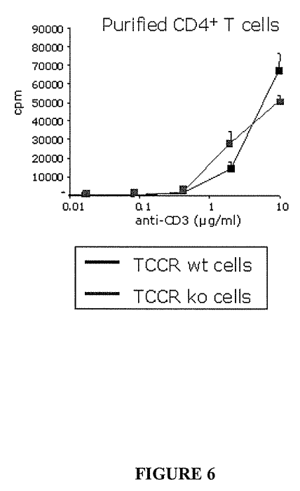

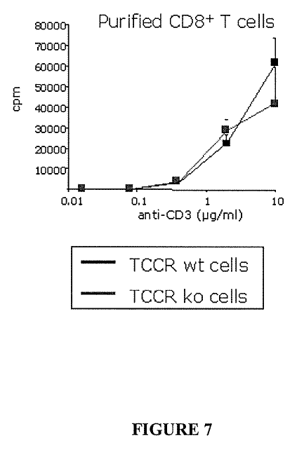

However, anti-CD3 antibody induced proliferation of isolated CD4⁺ T cells and isolated CD8⁺ T cells was not significantly different between wild-type and knock-out cells, as shown in FIG. 6 (Table 6) and FIG. 7 (Table 7) respectively. This data suggests that IL-27, a ligand for TCCR, is produced by lymphocytes other than CD4⁺ T cells and CD8⁺ T cells.

TABLE 6

| | | |
|---|---|---|
| anti-CD3 | TCCR wt | TCCR ko |

| (μg/ml) | Average | Stand. Dev. | Average | Stand. Dev. |
|---|---|---|---|---|
| 10.0 | 67865 | 8381 | 50977 | 2812 |
| 2.0 | 14540 | 3465 | 28228 | 6076 |
| 0.4 | 804 | 340 | 1330 | 834 |
| 0.08 | 84 | 9 | 78 | 21 |
| 0.016 | 111 | 3 | 84 | 18 |
| 0.0032 | 88 | 16 | 85 | 11 |
| 0.00064 | 121 | 61 | 71 | 3 |
| 0 | 149 | 78 | 86 | 21 |

TABLE 7

| | | |
|---|---|---|
| anti-CD3 | TCCR wt | TCCR ko |

| (μg/ml) | Average | Stand. Dev. | Average | Stand. Dev. |
|---|---|---|---|---|
| 10.0 | 61657 | 11913 | 42067 | 17014 |
| 2.0 | 22778 | 3613 | 28727 | 5408 |
| 0.4 | 3362 | 984 | 3862 | 1973 |
| 0.08 | 139 | 58 | 227 | 53 |
| 0.016 | 170 | 43 | 155 | 59 |
| 0.0032 | 125 | 66 | 136 | 15 |
| 0.00064 | 141 | 35 | 112 | 18 |
| 0 | 142 | 58 | 127 | 64 |

Next, the proliferation of CD4⁺ and CD8⁺ cells in response to anti-CD3 antibody stimulation was measured in a CFSE (carboxyfluorescein diacetate, succinimidyl ester) labeling assay. CFSE labeling allows the number of cell divisions to be monitored as labeled cells lose 50% of their fluorescence intensity after each cell division. Wild-type (TCCR +/+) and knock-out (TCCR −/−) mixed lymphoocyte cell suspensions were labeled with CFSE to create a concentration of 0.5 μM CFSE (Sigma, St. Louis, Mo.) in the cell suspension. The cell suspensions were then incubated for 10 minutes at 37° C. After labeling, FCS was added to 5% final concentration and the cells were immediately centrifuged and washed with ice-cold PBS. Proliferation of wild-type (TCCR +/+) and knock-out (TCCR −/−) mixed lymphocytes cells was stimulated by anti-CD3 antibody (BD pharmingen, San Diego, Calif., clone 145-2c11) at a concentration of 2.5 μg/ml.

The cells were then incubated at 37° C. for 2 days. At that point, the cells were labeled with markers for CD4^{|} and CD8^{|} (CD4-Cychrome or CD8-Cychrome) and analyzed by flow cytometry.

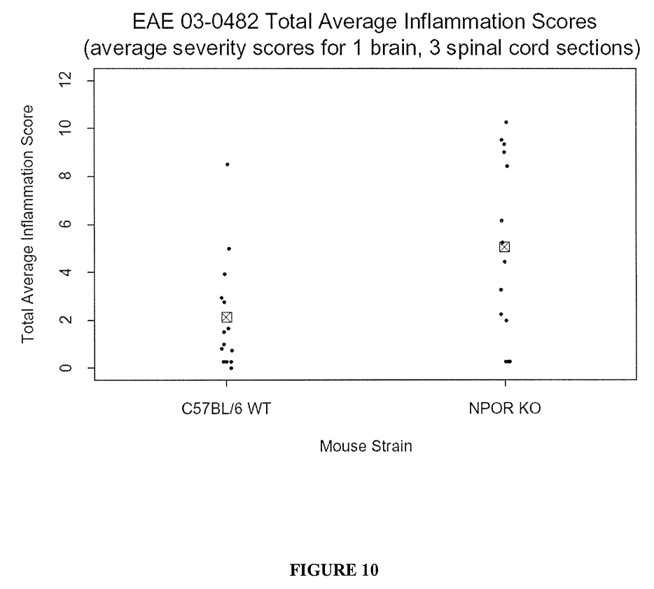

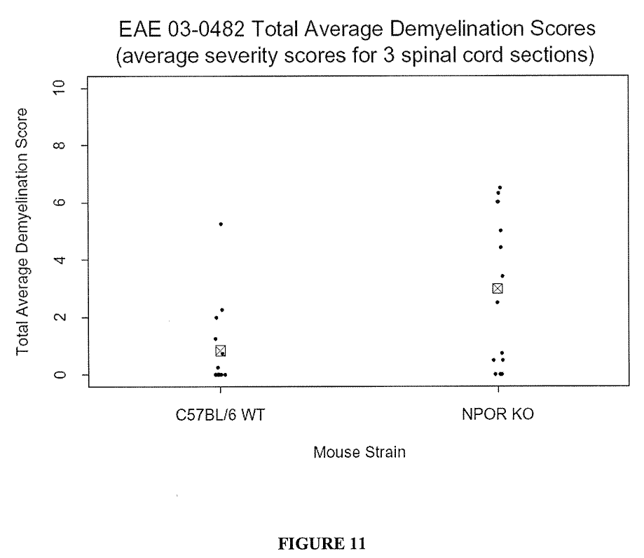

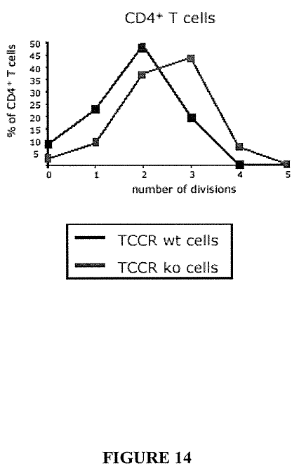

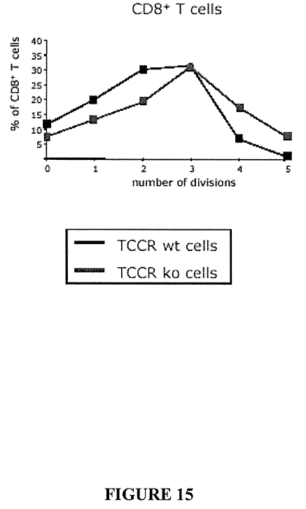

The data below show that both CD4⁺ as well as CD8⁺ positive T cells are hyperproliferative in TCCR knock-out cells (see Table 8). FIGS. 10 and 11 depict the number of cells that have undergone 0, 1, 2, 3, 4, or 5 cell divisions during the incubation period. For both CD4⁺ and CD8⁺ T cells, more cells have undergone 3, 4, and 5 divisions in the knock-out than in the wild-type (i.e. the line for the knock-out cells is shifted to the right in both CD4⁺ cells (FIG. 14) and CD8⁺ cells (FIG. 15)).

TABLE 8

| Number of | CD4+ TCCR | CD4+ TCCR | CD8+ TCCR | CD8+ TCCR |
|---|---|---|---|---|
| Divisions | wt | ko | wt | ko |
| 0 | 8.5 | 2.66 | 11.47 | 7.37 |
| 1 | 22.75 | 8.97 | 20.02 | 13.25 |
| 2 | 48.86 | 37.44 | 30.19 | 19.12 |
| 3 | 19.8 | 43.75 | 31.49 | 31.11 |
| 4 | 0.33 | 7.43 | 6.67 | 17.45 |
| 5 | 0.19 | 0.51 | 0.73 | 7.43 |
| 6 | 0.14 | 0.09 | 0.08 | 1.84 |
| 7 | 0.14 | 0.09 | 0.16 | 0.81 |
| 8 | 0 | 0.09 | 0.08 | 0.98 |
| 9 | 0 | 0 | 0 | 0 |

### Example 2

### Mice Expressing TCCR Are Less Susceptible to EAE

Experimental allergic encephalomyelitis (EAE) is an autoimmune disorder of the CNS that serves as an animal model for multiple sclerosis (MS). Similar to MS, EAE is a demyelinating disorder where immune-mediated damage to myelin results in observable symptoms. EAE is believed to be mediated by both CD4⁻ Th1 cells (Fife et al., 2001, J. of immun., 166:7617-7624) and CD8+cytotoxic T-lymphocytes (CTLs) (Huseby et al., 2001, J. Exp. Med., 194(5):669-676). To examine the effect of TCCR on EAE, clinical progression of EAE was examined in wild-type mice expressing TCCR (TCCR +/+) and knock-out mice lacking TCCR (TCCR −/−). As shown below, mice expressing TCCR were less susceptible to the CD4⁺ Th1 and CD8⁺ mediated disorder EAE than were mice lacking TCCR.

Knock-out TCCR −/− mice were generated as described in WO0129070 (de Sauvage et al.) and back-crossed onto the C57BL/6 background and bred from N12 founders. Wild-type TCCR +/+controls were C57BL/6 mice purchased from The Jackson Laboratory (Bar Harbor, Me.).

MOG Induced EAE

MOG 35-55 peptide having an amino acid sequence of MEVGWYRSPFSRVVHLYRNGK (SEQ ID NO: 7) was synthesized using 9-fluorenylmethoxycarbonyl chemistry on a Rainin Quartet automated peptide synthesizer (Rainin, Oakland, Calif.). The peptide was cleaved from the resin and purified by using preparative reversed phase HPLC with water/acetonitrile/0.1% TFA gradients in the mobile phase. The identity of the peptide was confirmed by electrospray mass spectrometry.

Wild-type TCCR +/+and knock-out TCCR −/− mice were immunized intradermally with 200 μl of an emulsion containing 200 μg of MOG 35-55 peptide in 100 μl of PBS and 100 μl of CFA (complete Freund adjuvant) to induce EAE on day 0. CFA was prepared by mixing IFA (incomplete Freund adjuvant) (Difco-BD Diagnostic Systems, Sparks, Md.) with dead and dessicated M. tuberculosis H37A (Difco-BD Diagnostic Systems, Sparks, Md.) to a concentration of 8 mg/ml M. tuberculosis (each mouse received 800 μg of dead M. tuberculosis as a component of the CFA). On Day 0 and again on Day 2, each mouse was injected intraperitoneally with 200 ng of Pertussis toxin (List Biological Laboratories, Campbell, Calif.) in 100 μl of PBS, to aid in penetrating the blood brain barrier. The doses of components received are summarized in Table 9 below.

TABLE 9

| | |
|---|---|
| Day 0 | 200 μg of MOG 35-55 in 100 μl of PBS |
| | and |
| | 100 μl of CFA intradermally. |
| | 200 ng Pertussis toxin in 100 μl of PBS |
| | intraperitoneally. |
| Day 1 | None |
| Day 2 | 200 ng Pertussis toxin in 100 μl of PBS |
| | intraperitoneally. |

MBP Induced EAE

Ac 1-11 peptide having an amino acid sequence of ASQKRPSQRHG (SEQ ID NO: 8) was synthesized using 9-fluorenylmethoxycarbonyl chemistry on a Rainin Quartet automated peptide synthesizer (Rainin, Oakland, Calif.). The peptide was cleaved from the resin and purified by using preparative reversed phase HPLC with water/acetonitrile/0.1% TFA gradients in the mobile phase. The identity of the peptide was confirmed by electrospray mass spectrometry.

Wild-type TCCR +/+and knock-out TCCR −/− mice were immunized intradermally with 10 μg of Ac 1-11 peptide (ASQKRPSQRHG), a component of myelin basic protein, in 100 μl of CFA (complete Freund adjuvant) to induce EAE on day 0. As discussed above for MOB-induced EAE, CFA was prepared by mixing IFA (incomplete Freund adjuvant) with M. tuberculosis H37A (dead and desiccated) to the concentration of 8 mg/ml M. tuberculosis (each mouse received 800 μg of dead M. tuberculosis as a component of the CFA). On Day 2 and again on Day 3, each mouse was injected intraperitoneally with 200 ng of Pertussis toxin in 100 μl of PBS, to aid in penetrating the blood brain barrier. The doses of components received are summarized in Table 10 below.

TABLE 10

| | |
|---|---|
| Day 1 | 10 μg of Ac 1-11 peptide in 100 μl of CFA |
| | subcutaneously. |
| Day 2 | 200 ng Pertussis toxin in 100 μl of PBS |
| | intraperitoneally. |
| Day 3 | 200 ng Pertussis toxin in 100 μl of PBS |
| | intraperitoneally. |

All mice were evaluated for clinical disease 3 times per week starting on day 1. Mice that reached disease grade 4 were evaluated daily. Any animal at grade 5 was euthanized. Those that failed to improve to grade 3 or less in 5 days were euthanized. The clinical grading system used is shown in Table 11 below:

TABLE 11
Clinical Grading System

| | |
|---|---|
| Grade 0 | Normal mouse, no overt signs of disease. |
| Grade 1 | Limp tail (complete flaccidity of the tail, |
| | and absence of curling at the tip of the tail |
| | when mouse is picked up), or |
| | Hind limb weakness (observed as a |
| | wadding gait, the objective sign being |
| | that, in walking, mouse hind limbs fall |
| | through wire cage tops) but not both. |
| Grade 2 | Limp tail and hind limb weakness. |
| Grade 3 | Partial hind limb paralysis (mouse can no |
| | longer use hind limbs to maintain rump |
| | posture or walk but can still move one or |
| | both limbs to some extent). |
| Grade 4 | Complete hind limb paralysis (total loss of |
| | movement in hind limbs; mouse drags |
| | itself only on forelimbs). |
| Grade 5 | Moribund state, death by EAE, sacrifice |
| | for humane reasons. |

On day 40, all remaining animals were sacrificed and brains and spinal cords were dissected out for histological analysis. Brains were sectioned, one section from each of four levels for each brain, and stained with H&E (hematoxylin and eosin stain, Sigma, St. Louis, Mo.) in order to evaluate inflammation. Spinal cords were sectioned, four sections from each of three different levels for each spinal cord, and stained with H&E in order to evaluate inflammation and Luxol Fast Blue (VWR Scientific, St. Paul, Minn.) in order to evaluate demyelination. For each slide, the highest score and an average score (average of all the sections on each slide) for both inflammation and demyelination were reported. The inflammation grading system used is shown in Table 12 below:

TABLE 12
Inflammation Grading System

| | |
|---|---|
| Grade 0 | No significant findings. |
| Grade 1 | Minimal-mild perivascular inflammation. |
| Grade 2 | Mild-moderate inflammation that extends |
| | beyond vessels. |
| Grade 3 | Moderate to Marked Inflammation that |
| | extends well beyond vessels. |
| Grade 4 | Severe inflammation that involves much |
| | of the neuropil. |

The demyelination grading system used is shown in Table 13 below:

TABLE 13
Demyelination Grading System

| | |
|---|---|
| Grade 0 | No significant findings |
| Grade 1 | Minimal |
| Grade 2 | Mild |
| Grade 3 | Moderate |
| Grade 4 | Marked |

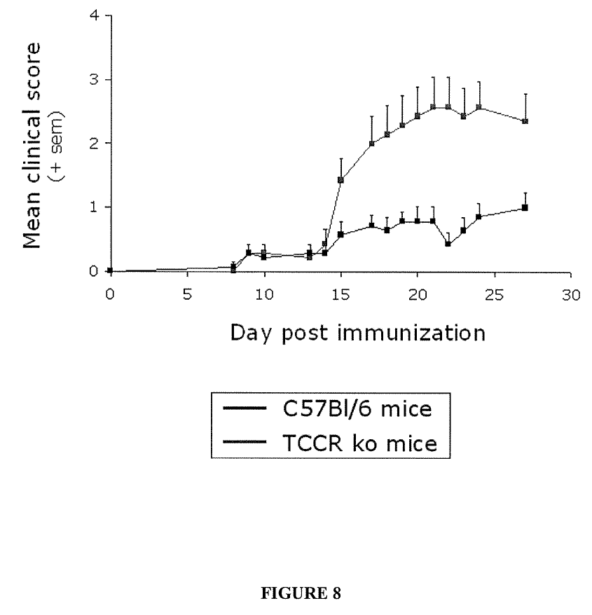

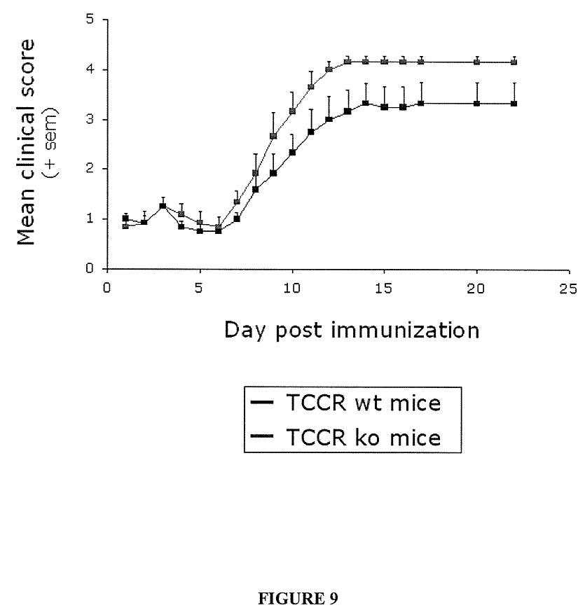

Clinical progression of MOG (myelin oligodendrocyte glycoprotein) induced EAE (experimental allergic encephalomyelitis) in wild-type (TCCR +/+) was less severe than induced EAE in knock-out (TCCR −/−) mice (See FIG. 8). Similarly, clinical progression of MBP (myelin basic protein) induced EAE in wild-type (TCCR +/+) mice was less severe than induced EAE in knock-out (TCCR −/−) mice (See FIG. 9).

As shown in FIGS. 8 and 9, animals lacking TCCR (TCCR −/−) showed more severe clinical symptoms of EAE, whereas mice expressing TCCR (wt) showed less severe symptoms and progression, suggesting a protective effect of TCCR activity against autoimmune disorders, such as EAE.

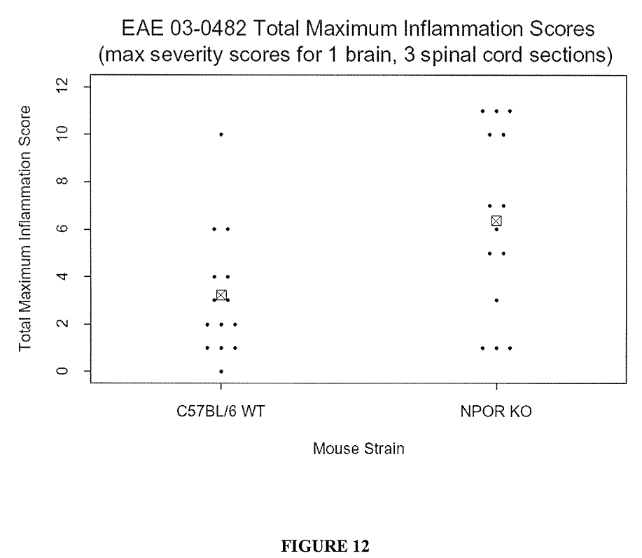

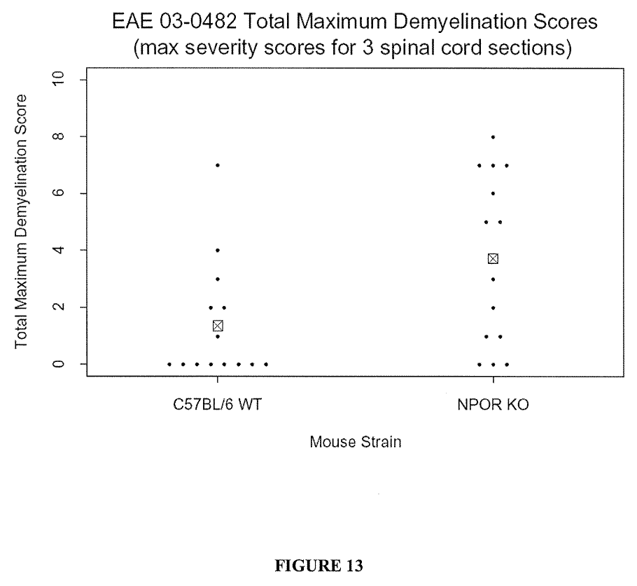

The clinical data was further supported by histological analysis as shown in FIGS. 10-13. TCCR−/− mice had higher inflammation and higher demyelination scores than WT mice, indicating that mice expressing TCCR (WT) were less susceptible to the CD4⁺ Th1 and CD8⁺ mediated disorder EAE than were mice lacking TCCR (−/−).

In sum, the data suggests a protective, dampening, or suppressive effect of TCCR activity against autoimmune disorders, such as EAE.

### Example 3

### Treatment of an Autoimmune Disorder with a TCCR Agonist

As described for Example 2, experimental allergic encephalomyelitis (EAE) is a CD4⁺ Th1 or CD8⁺ mediated autoimmune disorder of the CNS that serves as an animal model for multiple sclerosis (MS). To examine the effect of an administered TCCR agonist on the progression and course of an autoimmune disorder, a TCCR agonist such as IL-27 is administered in an experimental model system of MS, such as induced EAE. EAE is initiated in mice, for example, as described above for Example 2. Clinical progression of EAE is evaluated in mice expressing TCCR (TCCR +/+) and receiving a TCCR agonist, such as IL-27. Mice treated with a TCCR agonist such as IL-27 are expected to show reduced clinical symptoms or progression of disease, and/or to be less susceptible to autoimmune disorders than untreated TCCR +/+controls.

### Example 4

### Treatment of Arthritis in an Animal Model with a TCCR Agonist

The suppressive and/or protective effect of a TCCR agonist on autoimmune disorders can be tested in one of several available animal model systems. Collagen-induced arthritis in mice is one model for the autoimmune disorder, rheumatoid arthritis. This model is described, for example, in McIndoe et al., 1999, PNAS USA 96:2210-2214. Collagen-induced arthritis in mice shares many features with human rheumatoid arthritis, including lymphocytic infiltration and synovial membrane hypertrophy.

Clinical progression of collagen-induced arthritis is examined in mice, for example C57BL/6 mice or other suitable laboratory animals. Arthritis is induced in the test animals, for example, by the methods recited in McIndoe et al, supra, or other known methods. In general, the model animals are generated by injecting a type II collagen derived from a different animal species into the test animals, for example bovine type II collagen into mice. The collagen may be combined with an adjuvant, such as complete Fruend's adjuvant.

A TCCR agonist, such as the TCCR ligand IL-27, is administered to the test animals, for example, prior to, during, and/or post administration of the arthritis-inducing agent or prior to, during, and/or post onset of arthritic symptoms. Methods of administration and dosages can vary, and include for example, administration of a peptide ligand such as IL-27 in a carrier, for example, in one pre and/or post dose, in multiple doses per day, daily over a period of two or more pre and/or post doses, or other suitable dosages known to administer a peptide agent to the cells expressing the TCCR receptor. Alternatives include delivery of a peptide ligand such as IL-27 by expressing the peptide from a recombinant adenovirus, for example, expressing both subunits of IL-27, or a linked IL-27 cytokine.

Progression of clinical disease is monitored in test and control animals, for example, as described in McIndoe et al., supra. For example, physical and chemical characteristics of the disease are monitored and scored over a period of time. Animals may be analyzed for lymphocytic infiltration of major joints, synovial membrane hypertrophy, cytokine content in synovial fluid, and the like. These parameters are compared between test animals and controls. In keeping with the protective/suppressive effects of TCCR demonstrated in Examples 1 and 2, treatment of induced arthritis in animals with a TCCR agonist is expected to provide a suppressive and/or protective effect, demonstrated in less severe clinical symptoms, outcome, and/or physical or chemical characteristics as compared with untreated controls.

### Example 5

### Preparation of Monoclonal Antibodies to TCCR

Monoclonal antibodies to hTCCR were prepared using the extracellular domain of hTCCR. The immunogen was hTCCR (SEQ ID NO: 1) lacking the transmembrane portion (residues 517 to 538 of SEQ ID NO: 1) tagged with eight histidine residues added to the carboxy-terminus for purification purposes. The hTCCR(ECD)-(His)₈ peptide was purified through nickel NTA affinity chromatography.

The hTCCR(ECD)-(His)₈ peptide (1-2 micrograms) was combined with 25 microliters of MPL-TDM adjuvant (Ribi Immunochemical Research, Hamilton, Mont.) and injected into the footpads of wild-type balb/c mice (Charles River Laboratories, Wilmington, Mass.) twice weekly for a total of 12 injections.

On day 42 the mice were sacrificed and spleen cells were harvested. The spleen cells were fused (using 35% polyethylene glycol) to murine myeloma cells (P3X63AgU.1, available from ATCC, No. CRL 1597). The fusions generated hybridoma cells that were plated in 96 well tissue culture plates containing HAT (hypoxanthine, aminopterin, and thymidine) medium to inhibit proliferation of non-fused cells, myeloma hybrids, and spleen cell hybrids.

Hybridoma cells were then screened in an ELISA assay for antibody binding to TCCR. Hybridoma cultures identified having reactivity to TCCR included cultures: 2685, 2686, and 2688. The hybridoma culture 2686 (antibody 2686) was deposited with the American Type Culture Collection (ATCC), Manassas, Va., on Dec. 15, 2004, and has Accession Number ATCC ______.

### Example 6

### Monoclonal Ab 2686 Activates Human TCCR

Ba/F3 cells expressing recombinant TCCR were used to analyze the ability of anti-TCCR antibodies to activate TCCR. Ba/F3 cells are a murine IL-3 dependent cell line. Candidate agonists of TCCR can be evaluated by measuring proliferation of Ba/F3 cells expressing TCCR in response to the candidate agonist. Cell proliferation results in increased incorporation of [³H]-thymidine because of increased synthesis of polynucleotides. Cell proliferation is monitored, for example, by measuring [³H]-thymidine uptake. As shown below, monoclonal AB 2686 demonstrated TCCR agonist activity by inducing proliferation of Ba/F3 cells expressing TCCR.

Ba/F3 cells (Palacios et al., 1985, Cell, 41:727-734) are a murine hematopoietic factor-dependent cell line requiring IL-3 for both growth and survival. Ba/F3 cells were cultured in RPMI-1640 medium (GIBCO, Carlsbad, Calif.) supplemented with 10% fetal calf serum (GIBCO, Carlsbad, Calif.) and 100 pg/mL mouse IL-3 (R&D Systems, Minneapolis, Minn.).

A pMSCV vector (Clontech, Palo Alto, Calif.) with a neomycin resistance gene and containing either the polynucleotide sequence encoding human or murine TCCR was transfected into Ba/F3 cells by electroporation. Stable transfectants were treated with 1 mg/ml of G418 (Clontech, Palo Alto, Calif.) to select stable eukaryotic cell lines that have been transfected with vectors containing the gene for neomycin resistance. Cells were then treated with phyco-erythrin labeled monoclonal antibodies recognizing TCCR. Labeled clones expressing TCCR were selected by FACS.

TCCR expressing cells were washed with RPMI-1640 medium supplemented with 10% fetal calf serum without added IL-3. The cells were then plated in duplicate at 5×10³ cells per well in 100 μl of RPMI-1640 medium supplemented with 10% fetal calf serum. Purified recombinant murine IL-3 (positive control) or purified anti-TCCR(human) monoclonal antibodies: 2685-IgG2a, 2686-IgG1, 2688-IgG1, control isotype IgG2a (BD Pharmingen, San Diego, Calif.), or control isotype IgG1 (BD Pharmingen, San Diego, Calif.) were added at concentrations indicated below in Table 14 as a 4:1 dilution series.

TABLE 14

| Positive Control (ng/ml) | Idiotype Controls (ug/ml) | | Test Antibodies (ug/ml) | | |
|---|---|---|---|---|---|
| IL-3 | IgG1 | IgG2a | 2685 | 2686 | 2688 |
| 100 | 100 | 100 | 100 | 100 | 100 |
| 25 | 25 | 25 | 25 | 25 | 25 |
| 6.25 | 6.25 | 6.25 | 6.25 | 6.25 | 6.25 |
| 1.56 | 1.56 | 1.56 | 1.56 | 1.56 | 1.56 |
| 0.39 | 0.39 | 0.39 | 0.39 | 0.39 | 0.39 |
| 9.76 × 10⁻² | 9.76 × 10⁻² | 9.76 × 10⁻² | 9.76 × 10⁻² | 9.76 × 10⁻² | 9.76 × 10⁻² |
| 2.44 × 10⁻² | 2.44 × 10⁻² | 2.44 × 10⁻² | 2.44 × 10⁻² | 2.44 × 10⁻² | 2.44 × 10⁻² |
| 6.10 × 10⁻³ | 6.10 × 10⁻³ | 6.10 × 10⁻³ | 6.10 × 10⁻³ | 6.10 × 10⁻³ | 6.10 × 10⁻³ |
| 1.52 × 10⁻³ | 1.52 × 10⁻³ | 1.52 × 10⁻³ | 1.52 × 10⁻³ | 1.52 × 10⁻³ | 1.52 × 10⁻³ |
| 3.81 × 10⁻⁴ | 3.81 × 10⁻⁴ | 3.81 × 10⁻⁴ | 3.81 × 10⁻⁴ | 3.81 × 10⁻⁴ | 3.81 × 10⁻⁴ |
| 9.53 × 10⁻⁵ | 9.53 × 10⁻⁵ | 9.53 × 10⁻⁵ | 9.53 × 10⁻⁵ | 9.53 × 10⁻⁵ | 9.53 × 10⁻⁵ |

After 48 hours, 1 μCi of [³H]-thymidine (Amersham-Pharmacia, Piscataway, N.J.) was added to each well. After 6 additional hours, incorporation of the [³H]-thymidine into cells was measured in a β-counter (Packard Topcount, PerkinElmer Life and Analytical Sciences, Boston, Mass.).

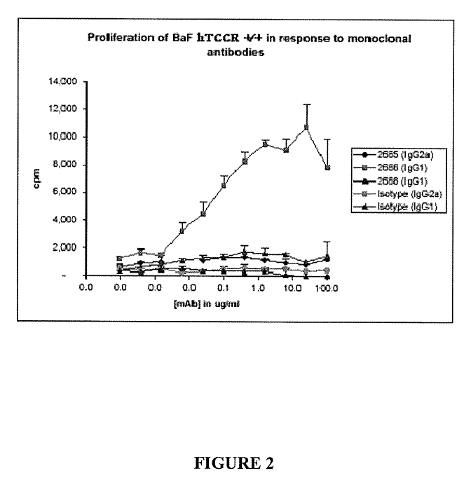

The results are shown in FIGS. 2-4. Proliferation of Ba/F3 cells expressing human TCCR in response to monoclonal antibodies 2685-IgG2a, 2686-IgG1, 2688-IgG1, and control isotype IgG2a and isotype IgG1 is shown in FIG. 2. Antibody 2686 induced significantly greater incorporation of [3H]-thymidine than any of the other antibodies tested, demonstrating that antibody 2686 is an effective agonist of human TCCR expressed in Ba/F3 cells.

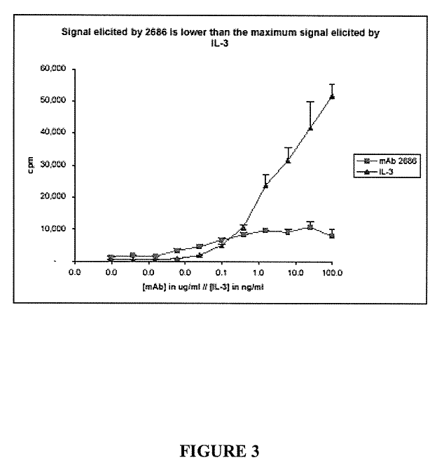

Proliferation of Ba/F3 cells expressing human TCCR in response to either murine IL-3 (positive control) or antibody 2686 is shown in FIG. 3. As shown, antibody 2686 was effective in generating a TCCR response in Ba/F3 cells expressing human TCCR, albeit less than that of the positive control IL-3.

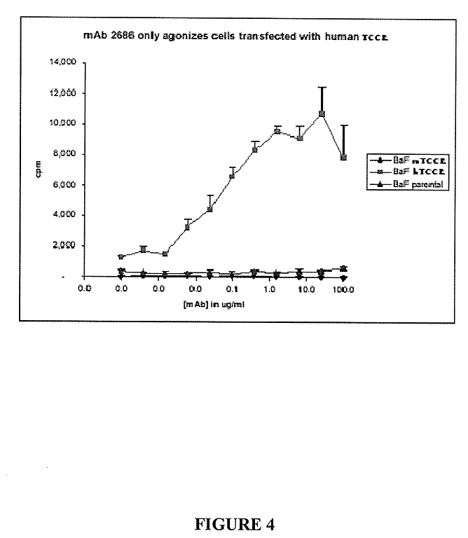

Ba/F3 cells expressing human TCCR incorporated significantly larger amounts of [3H]-thymidine in response to treatment with antibody 2686 than did the Ba/F3 cells expressing murine TCCR, as shown in FIG. 4. This data demonstrates that antibody 2686 is a specific agonist of human TCCR, and shows no cross-reactivity with murine TCCR.

These studies demonstrate that agonists of TCCR, such as the demonstrated agonist antibodies, can bind and stimulate the TCCR receptor to induce TCCR-mediated biological activity, here, proliferation of Ba/F3 cells. Accordingly, the data suggest TCCR agonists are useful to induce, directly or indirectly, TCCR-mediated activity in vivo.

### Example 7

### Identification of Other TCCR Agonists

To identify and confirm agents having TCCR agonistic activity, putative TCCR agonists, including fragments of IL-27 and variants of TCCR, are analyzed for binding to the TCCR receptor. TCCR binding can be analyzed in vitro or in vivo. For example, a potential agonist is administered to cells expressing TCCR, such as COS cells or Ba/F3 cells engineered to express recombinant TCCR, as described above for Example 3, and measuring cellular response to the potential agonist.

Receptor binding can also be analyzed by expressing a potential peptide agonist as a fusion protein, for example an immunoadhesin containing the Fc domain of human IgG. Receptor-ligand binding is detected, for example, by allowing interaction of the immunoadhesin with TCCR expressing cells. Bound immunoadhesin can be microscopically visualized, using fluorescent reagents that recognize the Fc fusion domain. Binding can be quantitated by analysis of fluorescence, or by other known methods.

Agonists of TCCR can be screened by analyzing the ability of the candidate agonist to stimulate a TCCR mediated activity such as expression of IL-10 or SOCS-3. For example, T-lymphocytes expressing TCCR can be contacted with a candidate agonist. Expression of IL-10 and/or SOCS-3 can be measured, for example, by ELISA, quantitative PCR, and the like methods. An increase in the expression of IL-10 and/or SOCS-3 relative to a control, for example, basal IL-10 and/or SOCS-3 levels, is correlated with TCCR stimulation, and indicative of a useful TCCR agonist.

### Example 8

### IL-27 Mediated Cell Proliferation and Induction and Suppression of Cytokines

The effect of IL-27 on cytokine induction in both wild-type (TCCR +/+) and knock-out (TCCR −/−) CD4⁺ cells was examined under neutral, Th1, or Th2 inducing conditions. The effect of IL-27 on cellular recall proliferation in both wild-type (TCCR +/+) and knock-out (TCCR −/−) CD4⁺ cells was examined under neutral, Th1, or Th2 inducing conditions.

On day 0, wild-type CD4^{|} or TCCR knock-out CD4^{|} cells were plated at 2×10⁵ cells per well in 24 well plates that had previously been coated with agonistic anti-CD3 monoclonal antibodies (145-2C11, BD Pharmingen, San Diego, Calif., 5 ug/ml in PBS o/n). Proliferation in individual wells was then induced under neutral, Th1 biasing, or Th2 biasing conditions. Neutral conditions were created by addition of IL-2 (R&D Systems, Minneapolis, Minn.), anti-IL-12 antibodies (BD Pharmingen, San Diego, Calif.), anti-IFN-γ antibodies (BD Pharmingen, San Diego, Calif.), anti-IL-4 antibodies (BD Pharmingen, San Diego, Calif.), and CD-28 (BD Pharmingen, San Diego, Calif.). Th1 biasing conditions were created by addition of IL-2, IL-12 (R&D Systems, Minneapolis, Minn.), anti-IL-4 antibodies, and CD-28. Th2 biasing conditions were created by addition of IL-2, IL-4 (R&D Systems, Minneapolis, Minn.), anti-IL-12 antibodies, anti-IFN-γ antibodies, and CD28. Treatment in these individual wells is shown below in Table 15.

TABLE 15

| | |
|---|---|
| Neutral | 1 ml of media (IMDM w/ 10% FBS (HyClone, Logan, UT)) |
| w/o IL-27 | containing factors at the following final concentrations |
| | 2 × 10⁻⁴ mg/ml IL-2 |
| | 5 × 10⁻³ mg/ml anti-IL-12 antibody |
| | 5 × 10⁻³ mg/ml anti-IFN-γ antibody |
| | 5 × 10⁻³ mg/ml anti-IL-4 antibody |
| | 1 × 10⁻³ mg/ml CD28 |
| Neutral | 1 ml of media (IMDM w/ 10% FBS) containing factors at the |
| w/ IL-27 | following final concentrations |
| | 2 × 10⁻⁴ mg/ml IL-2 |
| | 5 × 10⁻³ mg/ml anti-IL-12 antibody |
| | 5 × 10⁻³ mg/ml anti-IFN-γ antibody |
| | 5 × 10⁻³ mg/ml anti-IL-4 antibody |
| | 1 × 10⁻³ mg/ml CD28 |
| | 2 × 10⁻⁴ mg/ml IL-27 |
| TH1 | 1 ml of media (IMDM w/ 10% FBS) containing factors at the |
| w/o IL-27 | following final concentrations |
| | 2 × 10⁻⁴ mg/ml IL-2 |
| | 4 × 10⁻⁶ mg/ml IL-12 |
| | 5 × 10⁻³ mg/ml anti-IL-4 antibody |
| | 1 × 10⁻³ mg/ml CD28 |
| TH1 | 1 ml of media (IMDM w/ 10% FBS) containing factors at the |
| w/ IL-27 | following final concentrations |
| | 2 × 10⁻⁴ mg/ml IL-2 |
| | 5 × 10⁻³ mg/ml anti-IL-12 antibody |
| | 5 × 10⁻³ mg/ml anti-IL-4 antibody |
| | 1 × 10⁻³ mg/ml CD28 |
| | 2 × 10⁻⁴ mg/ml IL-27 |
| TH2 | 1 ml of media (IMDM w/ 10% FBS) containing factors at the |
| w/o IL-27 | following final concentrations |
| | 2 × 10⁻⁴ mg/ml IL-2 |
| | 4 × 10⁻⁶ mg/ml IL-4 |
| | 5 × 10⁻³ mg/ml anti-IL-12 antibody |
| | 5 × 10⁻³ mg/ml anti-IFN-γ antibody |
| | 1 × 10⁻³ mg/ml CD28 |
| TH2 | 1 ml of media (IMDM w/ 10% FBS) containing factors at the |
| w/ IL-27 | following final concentrations |
| | 2 × 10⁻⁴ mg/ml IL-2 |
| | 4 × 10⁻⁶ mg/ml IL-4 |
| | 5 × 10⁻³ mg/ml anti-IL-12 antibody |
| | 5 × 10⁻³ mg/ml anti-IFN-γ antibody |
| | 1 × 10⁻³ mg/ml CD28 |
| | 2 × 10⁻⁴ mg/ml IL-27 |

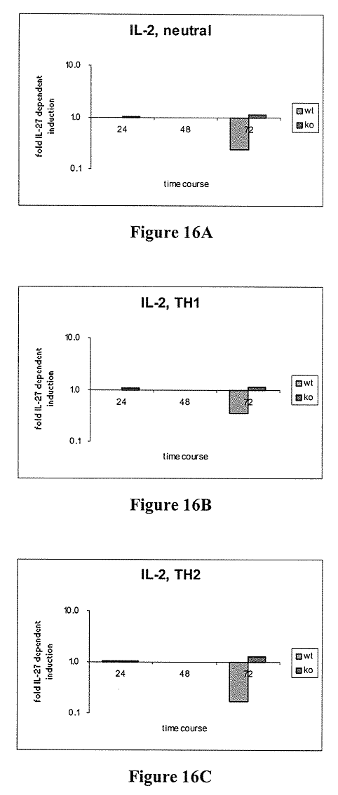

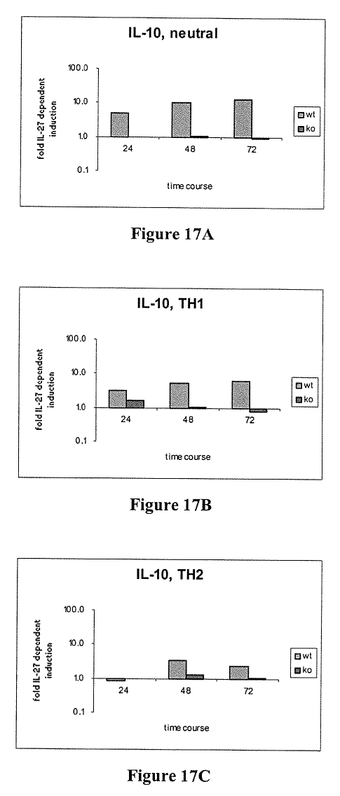

Cells were cultured at 37 degrees Celsius. Samples of the supernatant were taken at 24 hours, 48 hours, and/or 72 hours. ELISA was performed on the supernatant samples with probes for TNF-α, IL-5, IL-2, IFN-γ, IL-10, IL-6, IL-4, GM-CSF (kits purchased from BD Pharmingen, San Diego, Calif.). Table 16 below shows the ELISA data as fold IL-27 dependent induction. FIGS. 16A-C show IL-27 dependent induction of IL-2 under neutral (16A), Th1 biasing (16B), and Th2 biasing conditions (16C). FIGS. 17A-C show IL-27 dependent induction of IL-10 under neutral (17A), Th1 biasing (17B), and Th2 biasing conditions (17C).

The data show induction of TNF-α, IFN-γ, and IL-4 in response to IL-27. The data also show suppression of IL-2, IL-6, and GM-CSF in response to IL-27. The data show that IL-10 is induced by IL-27 under neutral, Th1 biasing, and Th2 biasing conditions. As stated above, IL-10 plays a major role in limiting and terminating inflammatory responses. As IL-10 is induced by IL-27, the data suggest that IL-27 can be used to treat immune-mediated diseases.

TABLE 16
Cytokine Induction by IL-27

| | | IL-27 (200 ng/ml) | | | | | | No IL-27 | | |
|---|---|---|---|---|---|---|---|---|---|---|
| | | TCCR wt | | | TCCR ko | | | TCCR wt/TCCR ko | | |
| | Time | N | TH1 | TH2 | N | TH1 | TH2 | N | TH1 | TH2 |
| TNFα | 24 hrs | | 0.5 | 0.7 | | 8.1 | | | 0.1 | 0.8 |
| | 48 hrs | | | | | | | | | |
| | 72 hrs | 5.3 | 3.9 | 1.9 | 1 | 0.9 | | 21 | 1.6 | 1.1 |
| IL-5 | 24 hrs | 2.9 | | | 1.5 | 1 | 1 | 9.8 | | |
| | 48 hrs | | | | | | | | | |
| | 72 hrs | 0.3 | 0.6 | 0.4 | 1 | 1.1 | 1 | 25 | 3.6 | 1.5 |
| IL-2 | 24 hrs | 1 | 1 | 1 | 1 | 1.1 | 1.1 | 1.1 | 1 | 0.9 |
| | 48 hrs | | | | | | | | | |
| | 72 hrs | 0.2 | 0.4 | 0.2 | 1.1 | 1.1 | 1.3 | 0.8 | 0.7 | 0.4 |
| IFNγ | 24 hrs | | 4.5 | | | 1 | | | 7.1 | |
| | 48 hrs | | 1.6 | | | 1.3 | | | 1.1 | |
| | 72 hrs | | 1.3 | | | 0.8 | | | 1.1 | |
| IL-10 | 24 hrs | 5.1 | 3.2 | 0.8 | | 1.7 | | | 1.1 | |
| | 48 hrs | 10.4 | 5.3 | 3.4 | 1.1 | 1.1 | 1.3 | 1.1 | 1.3 | 1.1 |
| | 72 hrs | 13.1 | 5.9 | 23 | 0.9 | 0.8 | 1 | 1.1 | 1.5 | 2.3 |
| IL-6 | 24 hrs | | | | | 0.1 | | | | |
| | 48 hrs | | | | | | | | | |
| | 72 hrs | 0.3 | 0.5 | 0.3 | 0.9 | 1 | 0.9 | 0.9 | 1.3 | 1.2 |
| IL-4 | 24 hrs | 4.5 | | 1 | 2.7 | | 1 | 35.8 | | 1 |
| | 48 hrs | | | 2.1 | | | 1 | | | 0.7 |
| | 72 hrs | | | 4 | | | 1 | | | 0.9 |
| GM-CSF | 24 hrs | 0.7 | 0.6 | 2.4 | 0.9 | 4.4 | 0.5 | 27 | 0.9 | 1.6 |
| | 48 hrs | 0.1 | 0.2 | 0.2 | 0.8 | 1.1 | 1 | 22 | 1.3 | 0.8 |
| | 72 hrs | 0.1 | 0.1 | 0.1 | 0.8 | 0.9 | 1 | 1.2 | 1.4 | 1.1 |

*data shown as fold induction by IL-27

RNA was extracted from the cell samples taken at 24 hours, 48 hours, and/or 72 hours and then quantitative PCR (TAQMAN®) was performed with probes specific for SOCS-1, SOCS-3, PIAS-1, and PIAS-3 as shown in Table 17 below.

TABLE 17

| | |
|---|---|
| Probes and Primers | Sequence |

| | |
|---|---|
| mSOCS1.DNA240484 | TGGTTGTAGCAGCTTGTGTCT |
| forward | (SEQ ID NO: 9) |
| mSOCS1.DNA240484 | GTGCAAAGATACTGGGAATATGTAA |
| reverse | (SEQ ID NO: 10) |
| mSOCS1.DNA240484 | CCAGGACCTGAATTCCACTCCTACCTC |
| probe | (SEQ ID NO: 11) |
| mSOCS3.AK047165 | TCCTGAGTTAACACTGGGAAGA |
| forward | (SEQ ID NO: 12) |
| mSOCS3.AK047165 | GGAGGCTCTCGGACCTACT |
| reverse | (SEQ ID NO: 13) |
| mSOCS3.AK047165 | ATTGGCCAGTCCTAGTCATCTCTCGGT |
| probe | (SEQ ID NO: 14) |
| mPIAS1.AK075708 | GATGGCAACTGATGGAGGAT |
| forward | (SEQ ID NO: 15) |
| mPIAS1.AK075708 | AGTGCAGGAGCTGGTGATG |
| reverse | (SEQ ID NO: 16) |
| mPIAS1.AK075708 | TGTGCCCTGGCTCTCTGCAGTTAC |
| probe | (SEQ ID NO: 17) |
| mPIAS3.BC051252 | ATCCCTCAGGGGTCATTG |
| forward | (SEQ ID NO: 18) |
| mPIAS3.BC051252 | GGCCAAAAGCAGGTATCC |
| reverse | (SEQ ID NO: 19) |
| mPIAS3.BC051252 | CAAAGGCCAGGCCAGAGCTTCA |
| probe | (SEQ ID NO: 20) |

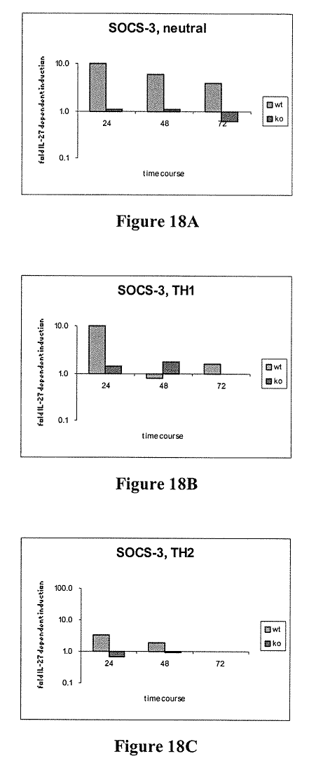

Table 18 below shows the quantitative PCR data as fold IL-27 dependent induction. FIGS. 18A-C show IL-27 dependent induction of SOCS-3 under neutral (18A), Th1 biasing (18B), and Th2 biasing conditions (18C).

The data show that SOCS-3 is induced by IL-27 under neutral, Th1 biasing, and Th2 biasing conditions. As stated above, SOCS-3 is known to suppress cytokine signaling, and has been reported to be the mediator of the anti-inflammatory effect of some agents. As SOCS-3 is induced by IL-27, the data suggest that IL-27 can be used to treat immune-mediated diseases.

TABLE 18

| | | IL-27 (200 ng/ml) | | | | | | No IL-27 | | |
|---|---|---|---|---|---|---|---|---|---|---|
| | | TCCR wt | | | TCCR ko | | | TCCR wt/TCCR ko | | |
| | Time | N | TH1 | TH2 | N | TH1 | TH2 | N | TH1 | TH2 |
| SOCS1 | 24 hrs | 3.1 | 2.8 | 2.0 | 0.7 | 2.2 | 0.6 | 1.2 | 1.0 | 2.5 |
| | 48 hrs | 1.0 | 1.0 | 2.0 | 0.8 | 0.9 | 0.8 | 0.8 | 1.0 | 1.2 |
| | 72 hrs | 1.1 | 0.6 | 1.9 | 0.7 | 1.2 | 1.2 | 1.6 | 0.6 | 1.5 |
| SOCS3 | 24 hrs | 10.0 | 9.8 | 3.3 | 1.1 | 1.4 | 0.7 | 3.2 | 3.8 | 6.0 |
| | 48 hrs | 5.8 | 0.8 | 1.9 | 1.1 | 1.7 | 0.9 | 2.8 | 1.0 | 1.9 |
| | 72 hrs | 3.9 | 1.6 | 1.0 | 0.6 | 1.0 | 1.0 | 3.0 | 1.4 | 2.6 |
| PIAS1 | 24 hrs | 0.7 | 1.6 | 1.0 | 1.4 | 1.2 | 0.6 | 0.8 | 1.9 | 3.2 |
| | 48 hrs | 1.0 | 1.2 | 1.9 | 1.5 | 1.1 | 1.1 | 1.3 | 1.4 | 1.5 |
| | 72 hrs | 1.8 | 0.8 | 1.2 | 0.7 | 1.3 | 1.0 | 2.0 | 1.0 | 2.0 |
| PIAS3 | 24 hrs | 1.3 | 1.4 | 0.3 | 0.8 | 1.2 | 0.6 | 1.9 | 1.6 | 1.0 |
| | 48 hrs | 0.8 | 0.6 | 1.7 | 0.5 | 1.3 | 1.3 | 1.3 | 1.0 | 0.8 |
| | 72 hrs | 0.9 | 0.6 | 0.9 | 0.8 | 0.7 | 0.9 | 1.0 | 1.2 | 0.7 |

Samples of the cells above that were treated under neutral conditions were taken at 72 hours and RNA was extracted. The RNA was then analyzed for induced expression using GENECHIP® (Affymetrix, Santa Clara, Calif.). Table 19 below shows the GENECHIP® data as fold induction (repression) over untreated controls for selected genes.

The data here again show that IL-10 is induced by IL-27 under neutral conditions. As stated above, IL-10 plays a major role in limiting and terminating inflammatory responses. As IL-10 is induced by IL-27, the data suggest that IL-27 can be used to treat immune-mediated diseases.

TABLE 19

Fold induction

| Gene | wild-type CD4+ | knock-out CD4+ |
|---|---|---|
| cathepsin W | 9.91 | 0.99 |
| interleukin 10 | 6.14 | 1.00 |
| TGF beta 3 | 5.32 | 1.22 |
| lymphocyte antigen 6 | 4.07 | 1.07 |
| complex, locus C | | |
| interleukin 2 | 0.02 | 1.48 |
| CD80 antigen | 0.10 | 0.82 |
| interleukin 13 | 0.12 | 0.79 |
| CD83 antigen | 0.17 | 0.81 |

On day 3, cells were expanded in the presence of IL-2 and presence or absence of IL-27. Specifically, those cells from the 24 well plates previously exposed to IL-27 were taken out in 1 ml of media and then deposited into 6 well plates along with 3 ml of medium containing 2×10⁻⁴ mg/ml IL-27 and 1×10⁻⁵ mg/ml IL-2. Those cells not previously exposed to IL-27 were taken out in 1 ml of media and then deposited into 6 well plates along with 3 ml of a medium containing 1×10⁻⁵ mg/ml IL-2.

On day 5, 4 ml of media (IMDM (Invitrogen, Carlsbad, Calif.) w/10% HyClone serum) having a concentration of 2×10⁻⁴ mg/ml IL-27 and 1×10⁻⁵ mg/ml IL-2 was added to those wells containing cells previously exposed to IL-27. 4 ml of media having a concentration of 1×10⁻⁵ mg/ml IL-2 was added to those wells containing cells not previously exposed to IL-27.

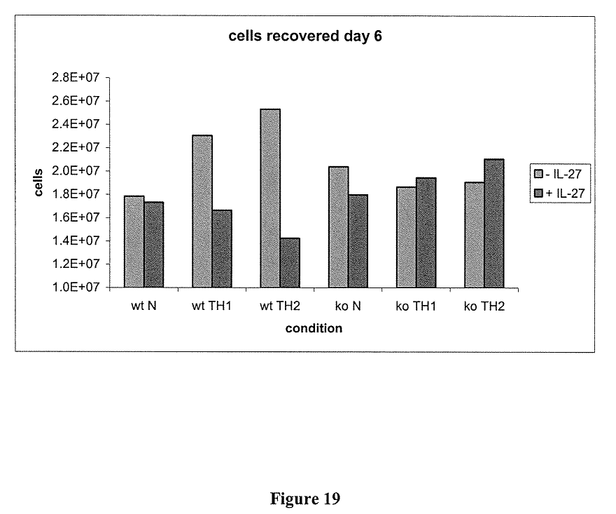

On day 6, the cells were centrifuged, and then the pellet re-suspended in media (same media as above) and counted. The cell counts from the various wells are shown below in Table 20 and reflected in FIG. 19.

The data show that IL-27 added during Th1 or Th2 biasing conditions reduces proliferation of CD4^{|} cells and suggests that IL-27 is useful to treat disease characterized by proliferation of CD4⁺ cells including autoimmune diseases such as multiple sclerosis and rheumatoid arthritis.

TABLE 20

| | | | Cell Count (Day 6) |
|---|---|---|---|
| IL-27 200 ng/ml | | N | 1.73E+07 |
| | TCCR wt | TH1 | 1.67E+07 |
| | | TH2 | 1.43E+07 |
| | | N | 1.80E+07 |
| | TCCR ko | TH1 | 1.95E+07 |
| | | TH2 | 2.11E+07 |
| No IL-27 | | N | 1.80E+07 |
| | TCCR wt | TH1 | 2.31E+07 |
| | | TH2 | 2.53E+07 |
| | | N | 2.04E+07 |
| | TCCR ko | TH1 | 1.87E+07 |
| | | TH2 | 1.91E+07 |

### Example 9

### IL-27 Suppression of IL-6 Induced Proliferation

The effect of IL-27 on IL-6 induced proliferation of wild-type (TCCR +/+) and knock-out (TCCR −/−) CD4⁺ cells was examined in the presence and absence of anti-IL-2 antibodies (BD Pharmingen, San Diego, Calif.).

Mixed splenocytes (4×10⁵) from wild-type mice were placed into wells on a 96-well plate. Mixed splenocytes (4×10⁵) from knock-out mice were placed into separate wells on the plate. All wells were coated with 100 μl of 2 ug/ml anti-CD3 in PBS o/n. Wells were treated in accord with the experimental groups shown below in Table 21.

TABLE 21

| | |
|---|---|
| Group 1 | No addition |
| Group 2 | 5 × 10⁻⁴ mg/ml IL-27 (Genentech, South San Francisco, |
| | CA) |
| Group 3 | 5 × 10⁻⁵ mg/ml IL-6 (R&D Systems, Minneapolis, MN) |
| Group 4 | 5 × 10⁻⁴ mg/ml of IL-27 and 5 × 10⁻⁵ mg/ml of IL-6 |
| Group 5 | 0.01 mg/ml anti-IL-2 antibodies |
| Group 6 | 0.01 mg/ml anti-IL-2 antibodies |
| | 5 × 10⁻⁴ mg/ml IL-27 |
| Group 7 | 0.01 mg/ml anti-IL-2 antibodies |
| | 5 × 10⁻⁵ mg/ml IL-6 |
| Group 8 | 0.01 mg/ml anti-IL-2 antibodies |
| | 5 × 10⁻⁴ mg/ml IL-27 and 5 × 10⁻⁵ mg/ml IL-6 |

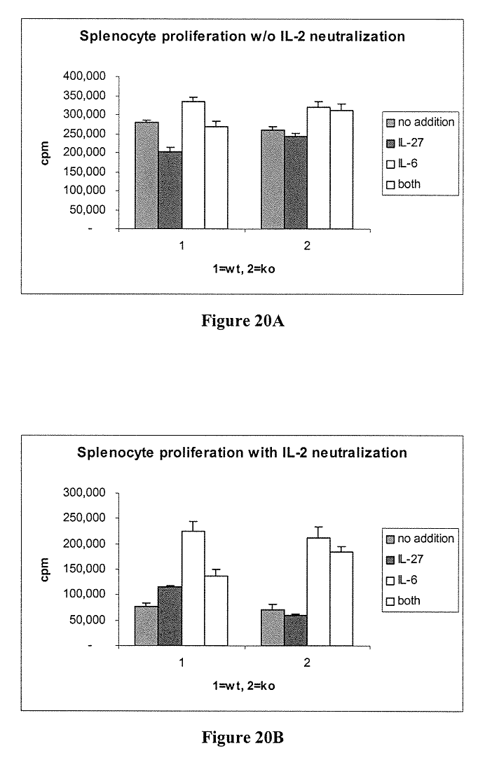

Cells were cultured at 37° C. After 48 hours, [³H]-thymidine was added for another night and proliferation was measured by [³H]-thymidine incorporation. The average CPM for each group is shown below in Table 22. Proliferation without IL-2 neutralization is shown in FIG. 20A. Proliferation with IL-2 neutralization is shown in FIG. 20B.

TABLE 22

| | | no addition | + IL-27 | + IL-6 | + IL-27 + IL-6 |
|---|---|---|---|---|---|
| wt spl | − anti IL-2 | 281302 | 202783 | 333361 | 267974 |
| | + anti IL-2 | 76741 | 116563 | 224512 | 136787 |
| ko spl | − anti IL-2 | 259217 | 243936 | 320718 | 312365 |
| | + anti IL-2 | 69672 | 59609 | 211305 | 184034 |

The data show that IL-27 represses proliferation stimulated by anti-CD3 antibodies and enhanced by IL-6, regardless of whether anti-IL-2 antibodies are present. When no anti-IL-2 antibodies are present, IL-27 represses proliferation stimulated by anti-CD3 antibodies. Anti-IL-2 antibodies reduce proliferation stimulated by anti-CD3 antibodies. However, addition of IL-27 partially mitigates this effect.

### Example 10

### IL-27 Receptor (TCCR) Deficient Mice are EAE Hypersensitive

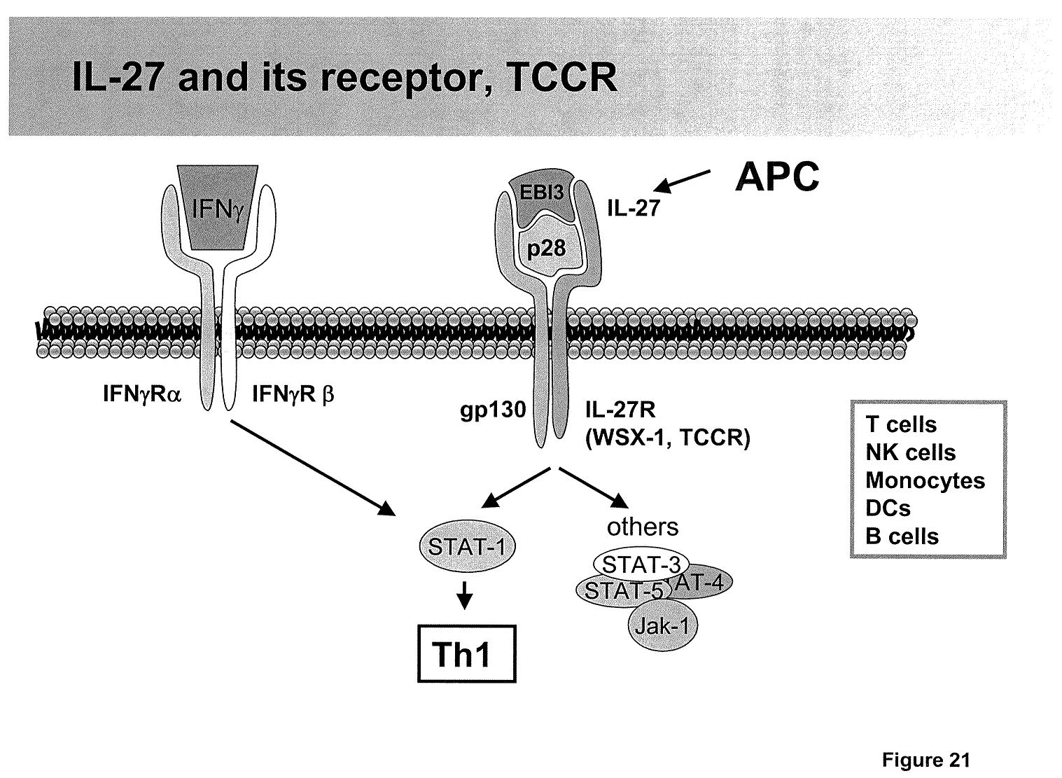

IL-27 is a ligand produced by activated antigen presenting cells (APC). IL-27 signals through a heterodimeric receptor consisting of a specific subunit, IL-27, and gp130 that is shared by a number of other receptors, including IL-6R. As discussed herein, IL-27 activates signals through various STATs and Jak-1, but the predominant signaling event appears to be activation of STAT-1. Through activation of STAT-1 and downstream induction of the TH-1 specific transcription factor T-bet, expression of the IL-12RB2 chain and IFN-gamma is promoted. The IL-27 ligand and receptor are shown diagrammatically in FIG. 21.

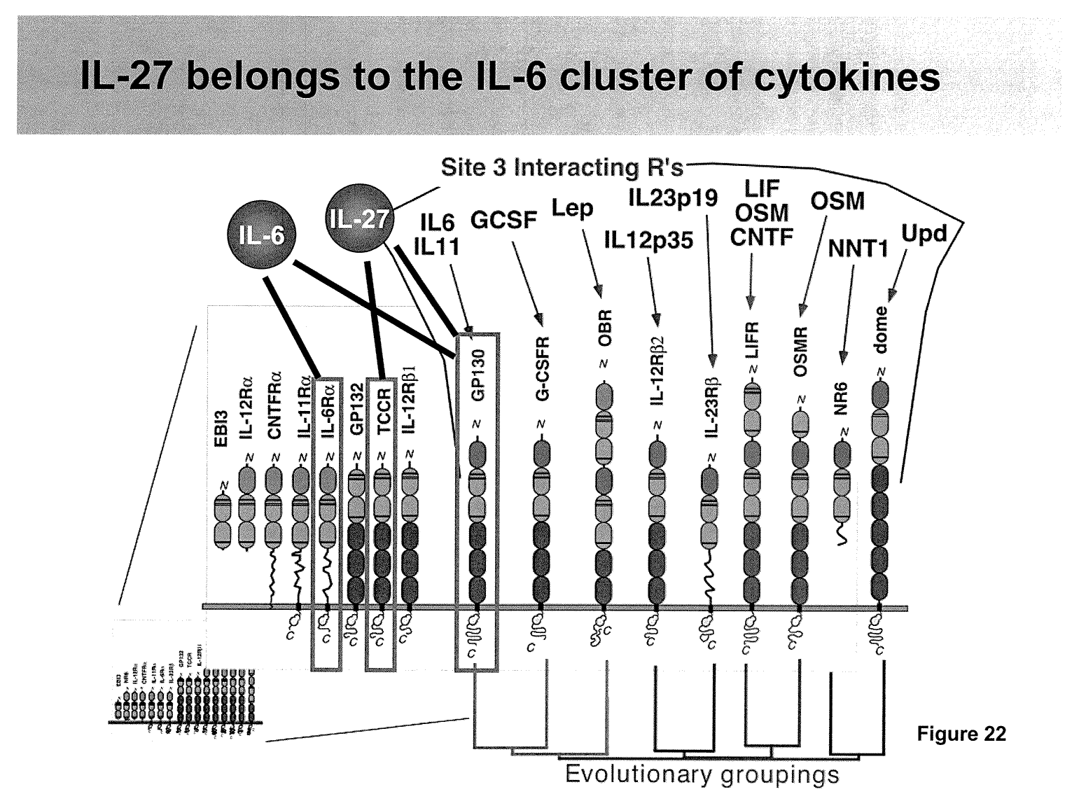

IL-27 is a member of the IL-12 family, and belongs to the IL-6 cluster of cytokines. See FIG. 22. The two components of IL-27, EBI3 and p28 share close homology to IL-12 subunits. Both subunits of the IL-27 receptor (IL-27R), also termed TCCR, are coordinately expressed on a variety of leukocytes. The highest expression appears to be on T cells and NK cells.

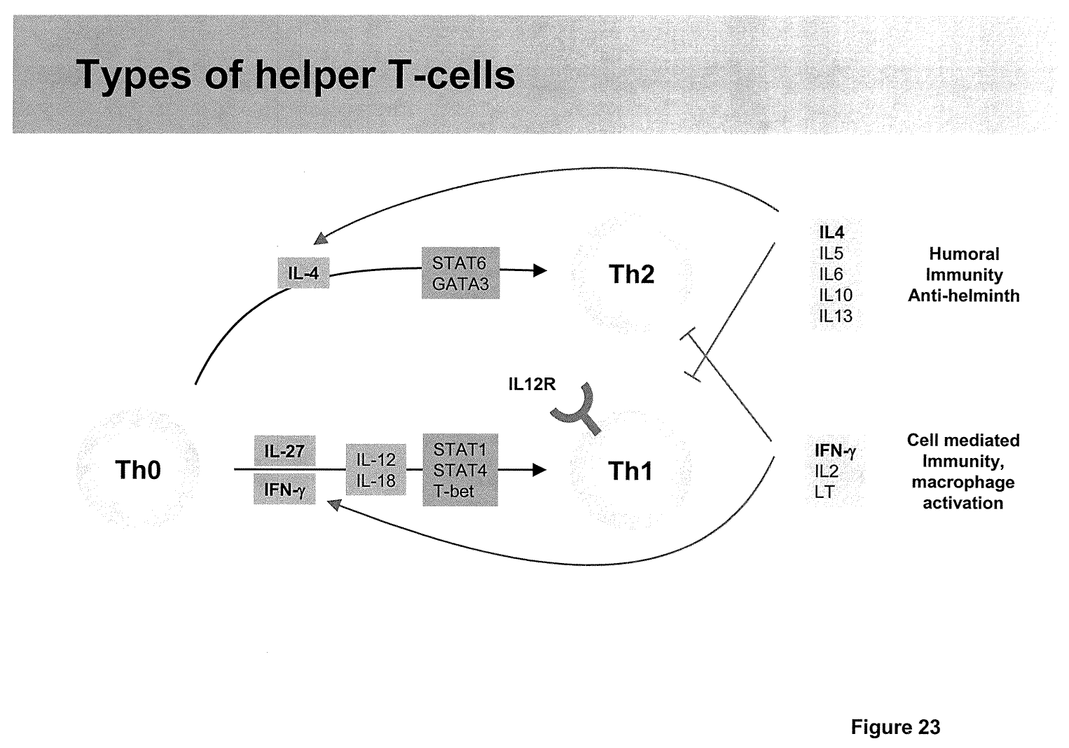

Naive, undifferentiated T cells (Th-0) respond to different signals that induce differentiation of naive Th-0 cells into mature T-helper cells. Generally, two types of T-helper cells are known, Th-1 and Th-2 cells. As diagramed in FIG. 23, stimulation of Th-0 cells by IL-4 leads to the development of Th-2 cells producing IL-4, IL-5, IL-6, IL-10, and IL-13. Th-2 cell and cytokine products impact humoral immunity and anti-helminth responses. Stimulation of Th-0 cells by IL-27 and/or IFN-gamma induces a state of IL-12 responsiveness in T-cells, so that they can differentiate into mature TH-1 cells under the control of IL-12, and produce IFN-gamma, IL-2, and Lymphotoxin (LT). Th-1 cells and their cytokine products are involved in cell-mediated immunity and macrophage activation.

To further our understanding of the role of IL-27 in the differentiation of Th-0 cells into Th-1 and Th-2 helper cells, IL-27R deficient mice (TCCR Knockout) were produced as described in the Examples above. A potential role for IL-27 during autoimmune disease was examined using experimental autoimmune encephalitis (EAE), a mouse model for Multiple Sclerosis. EAE is T cell mediated, since transfer of only CD4+ T cells from mice with EAE can cause EAE in naive recipient mice.

To induce experimental EAE, mice were immunized with myelin oligodendrocyte glycoprotein (MOG) 35-55 peptide in complete Freund's adjuvant. Wild type (WT) and IL-27 receptor (TCCR) knockout mice were immunized with MOG and examined for evidence of EAE as described in the Examples above. Clinical EAE score was evaluated over 25 days-post treatment.

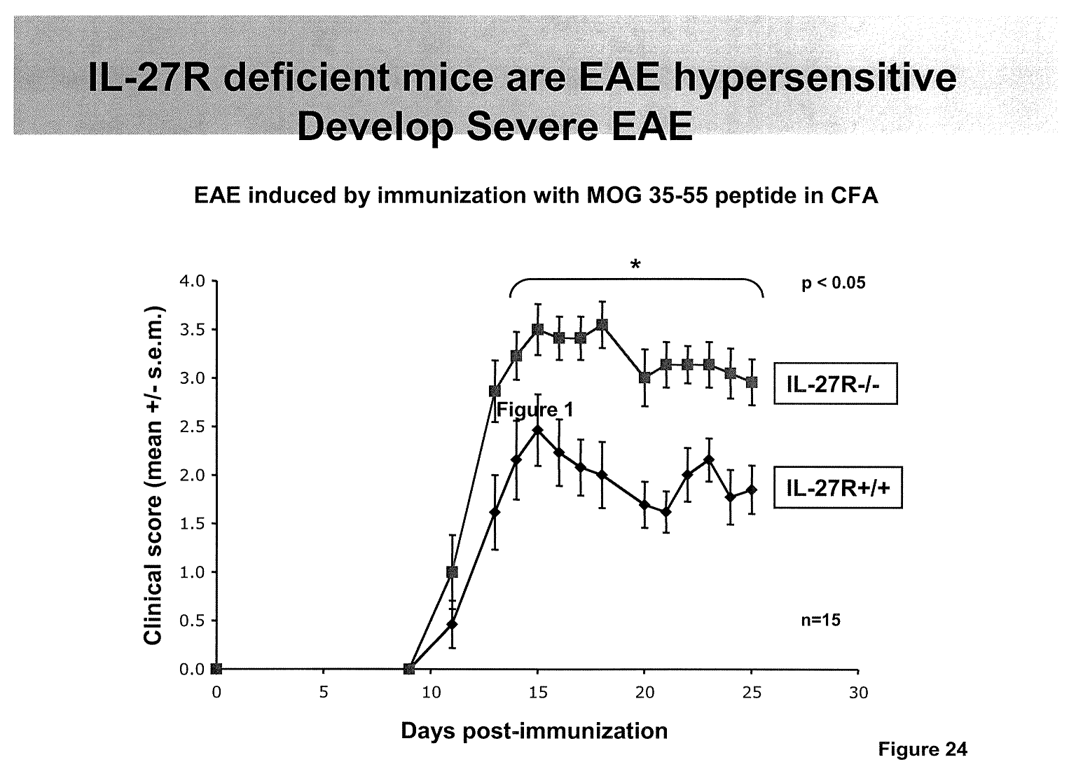

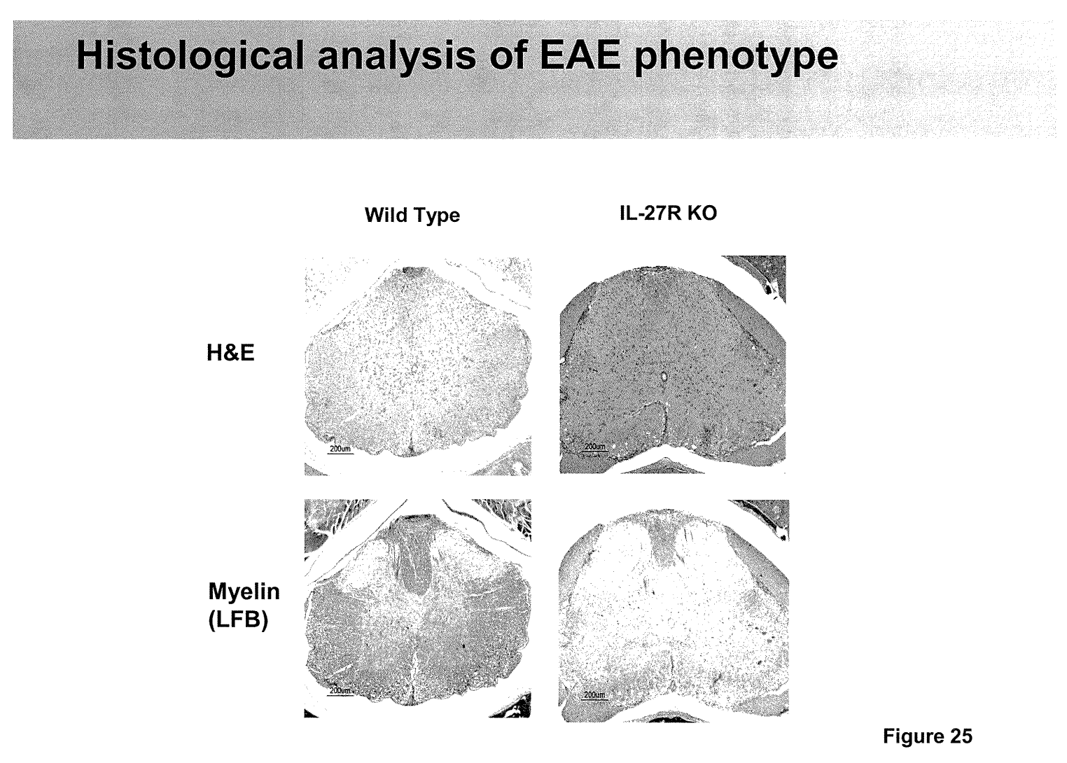

Data shown in FIG. 24 demonstrate that instead of the hypothesized reduction in EAE disease caused by removing the IL-27 stimulation, EAE was exacerbated in IL-27R deficient mice. The mice appeared to be EAE hypersensitive and developed severe EAE disease. Histological analysis of spinal cord tissue taken from receptor deficient mice expressing the EAE phenotype is shown in FIG. 25, and demonstrates enhanced inflammation and de-myelination in the IL-27 receptor knockout mice with EAE.

Further to this discovery, stimulation of IL-27 receptor deficient mice with a variety of pathogens, as well as induced asthma and hepatitis models, resulted in exacerbation of both Th-1 and Th-2 mediated responses. These data indicate that IL-27 has an important immunosuppressive function.

| Disease Model | IL-27 deficient mice | Reference |
|---|---|---|
| M. tuberculosis | Exacerbated Th1 response | Pearl et al., 2004, Immunol., 173(12): 7490-6.; |
| | | Holscher et al., 2003, J Immunol. 2005; 174: 3534-44 |
| T. gondii | Exacerbated Th1 response | Villarino et al., 2003, Immunity, 9: 645-55 |
| T. muris | Exacerbated Th2 response | Artis et al., 2004, J Immunol. 173: 5626-34 |
| Allergic Asthma | Exacerbated Th2 response | Miyazaki et al., 2004, J Immunol. 175: 2401-7 |
| ConA induced | Hyper inflammatory | Yamanakada et al., 2004, J Immunol. 172(6): 3590-6 |
| hepatitis | response | |

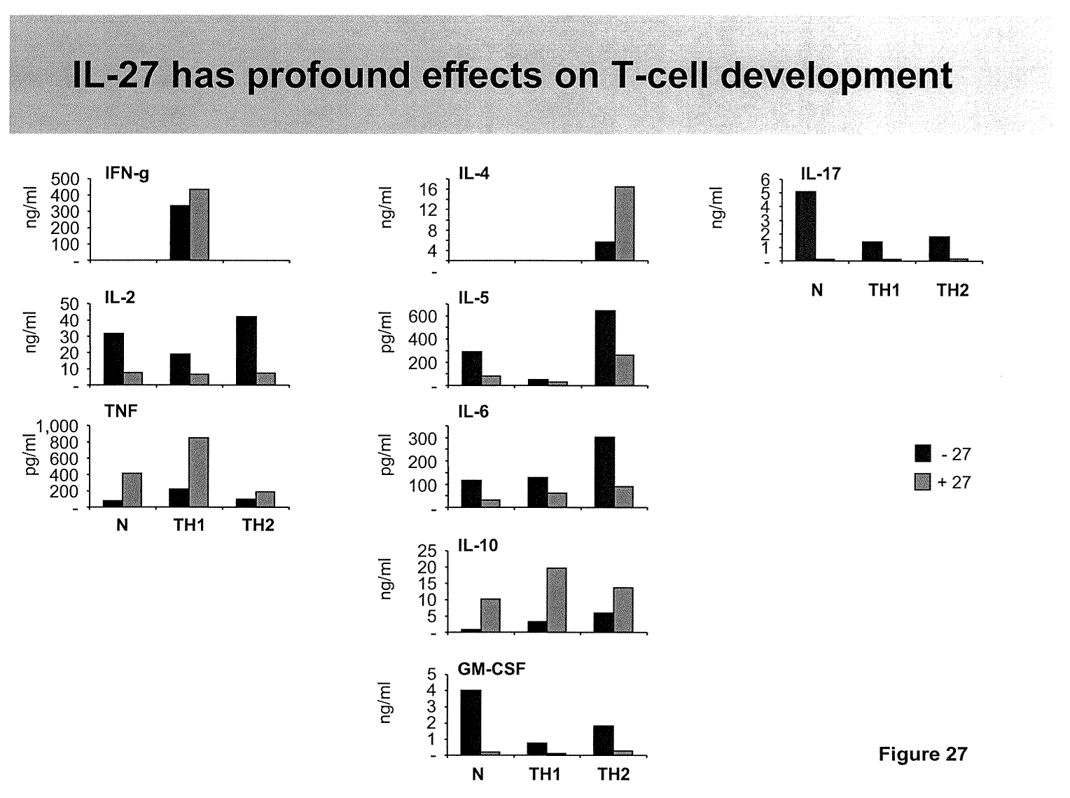

To further study IL-27 and its possible role in differentiation of T-cells, naïve CD4+ cells were MACS-purified and treated with anti-CD3+ antibody with or without added IL-27, according to the procedure diagrammed in FIG. 27. The stimulation of T cells with IL-27 was done under conditions that promote T cell polarization to Th-0, Th-1, or Th-2.

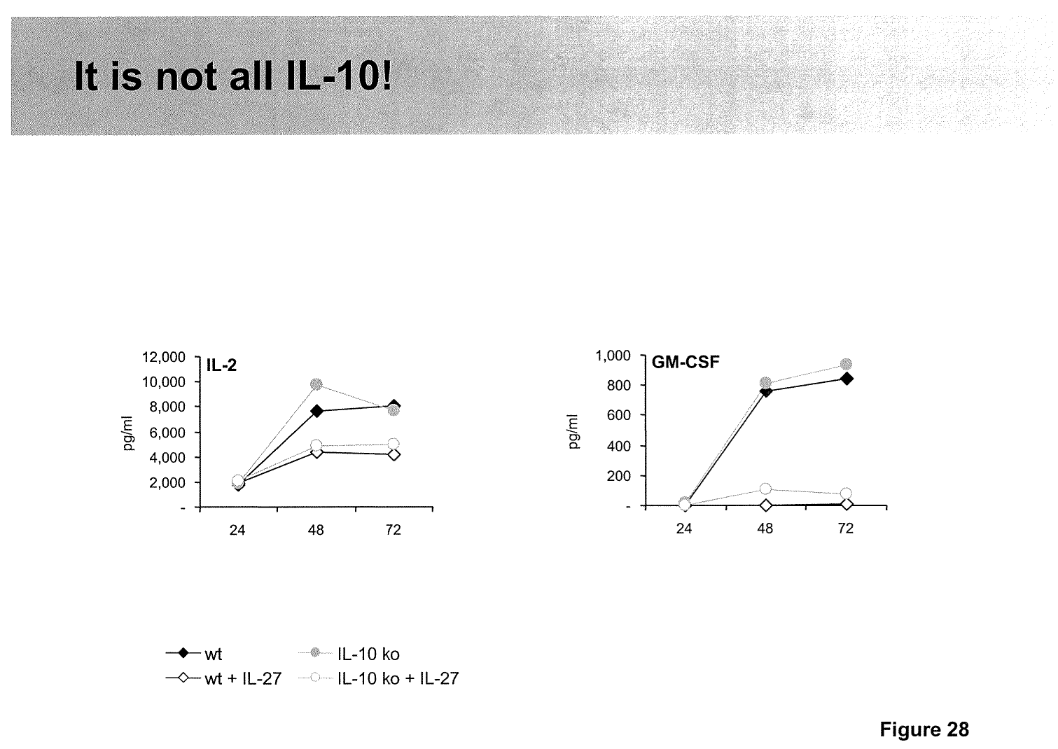

Briefly, 24 well dishes were coated overnight with 5 μg/ml anti-CD3 (BD Pharmingen). A volume of 1.8×10⁶ CD4+ T-cells were seeded per well in the presence of IL-2 (10 ng/ml) and anti-CD28 (1 μg/ml). For differentiation, the following cytokines and antibodies were added: TH-0 (anti-IL-12, anti-IFN-gamma, anti-IL-4 at 5 μg/ml each), TH-1 (IL-12 at 3.5 ng/ml, anti-IL-4 at 5 μg/ml), TH-2 (IL-4 at 3.5 ng/ml anti-IFNg and anti-IL-12 at 5 μg/ml). IL-27 was added to some cultures at a concentration of 200 ng/ml. After 72 hours, supernatants as well as RNA were isolated and analyzed for production of specific cytokines by Chip, RT-PCT, and/or ELISA analysis. The resultant data are shown in FIG. 28, and demonstrate that IL-27 had a profound effect on T-cell development.

IL-27 had a profound effect on most cytokines examined, and this effect was generally independent of the condition under which cells had been differentiated. IL-27 induced TNFα and IL-10, as well as IL-4 under Th-2 inducing conditions. At the same time, production of IL-2, IL-5, IL-6, GM-CSF, and IL-17 were profoundly suppressed by IL-27.

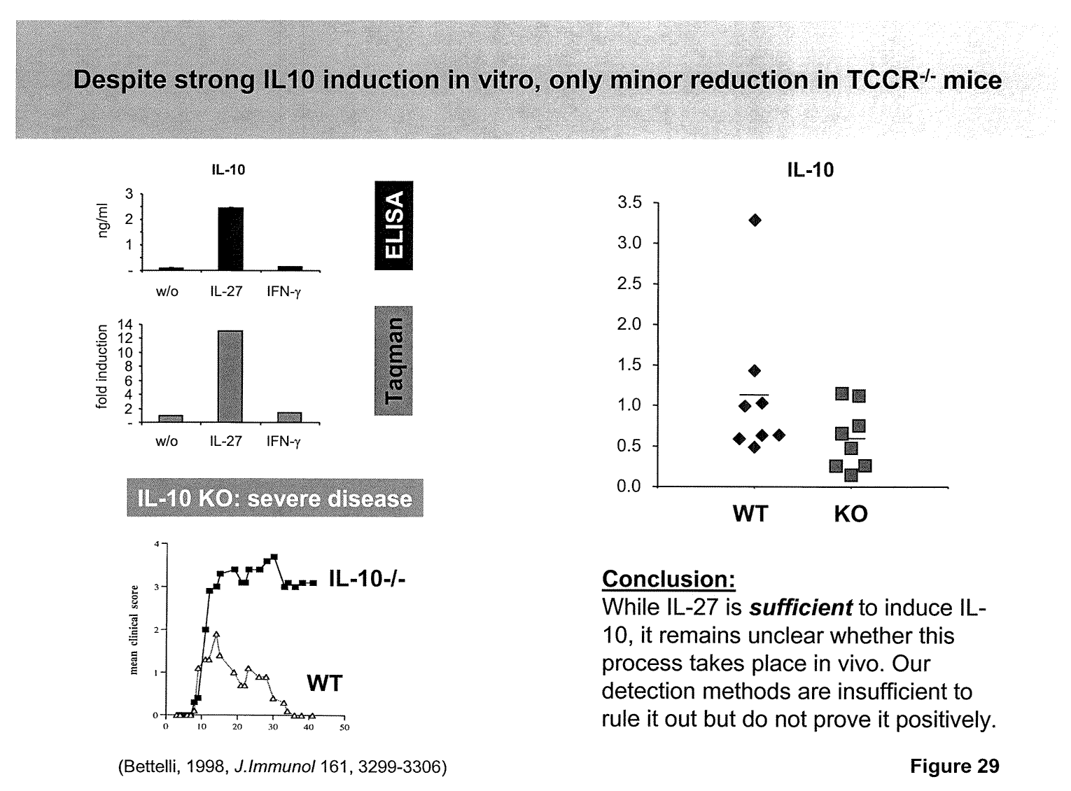

To determine whether any of these effects were secondary to induction of the well-known and potent immunosuppressive cytokine IL-10, the effects of IL-27 were also examined in IL-10 deficient T-cells. As shown in FIG. 29, IL-27 induced modulation of cytokine production was independent of IL-10, as little difference was seen in IL-2 or GM-CSF production comparing WT and IL-10 deficient T-cells.

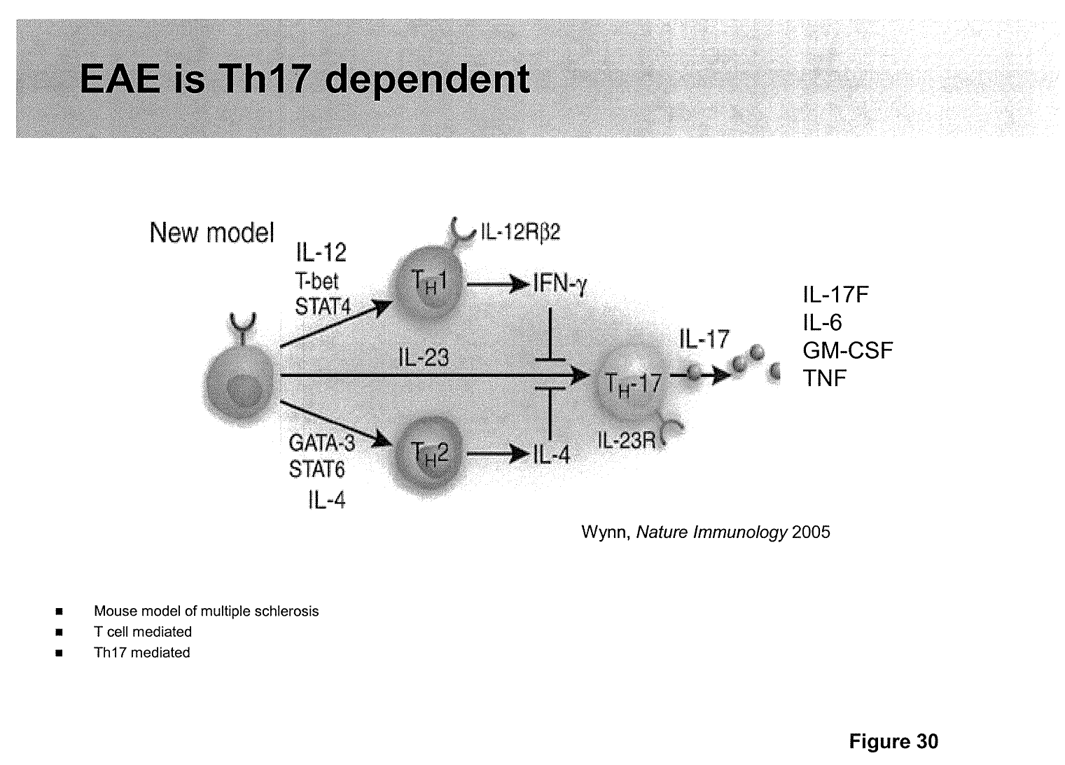

Despite strong induction of the immunosuppressive IL-10 by IL-27 seen in vitro (FIG. 28), only a minor reduction of IL-10 was seen in T-cells from IL-27R −/− mice with EAE (FIG. 30). However, this artificial in vitro observation does in no way preclude the interpretation that IL-27 mediated IL-10 induction is an important biological process during EAE. On the contrary, it most likely reflects the limits of the experimental techniques at our disposal to study IL-27 induced IL-10 induction in vivo.

### Example 11

### EAE is TH-17 Dependent

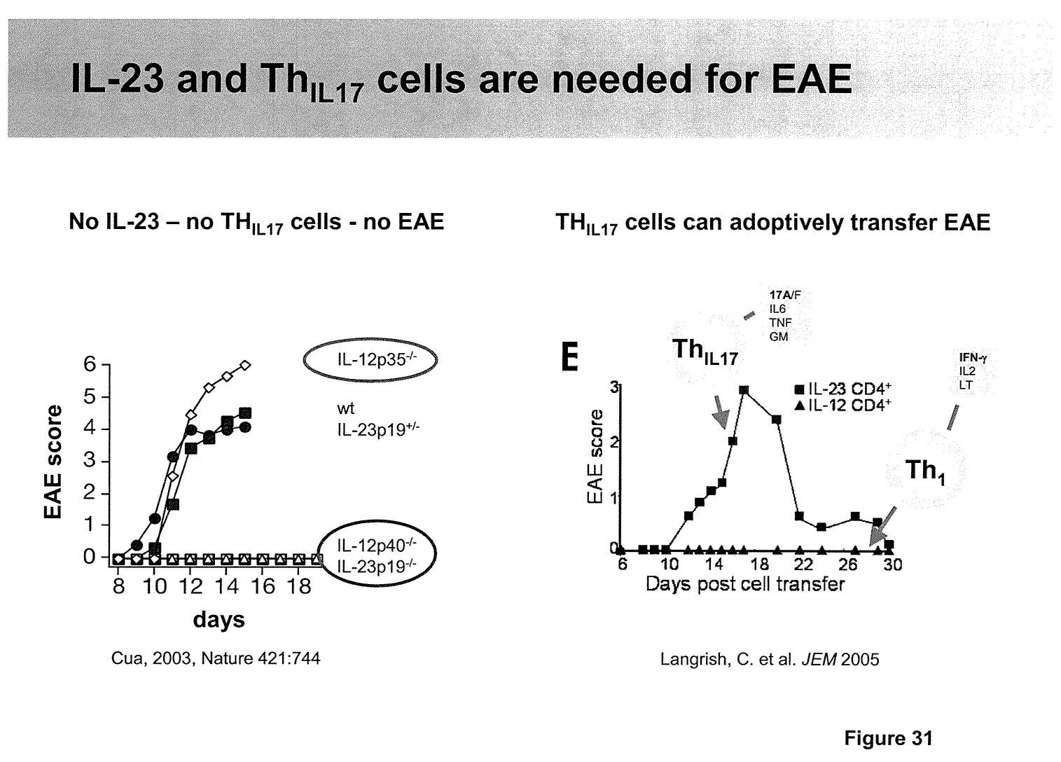

Recent evidence suggests that a new subtype of helper T-cells, so called TH-17 cells, are key mediators of many pro-inflammatory processes, including EAE. These Th-17 cells were reported to produce IL-17A, IL-17F, IL-6, TNF, and GM-CSF. See the diagram provided in FIG. 31. The development of TH-17 cells is poorly understood, but is thought to be dependent on IL-23, another heterodimeric cytokine with similarity to IL-12. IL-23 deficient animals cannot develop this T-cell phenotype efficiently and are resistant to EAE and CIA. However, while IL-23 appears to be necessary, it is not sufficient for TH-17 cell differentiation in vitro.

As discussed above, IL-27R deficient mice developed more severe EAE disease as compared to WT littermates. Events downstream to IL-27 signaling were analyzed to determine factor important in this limiting effect on the severity of EAE. The expression of a variety of cytokines in response to IL-27 was examined during activation.

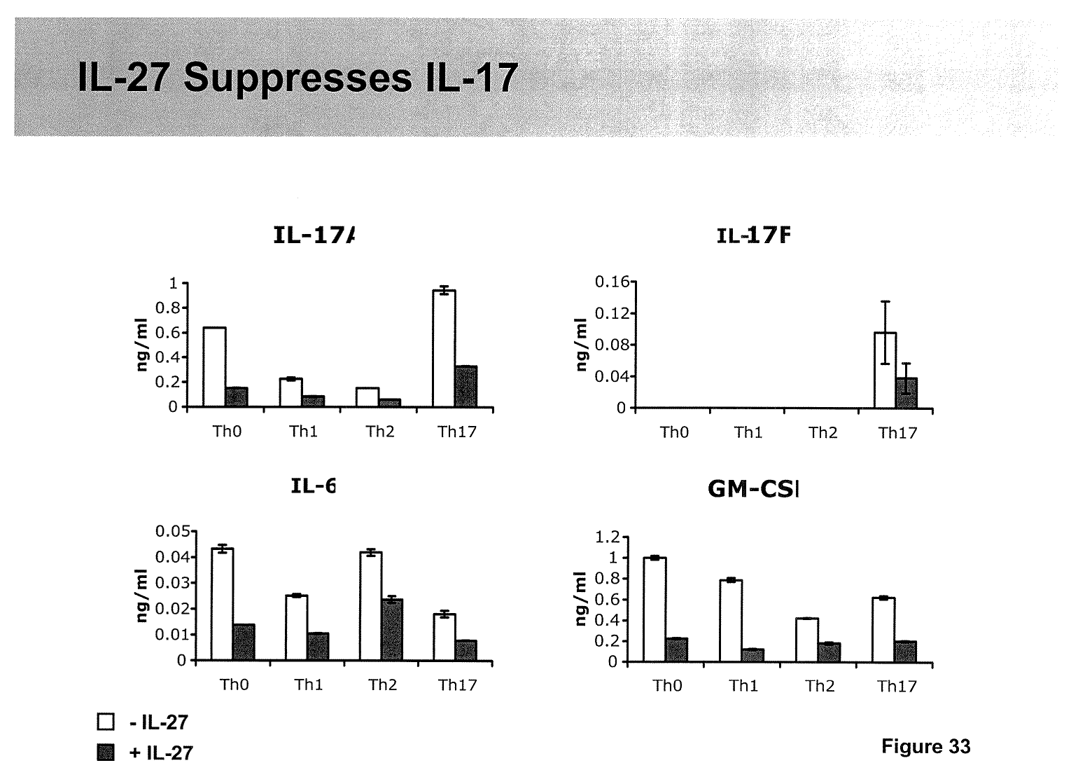

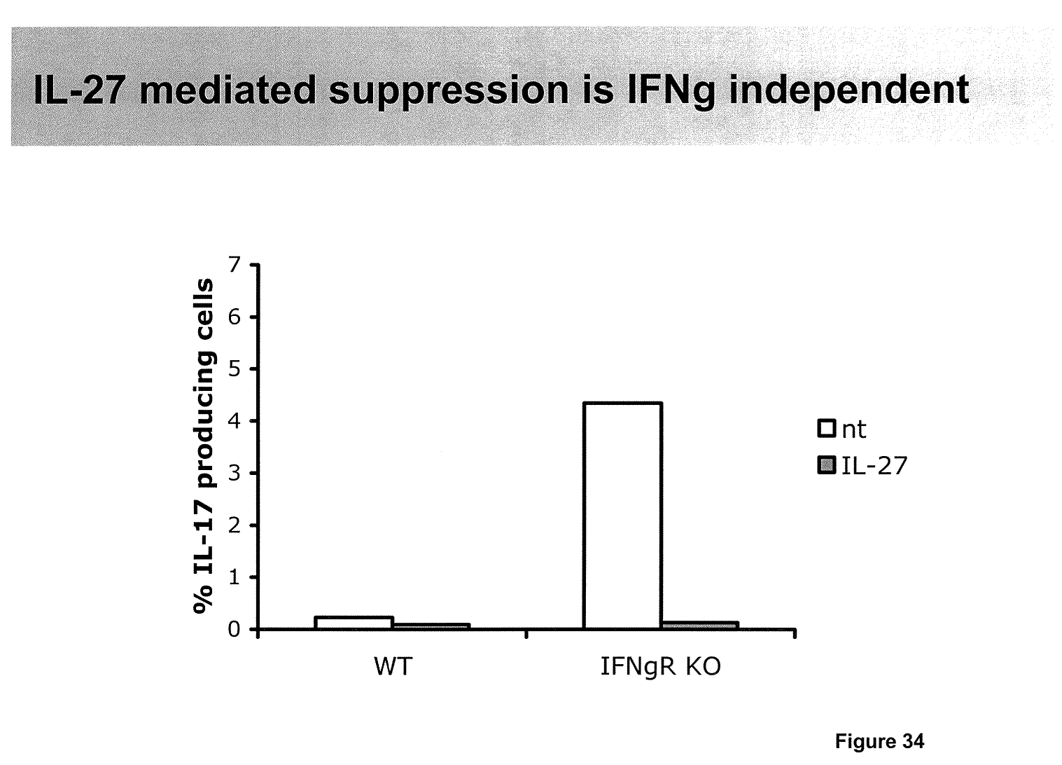

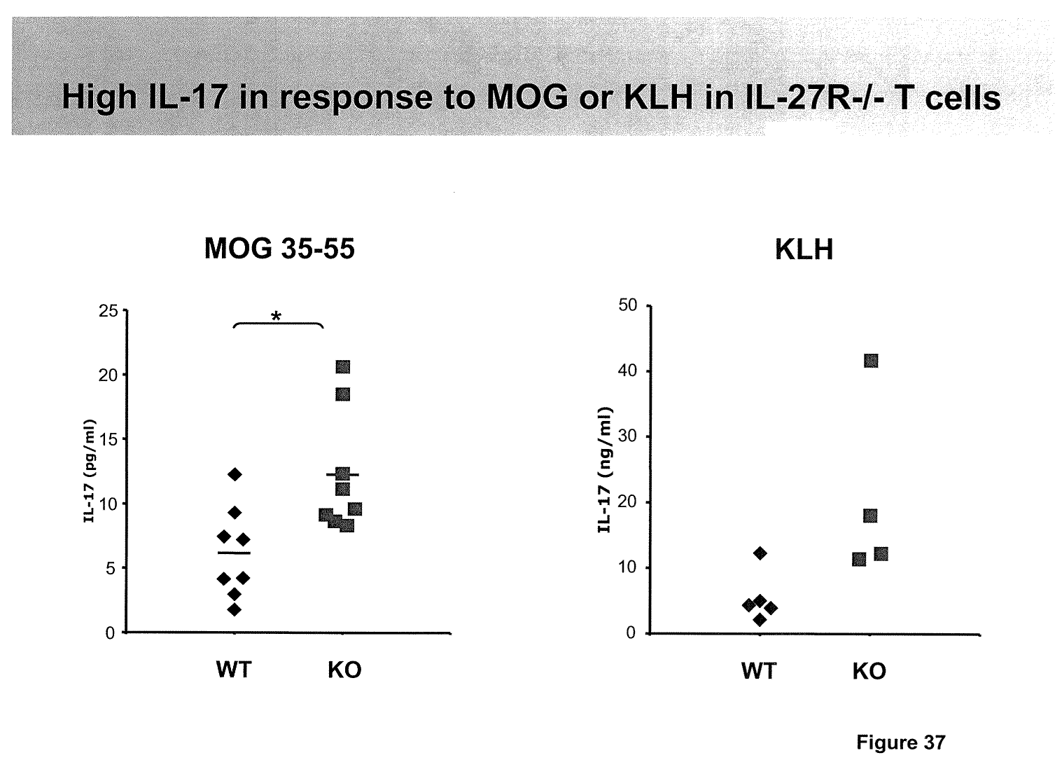

IL-27 promotes IFN-gamma production, and IFN-gamma is known to inhibit IL-17. The data demonstrates that IL-27 suppressed production of IL-17 and other Th-17 cytokines IL-6 and GM-CSF more efficiently than did IFN-gamma (See FIGS. 33 and 34). Furthermore, lymph node cells from TCCR−/− mice with EAE secreted more Th-17 cytokines upon re-stimulation in vitro than WT (FIG. 37).

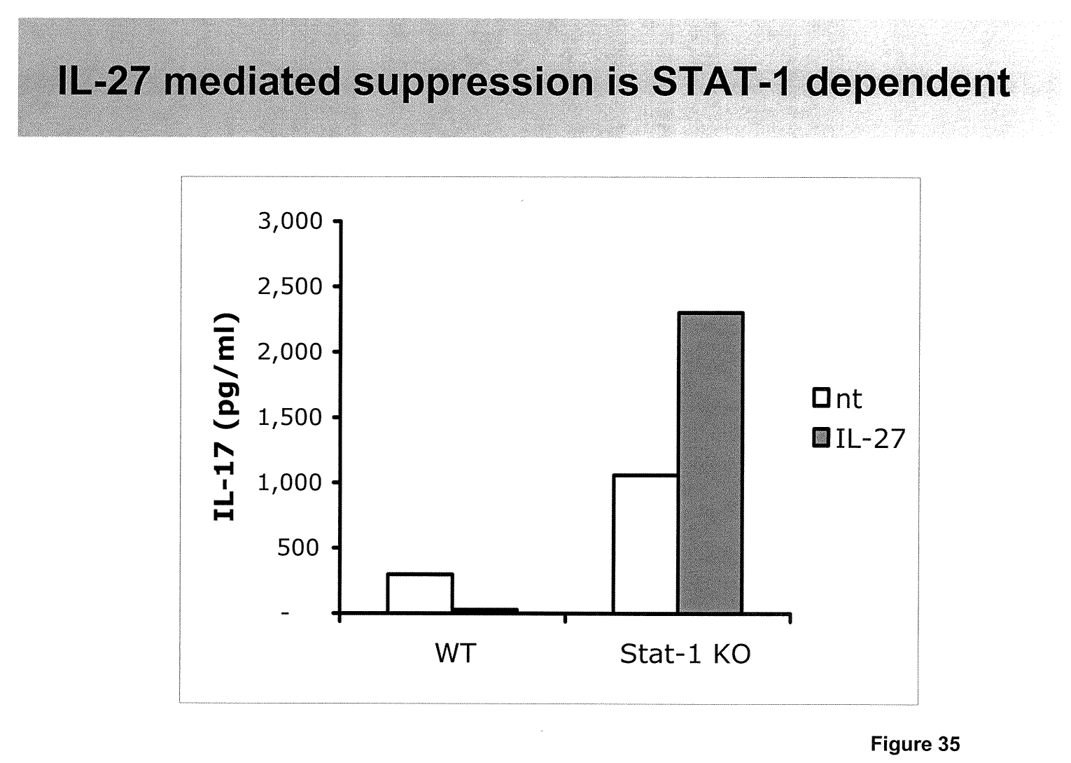

The IL-27 mediated suppression of IL-17 production was independent of IFN-gamma, because T-cells rendered non-responsive to IFN-gamma still suppressed IL-17 production upon stimulation with IL-27. (FIG. 35). In the absence of IFN-gamma signaling, the basal IL-17 production was higher. The reason for this is unclear, because even in WT cultures, IFN-gamma signaling is blocked by addition of IFN-gamma neutralizing antibodies. Thus, the high IL-17 expression in IFN-gammaR deficient mice could either reflect a developmental alteration (i.e. IFNgR deficient T-cells are different from WT-cells in more than the expression of IFNgR), or, alternatively, could reflect an intracellular IFNg loop. In cells where a ligand and a receptor are co-expressed, signaling can occur within the late secretory pathway, and such signaling would be intracellular and not blocked by neutralizing antibodies.

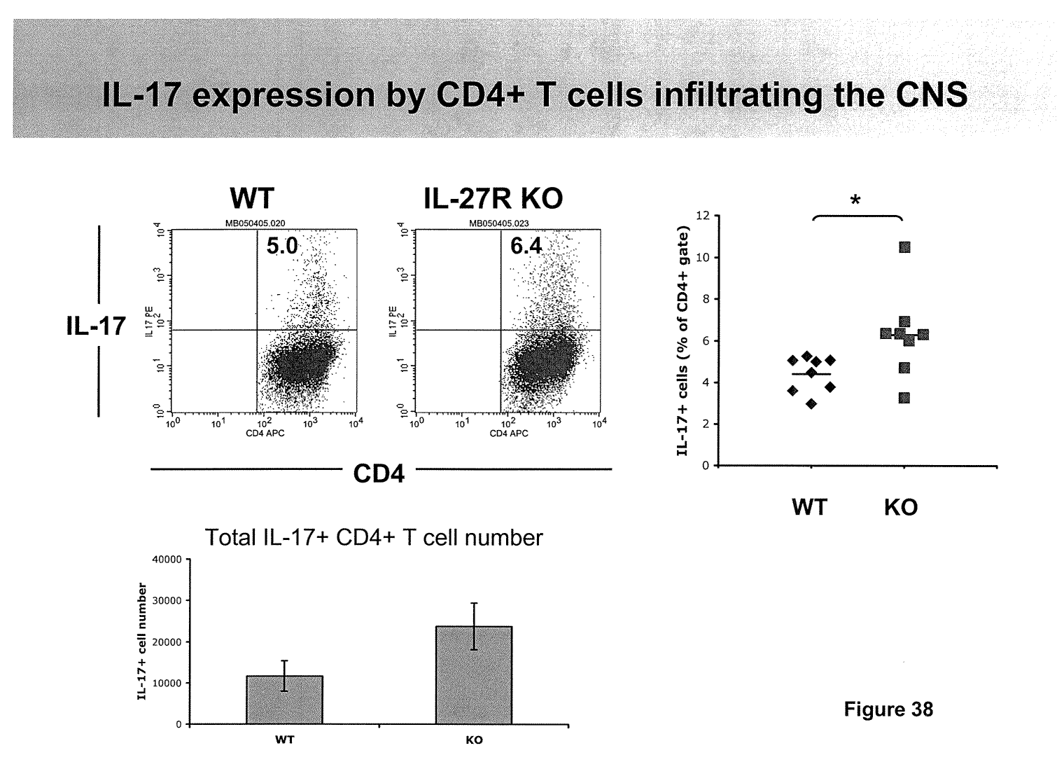

To determine if Th-17 cells were dysregulated in IL-27R deficient mice, IL-27R deficient mice were immunized with MOG in CFA. Draining lymph nodes were removed at 14 days and re-stimulated with MOG ex vivo. Lymph node supernatants containing IL-27R deficient T cells expressed significantly increased levels of IL-17 (FIGS. 37 and 38).

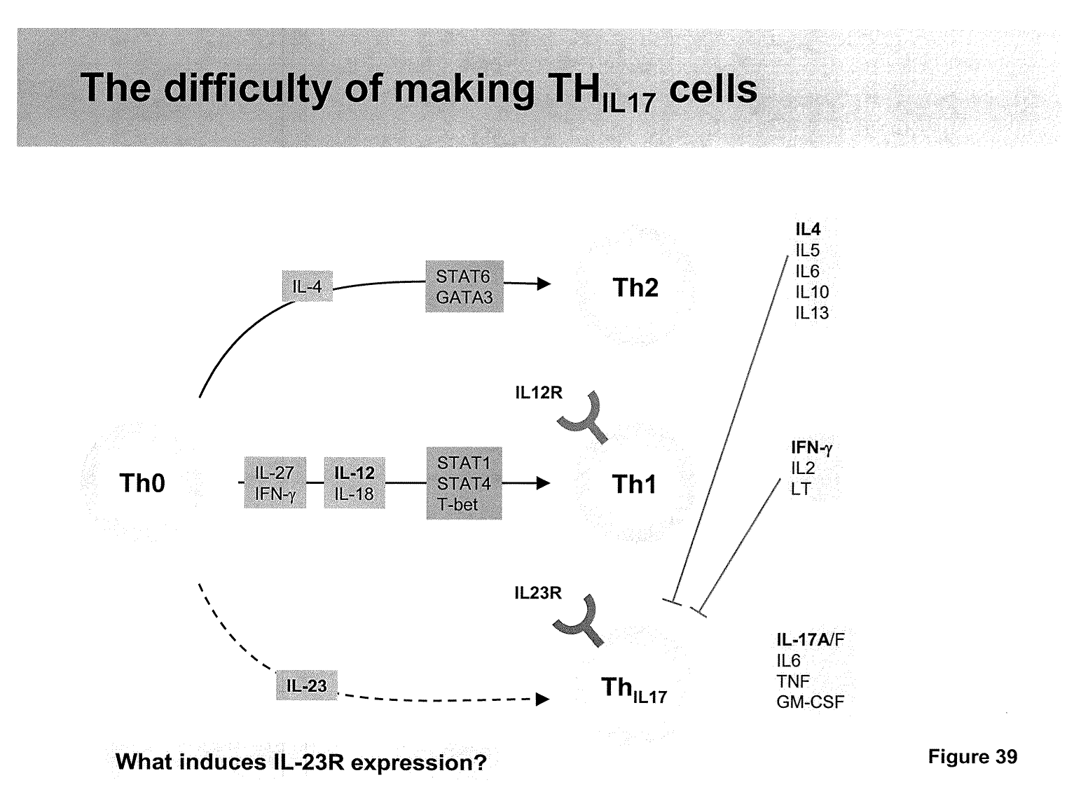

Furthermore, analysis of the immune infiltrate of brain and spinal cord (the actual site of inflammation in EAE) revealed that more cells infiltrated in IL-27R deficient mice. Furthermore, a higher percentage of these cells were IL-17 positive when analyzed by intracellular staining. Together, these two observations translate into roughly two-fold expression of IL-17 in the spinal cord. (See FIG. 39).

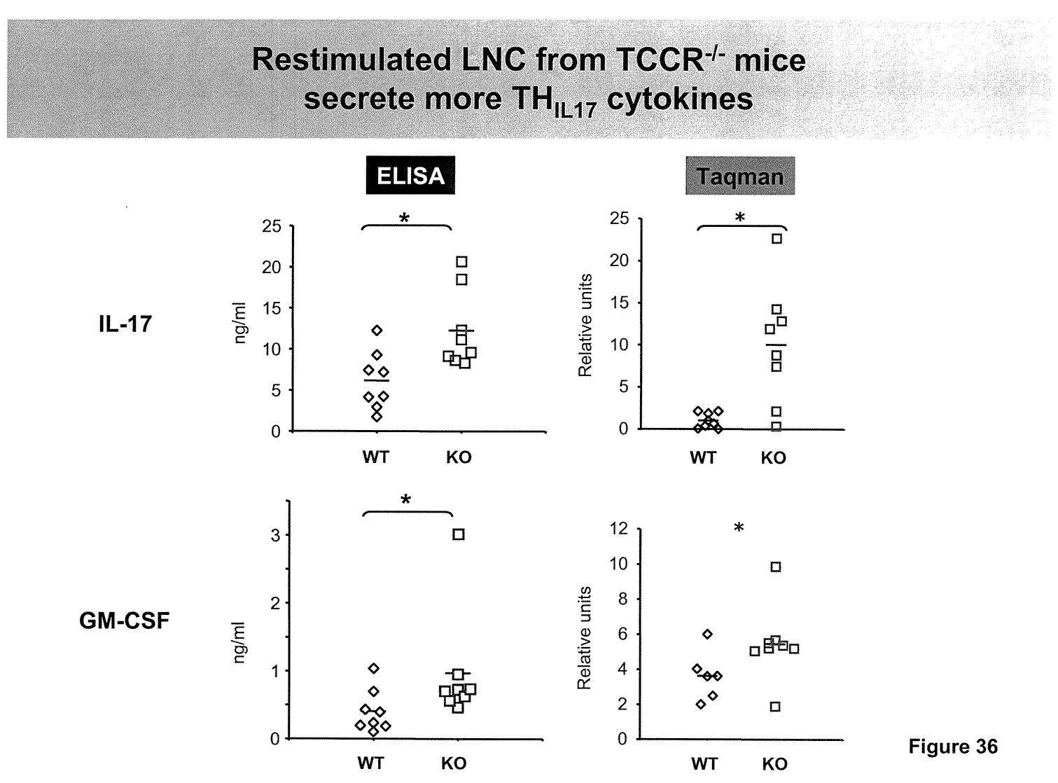

Both IFN-gamma and IL-27 activate STAT-1 and STAT-1 knockouts produce increased IL-17. Accordingly, IL-27 may suppress IL-17 by activating STAT-1. This relationship was investigated by analyzing IL-27 mediated suppression of IL-17 in cells obtained from a STAT-1 knockout model. In the absence of STAT-1, IL-27 did not suppress IL-17, indicating that the suppression is mediated by STAT-1. In the absence of STAT-1, IL-27 becomes an inducer of IL-17. The mechanistic basis for this reversal is unknown, but it is fair to speculate that activation of STAT-3 by IL-27 plays a role in this effect, because other IL-17 inducing cytokines (notably IL-23) signal through STAT-3 while not activating STAT-1 (See FIG. 36).

In summary, IL-27 receptor (TCCR) deficient mice are EAE-hypersensitive. IL-27 effectively suppressed Th-17 cytokines IL-17, IL-6, and GM-CSF in vitro. Furthermore, IL-27 receptor deficient mice with EAE produce more Th-17 cytokines than wild type. IL-27 may suppress EAE by skewing the immune response away from Th-17.

### Example 12

### IL-6 Induces Th-17 Cells

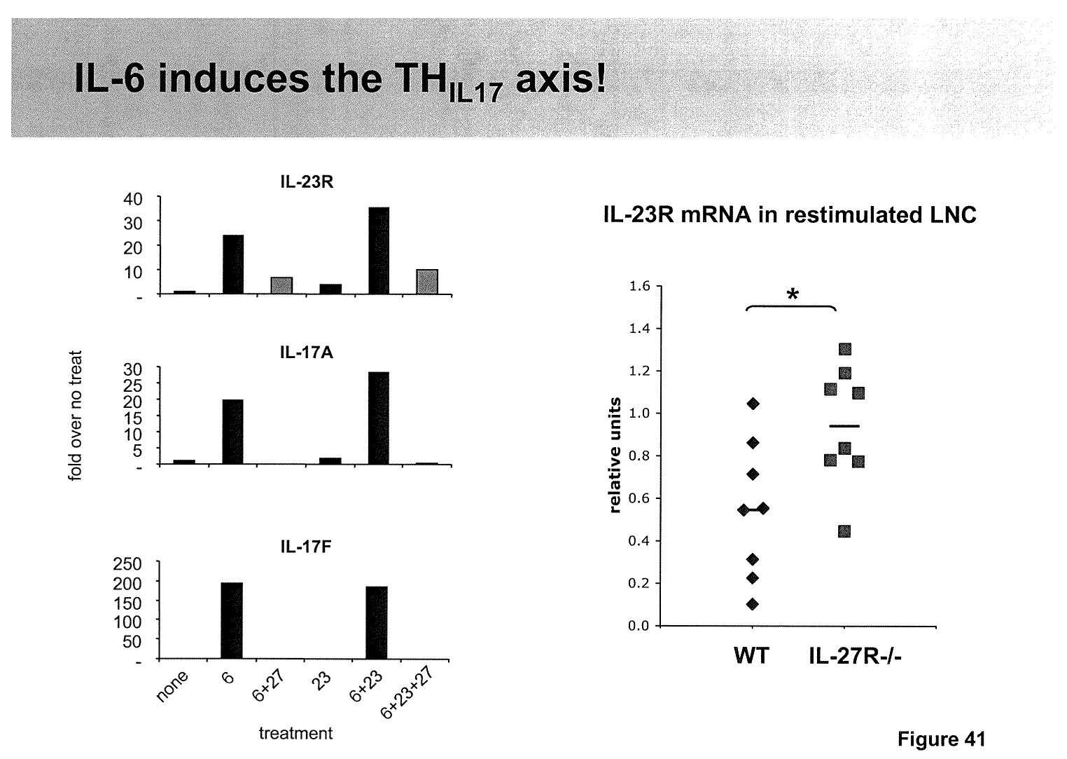

As shown diagrammatically in FIG. 41, IL-23 is necessary but not sufficient for the differentiation of Th-0 cells into Th-17 cells that produce cytokines IL-17, IL-6, GM-CSF, and TNF. One likely reason why IL-23 is not sufficient is that Th-0 cells do not express the IL-23 receptor and are therefore IL-23 non-responsive. Therefore, a factor capable of inducing IL-23R in Th-0 cells is a mandatory component of the TH-17 differentiation pathway.

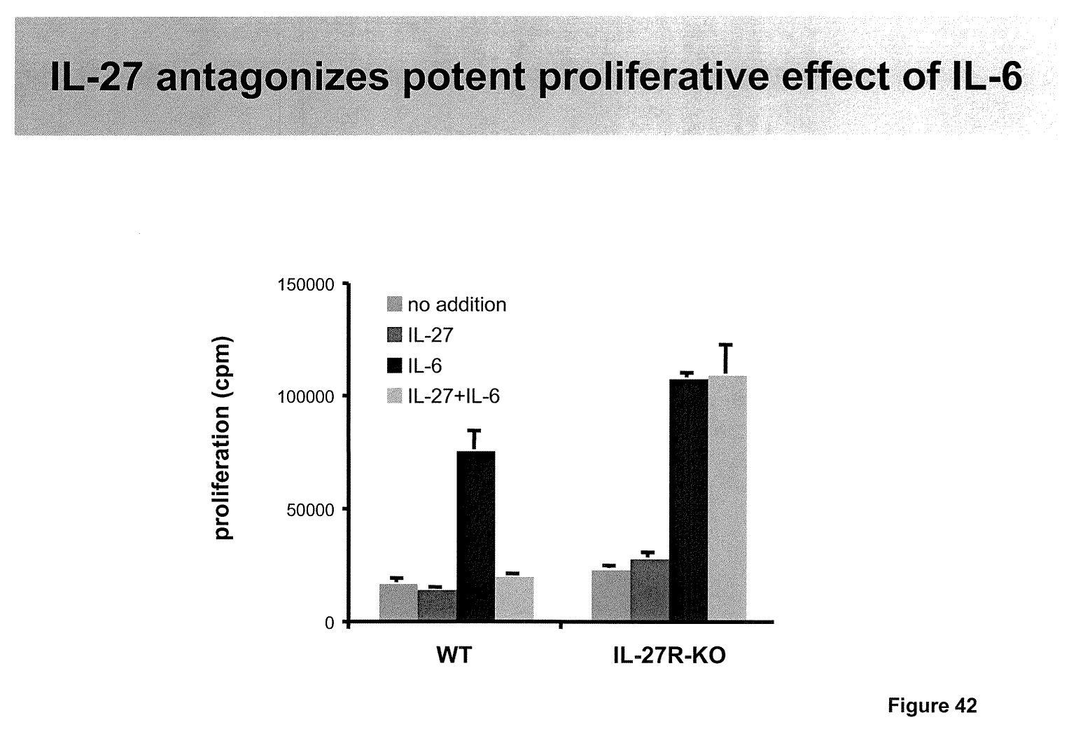

Since effector cytokines of TH-1 (IFN-g) and TH-2 (IL-4) cells also participate in the development of these cells and hence provide a stabilizing feedback loop, we reasoned that one of the TH-17 effector cytokines must, by analogy, participate in TH-17 development. Among the TH-17 effector cytokines, IL-6 looks most promising, because its receptor is expressed on naive T-cells, and because there are other sources (most notably antigen presenting cells) of IL-6 than terminally differentiated T-cells. In addition, IL-6 knockout mice are EAE resistant (See FIG. 42).

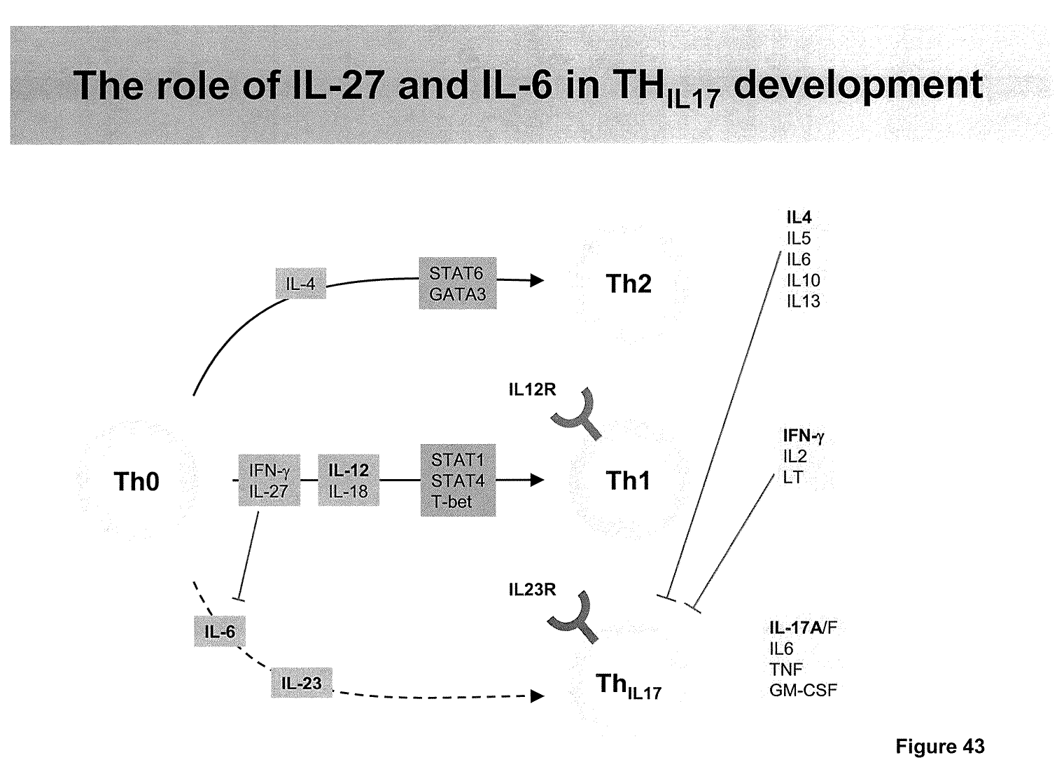

Wild type and IL-27 receptor knockout mice were examined for response to IL-6 alone, or in combination with IL-27 and IL-23. As shown in FIG. 43, IL-6 induced the Th-17 axis. Treatment with IL-6 alone stimulated IL-23 receptor and also stimulated IL-17A and IL-17F production. Interestingly, co-administered IL-27 reduced or eliminated the IL-6 stimulated increase in IL-23 receptor and IL-17 production (See FIG. 43). IL-23 had a slight effect on the stimulation of IL-23 receptor and IL-17 production that appeared to be additive to the large stimulation demonstrated for IL-6 alone. The addition of IL-27 to this combination also reduced or eliminated the response. mRNA taken from re-stimulated lymph node cells showed induction of IL-23 Receptor in the IL-23 receptor knockout as compared with wild type control (See FIG. 43).

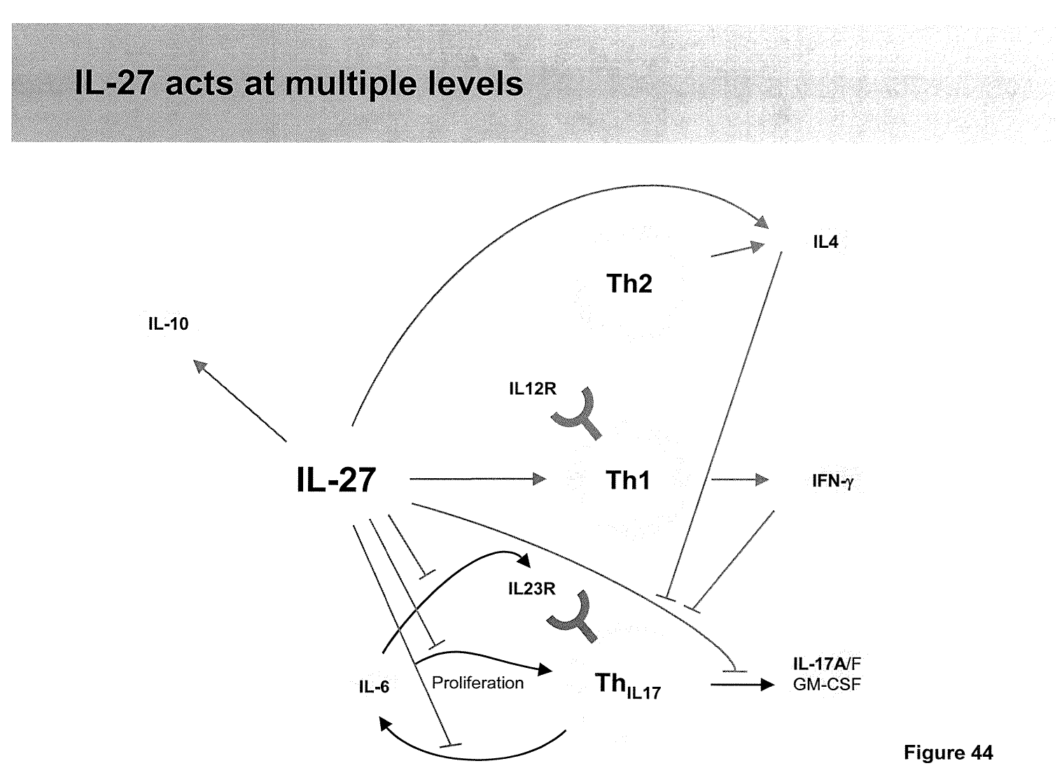

Further comparing the effects of IL-6 in a proliferation assay, IL-6 stimulated greatly enhanced proliferation of purified T-cells in both wild type and TCCR knockout mice. The addition of IL-27 completely neutralized IL-6 induced proliferation in wild-type cells. This reduction was not seen, however, in the TCCR knockout mice, demonstrating that IL-27 antagonizes potent proliferative effects of IL-6. See FIG. 44. Therefore, it appears that IL-27 is an IL-6 antagonist on several levels, including IL-6 driven TH-17 differentiation.

### Example 13

### Role of IL-27

As shown in FIG. 46, IL-27 impacts differentiation of T cells, particularly the development of Th-17 cells at multiple levels. While IL-27 stimulates production of IL-10, IL-4, and development of Th-1 cells, it also suppresses production of Th-17 cells, production of Th-17 cell cytokines IL-17 and GM-CSF.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many

1. A method of treating an autoimmune disorder comprising administering a TCCR agonist.

2. (canceled)

3. A method of increasing IL-10 expression in lymphocytes comprising administering to the lymphocytes a TCCR agonist.

4. A method of increasing SOCS3 expression in lymphocytes comprising administering to the lymphocytes a TCCR agonist.

5. The method of claim 1, wherein the TCCR agonist is an antibody, or an active fragment thereof.

6. The method of claim 5, wherein the TCCR agonist antibody is a monoclonal antibody.

7. The method of claim 6, wherein the monoclonal antibody is produced by the hybridoma cell line deposited under American Type Culture Collection Accession Number ATCC PTA-6447.

8. The method of claim 5, wherein the TCCR agonist antibody is a humanized antibody.

9. The method of claim 1, wherein the TCCR agonist is a TCCR variant that can dampen or suppress a Th1 response.

10. The method of claim 1, wherein the TCCR agonist is an antibody fragment or a single-chain antibody.

11. The method of claim 1, wherein the agonist comprises a TCCR extracellular domain.

12. The method of claim 1, wherein the agonist comprises IL-27 or a portion thereof.

13. The method of claim 1, wherein the agonist comprises an IL-27 variant.

14. The method of claim 1, wherein the agonist comprises an IL-27 variant comprising a portion of p28 capable of binding TCCR and gp130.

15. The method of claim 1, wherein the agonist is a fusion protein comprising a portion of IL-27 that can dampen or suppress a Th1 response and a heterologous peptide.

16. The method or use of claim 15, wherein the heterologous peptide comprises an Fc portion of an antibody.

17. The method of claim 1, wherein the autoimmune disorder is allograft rejection.

18. The method of claim 1, wherein the autoimmune disorder is an autoimmune thyroid disease.

19. The method of claim 1, wherein the autoimmune disorder is autoimmune uveoretinitis.

20. The method of claim 1, wherein the autoimmune disorder is giant cell arteritis.

21. The method of claim 1, wherein the autoimmune disorder is an inflammatory bowel disease.

22. The method of claim 1, wherein the autoimmune disorder is insulin-dependent diabetes mellitus.

23. The method of claim 1, wherein the autoimmune disorder is multiple sclerosis.

24. The method of claim 1, wherein the autoimmune disorder is pernicious anemia.

25. The method of claim 1, wherein the autoimmune disorder is psoriasis.

26. The method of claim 1, wherein the autoimmune disorder is rheumatoid arthritis.

27. The method of claim 1, wherein the autoimmune disorder is sarcoidosis.

28. The method of claim 1, wherein the autoimmune disorder is scleroderma.

29. The method of claim 1, wherein the autoimmune disorder is systemic lupus erythematosus.

30. The method of claim 1, wherein the autoimmune disorder is at least partially mediated by a Th1 response.

31. The method of claim 1, wherein the autoimmune disorder is at least partially mediated by CD8⁺ T-cell proliferation.

32. A method of screening for TCCR agonists, comprising:
contacting a cell expressing TCCR with a candidate TCCR agonist;
analyzing expression of a TCCR-activated gene in response to the candidate TCCR agonist; and
correlating an increase in expression of the TCCR-activated gene with activity of the candidate TCCR agonist.

33. The method of claim 32, wherein the TCCR-activated gene encodes IL-10, SOCS-3, or both.

34. The method of claim 32, wherein the TCCR-activated gene encodes SOCS-3.

35. The method of claim 32, wherein said cells are T-lymphocytes.

36. The method of claim 32, wherein said analyzing comprises quantitative PCR analysis.

37. The method of claim 32, wherein said analyzing comprises immunoassay analysis.

38. A monoclonal antibody produced by a hybridoma cell line deposited under American Type Culture Collection Accession Number ATCC PTA-6447.

39. A hybridoma cell line deposited under American Type Culture Collection Accession Number ATCC PTA-6447.

40. A method for treating or suppressing an immune response comprising administering IL-27 or an agonist thereof.

41. The method of claim 1, wherein the immune response is mediated by Th-17 cells.

42. The method of claim 1, wherein the immune response is a Th-1 or Th2 mediated response.

43. The method of claim 1, wherein the immune response is a hyperinflammatory response.

44. The method of claim 1, wherein the immune response is an autoimmune response.

45. The method of claim 1, wherein the IL-27 suppresses IL-17 production.

46. A method for inhibiting IL-17, IL-6, or GM-CSF production comprising administering IL-27 or an agonist thereof.

47. A method for treating or suppressing an immune response comprising administering an an antagonist of IL-6 or its receptor.

48. The method of claim 47, wherein said antagonist is an antibody, aptomer, or small molecule antagonist that blocks activation of IL-6 with its receptor.


SEQUENCE LISTING (20 sequences)

SEQ ID NO: 1 - protein, 636 aa, Human TCCR [sequence omitted]

SEQ ID NO: 2 - protein, 623 aa, Murine TCCR
MNRLRVARLTPLELLLSLMSLLLGTRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHSGSFKLWVTVSTVAGQGPPGPDLSLHLPDNRIRWKALPWFLSLWGLLLMGCGLSLASTRCLQARCLHWRHKLLPQWIWERVPDPANSNSGQPYIKEVSLPQPPKDGPILEVEEVELQPVVESPKASAPIYSGYEKHFLPTPEELGLLV

SEQ ID NO: 3 - protein, 243 aa, Human p28
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 4 - protein, 234 aa, Murine p28
MGQVTGDLGWRLSLLLLPLLLVQAGSWGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 5 - protein, 229 aa, Human EBI3
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 6 - protein, 918 aa, Human gp130 [sequence omitted]

SEQ ID NO: 7 - protein, 21 aa, MOG 35-55
MEVGWYRSPFSRVVHLYRNGK

SEQ ID NO: 8 - protein, 11 aa, Ac - 1-11
ASQKRPSQRHG

SEQ ID NO: 9 - protein, 21 aa, mSOCS1 forward
TGGTTGTAGCAGCTTGTGTCT

SEQ ID NO: 10 - protein, 25 aa, mSOCS1 reverse
GTGCAAAGATACTGGGAATATGTAA

SEQ ID NO: 11 - protein, 27 aa, mSOCS1 probe
CCAGGACCTGAATTCCACTCCTACCTC

SEQ ID NO: 12 - protein, 22 aa, mSOCS3 forward
TCCTGAGTTAACACTGGGAAGA

SEQ ID NO: 13 - protein, 19 aa, mSOCS3 reverse
GGAGGCTCTCGGACCTACT

SEQ ID NO: 14 - protein, 27 aa, mSOCS3 probe
ATTGGCCAGTCCTAGTCATCTCTCGGT

SEQ ID NO: 15 - protein, 20 aa, mPIAS1 forward
GATGGCAACTGATGGAGGAT

SEQ ID NO: 16 - protein, 19 aa, mPIAS1 reverse
AGTGCAGGAGCTGGTGATG

SEQ ID NO: 17 - protein, 24 aa, mPIAS1 probe
TGTGCCCTGGCTCTCTGCAGTTAC

SEQ ID NO: 18 - protein, 18 aa, MPIAS3 forward
ATCCCTCAGGGGTCATTG

SEQ ID NO: 19 - protein, 18 aa, MPIAS3 reverse
GGCCAAAAGCAGGTATCC

SEQ ID NO: 20 - protein, 22 aa, mPIAS3 probe
CAAAGGCCAGGCCAGAGCTTCA